(19) United States
(12) Patent Application Publication
(10) Pub. No.: US 2023/0372473 A1
(43) Pub. Date: Nov. 23, 2023

(54) VACCINE COMPOSITIONS

(71) Applicant: VIROKINE THERAPEUTICS LIMITED, London (GB)

(72) Inventor: Ursula Adele Gompels, London (GB)

(73) Assignee: Virothera Limited, Cambridge (GB)

(21) Appl. No.: 18/030,633

(22) PCT Filed: Sep. 24, 2021
(86) PCT No.: PCT/GB2021/052485

§ 371 Date: Apr. 6, 2023

(30) Foreign Application Priority Data
Oct. 8, 2020 (GB) 2015984.4
May 19, 2021 (GB) 2107170.9

(51) Int. Cl. A61K 39/245; A61P 37/04

(52) U.S. Cl. CPC A61K 39/245; A61P 37/04; A61K 2039/53

(57) ABSTRACT

A sterile pharmaceutical composition comprising one or more nucleic acid molecules comprising a plurality of immunogen coding regions which collectively encode a plurality of herpesvirus polypeptides, wherein the one or more nucleic acid molecules are capable of expressing the plurality of herpesvirus polypeptides when introduced into a vertebrate cell in an expression vector, wherein each of the plurality of immunogen coding regions has at least 90% sequence identity to a native coding region for a corresponding native full-length herpesvirus polypeptide from the same herpesvirus species, wherein the plurality of herpesvirus polypeptides are: (i) gD of herpes simplex virus 2 or herpes simplex virus 1; gE or gl of varicella zoster virus; gp350 or gp42 of Epstein Barr virus; gp42 of Epstein Ban virus, gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and (ii) gB, gH and gl of the respective cognate human herpesvirus; and wherein the pharmaceutical composition is provided in a sealed sterile container for delivery.

### FIELD OF THE INVENTION

The invention relates to vaccine or immune advanced-therapies compositions, particularly polynucleotide vaccines, suitable for use against infectious agents, particularly herpesvirus.

### BACKGROUND OF THE INVENTION

Human herpesviruses are a group of membrane enveloped double stranded DNA viruses responsible for significant global morbidity and mortality in humans (Knipe and Howley, 2013) All these viruses use membrane fusion to initiate cellular infection (Eisenberg et al., 2012; Vollmer and Grunewald, 2020). There are nine known human herpesvirus species classified (Davison et al., 2009) (ICTVonline.org) as HHV1, HHV2, HHV3, HHV4, HHV5, HHV6A, HHV6B, HHV7 and HHV8. These also include ICTV classification and common names (i) HHV1, human alphaherpesvirus 1, has the common name herpes simplex virus type 1 (HSV1); (ii) HHV2, human alphaherpesvirus 2, herpes simplex virus type 2 (HSV2); (iii) HHV3, human alphaherpesvirus 3, varicella-Zoster virus (VZV); (iv) HHV4, human gammaherpesvirus 4, Epstein Barr virus (EBV); (v) HHV5, human betaherpesvirus 5, human cytomegalovirus (HCMV); (vi) HHV6A, human betaherpesvirus 6A (vii) HHV-6B, human betaherpesvirus 6B and (viii) HHV7, human betaherpesvirus 7; and (viii) HHV8 is Kaposi's sarcoma-associated herpesvirus (KSHV). In humans, these viruses cause a wide variety of disease, with those most severe as follows. HSV1 causes oral herpes and encephalitis, HSV2 causes genital herpes, and neonatal herpes with high fatality and lifelong disability, and VZV causes chickenpox and shingles. EBV causes infectious mononucleosis and is strongly associated with several B cell lymphomas, nasopharyngeal carcinoma, and gastric adenocarcinoma. HCMV causes severe infection in immunosuppressed patients and one of the most prevalent congenital infections resulting in the leading non-genetic cause of hearing loss as well as lifelong disability. HHV6A, HHV6B and 7 cause roseola infantum (Sixth disease), post transplant limbic encephalitis and associated with neurological disease, and HHV-8 causes Kaposi's sarcoma in several clinical settings including in patients infected with human immunodeficiency virus (HIV).

The commonly used representative model of the human herpesvirus and the type species for the ‘alpha’ herpesvirus (alphaherpesvirinae) subfamily are HSV. Despite intensive research, there are no effective vaccines for HSV1 or HSV2 and apart from vaccines for VZV, none are available for any of the other human herpesvirus. Nevertheless, much has been learnt regarding HSV immune responses. Over 20 years of research has firmly established that the envelope glycoprotein D from herpes simplex virus is a major immunogen. It is responsible for the virus binding to the cell by attaching to cellular receptors including within the TNF-R and Ig family, herpes virus entry mediator (HVEM), nectin or 3-O-sulfated-heparan sulfate (3-OS-HS) (Hilterbrand and Heldwein, 2019). The role of gD as a major immunogen has been clearly demonstrated using subunit proteins prepared using recombinant antigens, and also using DNA, which encodes the glycoprotein. In preclinical trials using both murine and guinea pig models of infection, immunisations with either gD subunit protein or the encoding DNA have provided some protection from acute challenge infection. Both also generate neutralising antibody. Moreover, clinical trials using the HSV2 gD subunit in a vaccine formulation with chemical adjuvants alum and MPL showed effective generation of neutralising antibodies together with protection for women from HSV1, although not from HSV2 (Belshe et al., 2012). The immune correlate of protection for the trial was shown to be levels of neutralising antibodies (Belshe et al., 2014). It appeared a further boost was required to make a sufficiently protective HSV2 vaccine and it was posited that other immune evasion glycoproteins could affect the response. Analyses of the guinea pig model of infection showed that this vaccine would require boosting for protection (Bernstein, 2020; Stanberry et al., 2002). Nonetheless it has been a useful comparator for evaluating new modalities for vaccination or immunotherapy for HSV1 and HSV2 infections.

The glycoprotein gD has been combined with other herpesvirus proteins in various proposed subunit protein vaccine formulations; and other formulations using genetically engineered virus with gene deletions lacking gD have also been proposed. U.S. Pat. No. 9,555,099 B2 discloses vaccine compositions comprising recombinant HSV2 proteins and an adjuvant; the protein component including an envelope glycoprotein such as gD, and structural proteins other than an envelope glycoprotein e.g. capsid or tegument protein. U.S. Pat. No. 7,094,767 B2 discloses DNA vaccines for HSV2 expressing full-length HSV2 gD and/or truncated gB, another herpesvirus envelope protein. US2019/0367561 A1 principally relates to vaccine compositions for EBV or HCMV, and discloses antigenic compositions comprising at least two human herpesvirus (HHV) polypeptides involved in mediating HHV binding, fusion and entry into host cells, such as gp350 extracellular domain, gH extracellular domain, gL and gB extracellular domain, or encoding nucleic acids. These may be combined with other polypeptides, such as gD, implying the possibility of combining nucleic acid molecules and polypeptides in the same composition. The approach of combining nucleic acid molecules and polypeptides in prime boost vaccination approaches is known in the art (Muthumani et al., 2013). Typical studies have focused on truncated versions of polypeptides in order to avoid ER retention for example in the case of gB or to have secreted forms of gD to increase exogenous antigen exposure. Despite the manifold possible combinations of herpesvirus polypeptides that may be included in a vaccine, many mainly using molecules exposed on the exterior of the virion or expanding available epitopes without further rationale, there is a lack of evidence that combinations of polypeptides are more effective in vivo at protecting against virus challenge than immunisation with gD alone. In a guinea pig model, polypeptide vaccines comprising combinations of truncated gD; truncated gD plus gB and gH/gL; or gB and gH/gL raised effective immunity, but no vaccine was significantly more effective than the vaccine comprising truncated gD as sole polypeptide, and T cell boosting was still required (Bernstein et al., 2011).

Polynucleotide vaccines may be simpler, safer and more economic to produce and store than polypeptide vaccines, can stimulate cellular immunity since antigen is produced intracellularly and may be preferred for these reasons. However, they generally require an adjuvant boost (Liu, 2019). Vaccine effects may be modulated by different adjuvants, or vectors used in delivery of a polynucleotide. To date much focus of polynucleotide vaccine research has been increasing the antigenic dosage via increasing nucleic acid delivery or by using chemical or genetic adjuvants (Gary and Weiner, 2020; Grunwald and Ulbert, 2015; Liu, 2019).

There remains a need for effective herpesvirus vaccines, particularly rationally designed vaccines, which are designed based on a clear scientific rationale.

The listing or discussion of a prior-published document in this specification should not be taken as an acknowledgement that the document is part of the state of the art or is common general knowledge.

### SUMMARY OF THE INVENTION

A first aspect of the invention provides a pharmaceutical composition comprising one or more nucleic acid molecules comprising a plurality of immunogen coding regions which collectively encode a plurality of herpesvirus polypeptides, wherein the one or more nucleic acid molecules are capable of expressing the plurality of herpesvirus polypeptides when introduced into a vertebrate cell, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus; and

wherein the pharmaceutical composition is sterile, and is provided in a sealed sterile container.

A second aspect of the invention provides a pharmaceutical composition comprising a plurality of herpesvirus polypeptides in association with a lipid membrane, wherein the pharmaceutical composition is formed by expressing the plurality of herpesvirus polypeptides encoded by the nucleic acid in vitro in human cells from one or more nucleic acid molecules comprising a plurality of immunogen coding regions which collectively encode the plurality of herpesvirus polypeptides, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp 42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus.

Further aspects provide uses in medicine and methods of making the pharmaceutical compositions.

### BRIEF DESCRIPTION OF THE DRAWINGS

FIG. 1: Sequence alignment showing culture mutation in Domain I of glycoprotein gB structure, in HSV2 and other herpesviruses.

FIG. 2: Prophylactic HSV2 DNA vaccine trial for protection against acute infection in preclinical model, as described in Example 3. “VTL-VLMgD DNA” contains HSV2 genes in expression vectors, namely encoding gD together with mutated gB, gH, and gL. 2A: Protection from pathology, showing complete protection by VTL-VLMgD DNA. 2B: Protection from infection, showing inhibition of virus secretion post virus challenge. In VTL-VLMgD DNA immunised animals, virus secretion was undetectable by day 8 post virus challenge (0/12 animals any virus; p<0.02).

FIG. 3: Sequence alignment showing Rid1 HVEM interaction mutation in glycoprotein gD in HSV2 and other herpesviruses. Initiating methionine is +1 here and 26K+1 in mature form.

FIG. 4: Sequence alignment showing VZV fusion inhibition mutations in glycoprotein gB in HSV2 and other herpesviruses. Fusion inhibition mutations in glycoprotein B VZV structure in conserved Domain IV beta23 and beta30 folds modeled aligned in reference strains of (A.) HSV1, HSV2 and VZV for the Beta 23 fold and (B.) HSV1, HSV2, VZV, EBV, CMV, HHV6A, HHV6B, HHV7 and HHV8 for the Beta 30 fold; all indicated amino acids substituted to Ala.

FIG. 5: Sequence alignment showing HSV1 prefusion stabilising mutation in glycoprotein gB Domain III as demonstrated in HSV1 and aligned here with the human alphaherpesvirus HSV2, shown here, and VZV reference strains. The Histidine to Proline mutation at positions 516 in HSV1, 513 in HSV2, and 526 in VZV gB amino acid sequences are indicated, with the Pro substitution Asterisked.

FIG. 6: Prophylactic HSV2 DNA vaccine trial for protection against acute infection in preclinical model, as described in Example 4. “gD DNA+VLM” contains HSV2 genes in expression vectors, namely encoding gD together with mutated gB, gH, and gL. 6A: Severity of acute lesions at 4 to 14 days post virus challenge. Ratio=number of animals with lesions/cohort, lesion severity scores data points for individual animals and cohort medians are shown. Complete protection was demonstrated for both VLM formulations for all tested animals, 12 in each cohort. The positive control subunit vaccine showed only 75% protection and the negative control no vaccine, almost no protection with only 1/11 animals showing no lesions. 6B: Severity of acute virus load. Means and error bars (SEM) shown for animals with numbers with detectable viral load in each cohort of 11-12 as indicated. Both VLM formulations provided for two logs reduced virus titre at day 2 post virus challenge, and virus clearance by day 8. Virus was still detectable in the positive control subunit protein vaccine group which overall showed less virus reduction post-challenge.

FIG. 7: Prophylactic HSV2 DNA vaccine trial for protection against recurrent disease and virus reactivation in the preclinical in vivo model, as described in Example 5. 7A: Severity of recurrent lesions at 15 to 63 days post virus challenge, shown by cumulative recurrent lesion days. Data points for individual animals and cohort medians are shown. Score=number of animals with lesions/cohort. The VLM formulation combined with CCL5 shows reduced recurrent lesion days in comparison to VLM only formulation or no vaccine treatment. 7B: Protection from recurrences, as shown by cumulative lesion days for means of the animals who experienced recurrences per cohort. In the gD DNA+VLM/CCL5 formulation only half of the cohort experienced any lesion recurrence (6/12). Protection from recurrences in comparison to no vaccine treatment was only observed with the VLM and CCL5 combination, and the gD subunit protein vaccine positive control. 7C: Protection against recurrent virus shedding, as measured by quantitative DNA PCR, qPCR, with SD. Data are total recurrences in individual animals and cohort means. Score=number of animals with detectable viral DNA/cohort. Only the VLM formulation combined with CCL5 showed significant reduction in virus shedding after virus reactivation. There was no reduction with the positive control gD subunit protein vaccine. 7D: Protection against recurrent virus shedding occurrences, as measured by quantitative DNA PCR. Data are mean percent swab positive days. Score=total swab positive days for all animals in cohort/number of tests (number of days per number of animals in cohort). A trend for protection from recurrent shedding of virus was only observed with the VLM combined with CCL5 formulation. There were 180 available swabs taken for each cohort. In the no vaccine group there were 165 total swabs taken.

FIG. 8: Prophylactic HSV2 DNA vaccine trial for protection against latent infection in preclinical model, as described in Example 5. 8A: Protection against latent infection in the dorsal root ganglion (DRG), as measured by quantitative DNA PCR at day 63 post virus challenge. Data are means. Standard error bars (SEM) are indicated. All vaccine formulation provided for significant reductions in latent infection. 8B: As in 8A but data presented as number of individuals with detectable viral DNA (yes) and no detectable viral DNA (no). VLM formulations show a trend for protection from establishment of latent infection in the DRG. 8C: Protection against latent infection in the spinal cord, as measured by quantitative DNA PCR at day 63 post virus challenge. Data are means. Error bars are SEM. All vaccine formulation provided for significant reductions in latent infection. 8D: As in 8D but data presented as number of individuals with detectable viral DNA (yes) and no detectable viral DNA (no). VLM formulations significantly reduced number of animals with detectable viral DNA in the spinal cord.

FIG. 9: Prophylactic HSV2 DNA vaccine trial for protection against recurrent infection virus shedding in preclinical model, as described in Example 7 evaluating a combination immunisation of VLM and VIT. 9A: Protection against recurrent virus shedding, as measured by quantitative DNA PCR, qPCR, with SD. Data are total recurrences in individual animals and cohort means. Score=number of animals with detectable viral DNA/cohort. The VLM formulation combined with CCL5 showed significant reduction in virus shedding after virus reactivation, while that with VIT showed a trend. There was no reduction with the positive control gD subunit protein vaccine. 9B: Protection against recurrent virus shedding occurrences, as measured by quantitative DNA PCR. Data are mean percent swab positive days. Score=total swab positive days for all animals in cohort/number of tests (number of days per number of animals in cohort). A trend for protection from recurrent shedding of virus was only observed with the VLM combined with either cytokine genes, CCL5 or VIT. There were 180 available swabs taken for each cohort. In the no vaccine group there were 165 total swabs taken. 9C: Protection against cumulative recurrent virus shedding days were compared. In contrast to the subunit protein gD vaccine which actually increased the shedding days versus no vaccine treatment, only the formulations containing VLM and the cytokine genes CCL5 or VIT significantly reduced cumulative shedding days at both one and two months post virus challenge, p<0.05 indicated by asterisk.

FIG. 10: Prophylactic HSV2 DNA vaccine trial for protection against recurrent infection causing disease lesions in preclinical model, as described in Example 7 evaluating here a combination immunisation of VLM and VIT. 10A: Severity of recurrent lesions at 15 to 63 days post virus challenge, shown by cumulative recurrent lesion days. Data points for individual animals and cohort medians are shown. Score=number of animals with lesions/cohort. The VLM formulation combined with CCL5 and especially VIT show reduced recurrent lesion days in comparison to VLM only formulation or no vaccine treatment. The VLM and VIT formulation was most effective and comparable to the subunit protein. 10B: Protection from recurrences, as shown by cumulative lesion days for means of the animals who experienced recurrences per cohort. In the gD DNA+VLM/CCL5 formulation only half of the cohort experienced any lesion recurrence (6/12), while the gD DNA+VLM/VIT combination over half the cohort (7/12, 58%) were completely protected and 100% of the animals were protected from any vesicular disease. Most protection from recurrent disease in comparison to no vaccine treatment was observed with the VLM and the cytokine gene combinations, VIT, and the gD subunit protein vaccine positive control.

FIG. 11: Prophylactic HSV2 DNA vaccine trial for protection against latent infection in preclinical model, as described in Example 7 and evaluated here for combined formulation of VLM+VIT DNA immunisation. Protection against latent infection in the dorsal root ganglion (DRG), as measured by quantitative DNA PCR at day 63 post virus challenge. Data are means. Standard error bars (SEM) are indicated. All vaccine formulation provided for significant reductions in latent infection.

FIG. 12. Herpes simplex virus type 2 (HSV-2) neutralising antibody titers in guinea pig serum after 2 intramuscular immunisations with vaccine formulations containing DNA encoding gD with VLM and with DNA encoding cytokines CCL5 chemokine or VIT1, virokine immune therapeutic, compared to gD subunit protein vaccine formulation with mpl and alum, or no vaccine treatment. *** indicates p<0.001 compared to no vaccine treatment.

### DETAILED DESCRIPTION OF PREFERRED EMBODIMENTS OF THE INVENTION

The present invention has been developed using the novel strategy of presenting the immune system with herpesvirus polypeptides in the form of ‘virus-like membranes’, in which the herpesvirus polypeptides responsible for cell binding and fusion are present in native interacting conformations in association with a lipid membrane as expressed in vivo. The presence of the herpesvirus cell binding and fusion machinery in this form is believed to induce native complexes of proteins and their transition states, which mediate cellular fusion events in the immunised host. This may include receptor binding to host cells, triggering cell fusion by conformational changes in the multi-protein complex which then draws the cellular membranes into proximity together (mimicking the process of the virus and the cell membrane drawn together during infection), and induction of cell fusion between the adjacent membranes. These cell fusion events are believed to mimic virus infection of cells, and initiate innate immune mechanisms detecting cell fusion as a damage signal, which boost the capacity of adaptive immunity. Typically, the vaccine composition is provided as a polynucleotide vaccine, and the herpesvirus cell binding and fusion machinery is expressed and assembled in the cells of the host to create ‘virus-like membranes’ in vivo. The polynucleotide vaccine may incorporate a biased nucleotide composition towards increased CpG bias to induce innate mechanisms such as through the TLR9 signalling cascade to induce cellular immunity. Alternatively, ‘virus-like membranes’ may be formed in vitro and provided in a vaccine composition, such as using cellular formulations or as exosomes.

In contrast to typical approaches to polynucleotide vaccines, which attempt to improve an immune response by increasing the antigenic dosage or by means of synthetic adjuvants, our invention of ‘virus-like membrane’ vaccines places the focus on the native quality of the response and its potency via generating the ‘fusion machinery’. Further, the approach differs from subunit protein vaccine formulations in which glycoproteins are not presented in a membrane, and therefore would not be in the required fusogenic conformations.

Cell fusion mediated by herpesviruses is a required first step for infection (Hilterbrand and Heldwein, 2019). The inventor reasoned that immune antibodies to inhibit cell fusion are required to be directed to the interacting conformations of components, in transition states between pre-fusion and post-fusion complexes, which mediate this process. In herpes simplex virus, there are four essential glycoproteins, which can mediate cell fusion in in vitro cellular assays (Hilterbrand and Heldwein, 2019). These include the immunogen gD, as well as the fusogen gB and fusion regulators the gH/gL complex conserved in herpesvirus (Gompels et al., 1991; Gompels and Minson, 1989; Hilterbrand and Heldwein, 2019). Transfection of the four genes encoding these HSV1 or HSV2 glycoproteins is necessary and sufficient to result in cellular fusion (Turner et al., 1998). Although gH and gL form stable heterodimers, the interactions between the other components are transitory and trigger conformational changes between prefusion and postfusion conformations. The crystal structure of the postfusion gB conformation has been determined as reviewed (Hilterbrand and Heldwein, 2019), and a prefusion conformation determined using prefusion stabilising mutation (Vollmer et al, 2020), but the transition states are under evaluation. Although there is evidence for interactions between gD, gB and gH/gL, these interactions appear transient. Taken together the fusion complex includes the proteins necessary and sufficient to conduct fusion and may include forms both prefusion or postfusion, with variants that may stabilise either form. Therefore, the inventor reasoned that for an effective immune response, the immunised host should be exposed to these transient prefusion and transition state forms in order to block functions in cell fusion and initiation of infection as well as raise effective immunity to naturally presented conformational epitopes as well as exposed linear sites.

Polynucleotides

The first aspect of the invention provides a pharmaceutical composition comprising one or more nucleic acid molecules comprising a plurality of immunogen coding regions which collectively encode a plurality of herpesvirus polypeptides, wherein the one or more nucleic acid molecules are capable of expressing the plurality of herpesvirus polypeptides when introduced into a vertebrate cell, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus; and

wherein the pharmaceutical composition is sterile, and is provided in a sealed sterile container.

The one or more nucleic acid molecules of the pharmaceutical composition of the first aspect comprise a plurality of immunogen coding regions which collectively encode a plurality of herpesvirus polypeptides. Each immunogen coding region encodes a different herpesvirus polypeptide. Multiple, such as all immunogen coding regions, may be encoded by one nucleic acid molecule. Alternatively, at least one such as each immunogen coding region may be encoded by a separate nucleic acid molecule. Any possible combination of nucleic acid molecules comprising multiple or single encoding regions is contemplated as long as, collectively, the one or more nucleic acid molecules comprise the plurality of immunogen coding regions. The terms “nucleic acid molecule” and “polynucleotide” are used interchangeably herein and refer to a polymeric form of nucleotides of any length, either deoxyribonucleotides or ribonucleotides, or analogs thereof. The one or more nucleic acid molecules may be deoxyribonucleic acid (DNA) polynucleotides, as expressed from appropriate formulations such as synthesized expression DNA constructs, plasmid expression vectors such as bacterial plasmid expression vectors, or viral expression vectors such as adenoviral vectors; or ribonucleic acid (RNA) polynucleotides, such as synthesized RNA, or expressed from plasmid expression vectors modified by enzymes to produce the mRNA transcript, which may include modified nucleosides as described herein. This can be prepared as a one or two step reaction using a DNA plasmid to encode the RNA transcript, which is transcribed by RNA polymerase followed by enzymatic 5′ capping and 3′ polyadenylation reactions, followed by removal of double stranded RNA then formulation in a lipid carrier nanoparticle as reviewed (Kowalzik et al 2021; Rosa et al 2021) and such as disclosed in WO2019/035066 A1. Formulations may also include helper lipids as utilised in known effective mRNA vaccines for SARS-CoV2 such as 1,2-distearoyl-snglycero-3-phosphocholine (DSPC), cholesterol and/or a diffusible PEG-lipid (2-[(polyethylene glycol)-2000]-N,N-ditetradecylacetamide, PEG2000-DMA as used in the SARS-CoV2 human vaccine BNT162b2 or 1,2-dimyristoyl-rac-glycero3-methoxypolyethylene glycol-2000 and/or PEG2000-DMG as used in the SARS-CoV2 human vaccine mRNA-1273) as reviewed in (Verbeke et al 2021). Alternative RNA species include viral vectors, for example self amplifying RNA (Blakney A K, Ip S, Geall A J. An update on self-amplifying mRNA vaccine development. Vaccines (Basel). 2021 Jan. 28; 9(2):97. doi: 10.3390/vaccines9020097). The one or more nucleic acid molecules are typically of the same type, such as all DNA or all RNA, but could comprise combinations of DNA and RNA. Both DNA and RNA formulations can be administered for example by intramuscular inoculation, or other routes described herein, to generate protective immunity. Polynucleotide vaccines are demonstrated effective with the first SARS-CoV2 mRNA vaccine approved for use in humans, through intramuscular two dose vaccination with a 28 day interval, a nucleoside modified RNA encoding the Spike glycoprotein, then encoated by a lipid nanoparticle, as reviewed (Lambe 2021).

“Pharmaceutical composition” as used in relation to the first aspect of the invention is synonymous with “polynucleotide vaccine”. Both terms imply that the one or more nucleic acid molecules are isolated. The term “isolated” when used in the context of a nucleic acid molecule refers to a nucleic acid molecule that is substantially free of structures or compounds with which it is associated in its natural environment, and is thus distinguishable from a nucleic acid molecule that might occur naturally. For instance, an isolated nucleic acid molecule is substantially free of cellular material or other polypeptides or nucleic acids molecules, including herpesvirus genomic material, from viral or infected cell source from which it may be derived. Thus, the pharmaceutical composition comprises one or more nucleic acid molecules which encode specific herpesvirus polypeptides (the virus entry cell fusion complex), but not others that would also be needed to cause a viral infection.

A nucleic acid sequence, which “encodes” a selected polypeptide is a nucleic acid molecule which is transcribed (in the case of DNA) and translated (in the case of mRNA) into a polypeptide in vivo when placed under the control of appropriate regulatory sequences. By ‘immunogen coding region’ is meant an open reading frame (ORF) encoding the immunogen, typically also comprising a 5′ Kozak sequence operably linked to the ORF. The boundaries of the open reading frame are determined by a start codon at the 5′ (amino) terminus and a translation stop codon at the 3′ (carboxy) terminus. By ‘immunogen’ is meant the encoded herpesvirus polypeptide that is expressed in vivo following administration of the pharmaceutical composition. The one or more nucleic acid molecules are capable of expressing the plurality of herpesvirus polypeptides when introduced into a vertebrate cell. This may be achieved by virtue of the following features. In the case of a DNA molecule, the immunogen coding region is operably linked to a 5′ promoter which is capable of driving transcription of the immunogen coding region in the vertebrate cell, such as via promoting binding of RNA polymerase II. The immunogen coding region is typically contiguous with a 3′ untranslated region comprising 3′ polyadenylation sequences, and terminating with a transcription termination sequence. In the case of an RNA molecule, the immunogen coding region is typically contiguous with a 3′ untranslated region comprising 3′ polyadenylation sequences, and terminating with a transcription termination sequence. The capability for expression in a vertebrate cell typically relates to expression in a cell of a vertebrate species for which the pharmaceutical composition is intended, typically a mammal, typically a human. Expression of herpesvirus polypeptides in cells may be detected in vitro by means known to the skilled person. A suitable method to detect the polypeptide expressed in cells could use assays for polykaryocyte formation or using immunofluorescence or Western blots (Muggeridge, 2000; Rogalin and Heldwein, 2016; Turner et al., 1998)

According to the first aspect, each of the plurality of immunogen coding regions has at least 90% sequence identity to a native coding region for a corresponding native full-length herpesvirus polypeptide from the same herpesvirus species. The relatedness between two nucleotide sequences (or between two amino acid sequences) is described by the parameter “sequence identity”.

Suitably, each of the plurality of immunogen coding regions possesses at least 95% sequence identity, such as at least 97% sequence identity, at least 99% sequence identify, at least 99.5% sequence identify or 100% sequence identity to the native coding region for the corresponding native full-length herpesvirus polypeptide. This level of sequence identity may be seen across the full length of the relevant SEQ ID NO sequence. Thus, the immunogen coding region typically exhibits at least 95% sequence identity against the native coding region for the corresponding native full-length herpesvirus polypeptide, by virtue of itself being full-length or having a length of alignment of at least 95% against the native coding region of the the full-length herpesvirus polypeptide. Typically, the length of alignment is at least 96%, 97%, 98%, 99%, 99.5% or is 100%. Typically, the length of alignment is 100%, and the sequence identity is at least 96%, 97%, 98%, 99%, 99.5% or is 100%.

Suitably, each of the plurality of immunogen coding regions encodes a herpesvirus polypeptide having at least 90% sequence identity to a corresponding native full-length herpesvirus polypeptide encoded by a native coding region from the same herpesvirus species. Suitably, the level of sequence identity between the herpesvirus polypeptide and the corresponding native full-length herpesvirus polypeptide is at least 96%, 97%, 98%, 99%, 99.5% or is 100%. This level of sequence identity may be seen across the full length of the relevant SEQ ID NO sequence. Thus, the herpesvirus polypeptide typically exhibits at least 90% sequence identity against the corresponding native full-length herpesvirus polypeptide, by virtue of itself being full-length or having a length of alignment of at least 90% against the native full-length herpesvirus polypeptide. Typically, the length of alignment is at least 96%, 97%, 98%, 99%, 99.5% or is 100%. Typically, the length of alignment is 100%, and the sequence identity is at least 96%, 97%, 98%, 99%, 99.5% or is 100%.

It is believed that the presence of the transmembrane domain in the herpesvirus polypeptides which, in their native form, possess a transmembrane domain, is important. The presence of the transmembrane domain, such as in variants of a herpesvirus polypeptide, can be predicted by methods known in the art for example reviewed (Nugent and Jones, 2009) In the case of HSV1 and HSV2, each of gB, gH and gD comprise a transmembrane domain. In order to associate with the membrane or be secreted gB, gH, gD and gL all comprise N-terminal signal sequences which enable their insertion in the rough endoplasmic reticulum, RER, with co-translation into the lumen of the RER and subsequent cleavage of the signal sequence by the signal recognition particle proteolytic processing. The ability of the N-terminal sequence of a herpesvirus polypeptide, such as a variant, to act as a signal sequence can be predicted by methods known in the art (Nugent and Jones, 2009). Thus, each of the gB, gH, gD and gL encoded by the immunogen coding regions should comprise a functional N-terminal signal sequence. The gB, gH and gD encoded molecules have a second transmembrane domain that allows anchoring within the membrane together with cytosolic exposed and positively charged stop anchor sequences that allow embedding in the membrane. While the expressed gL is essentially a secreted protein, and lacks a transmembrane domain, it is membrane associated via its heterodimer formation with gH. All of the native HSV1 and HSV2 gB, gH, gD and gL glycoproteins are glycosylated post-translationally during embedding in the membrane, and via processing through the exocytic pathway and the glycosylation may be important for conformation and function. It is preferred that the herpesvirus polypeptides encoded by the immunogen coding regions comprise the native glycosylation sites. Glycosylation is either N-linked or O-linked and the consensus sequences are mainly NXT/S or sites on T,S respectively as reviewd (Hamby and Hirst, 2008). The native HCMV gB comprise a palmitoylation site, which increases cell fusion at C777 (strain VR1814) (Patrone et al., 2016) and in the Merlin reference strain C779. It is preferred that, where present in the native polypeptide, the polypeptide encoded by the immunogen coding region e.g. gB comprises a palmitoylation site.

The gD glycoprotein binds to the TNF-receptor superfamily LIGHT and act as check point inhibitor giving a natural boost to immune responses as blocking the negative regulator system (Cai and Freeman, 2009). Therefore, if this receptor ligand relationship is maintained this can increase types of immune responses. The interacting domain has been mapped to the external part of the molecule, but correct folding of the membrane-tethered form as expressed by the whole gene can facilitate interactions between the interacting immune cell and the glycoprotein expressing cells (Cairns et al., 2019; Lu et al., 2014). It is therefore preferred that the gD maintains the ability to bind to TNF-receptor superfamily LIGHT, as described in Cai and Freeman, 2009.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the −nobrief option) is used as the percent identity and is calculated as follows:

(Identical Residues×100)/(Length of Alignment−Total Number of Gaps in Alignment)

A “variant” refers to a protein wherein at one or more positions there have been amino acid insertions, deletions, or substitutions, either conservative or non-conservative. A “variant” may have modified amino acids. However, as noted above, it is preferred that variants retain the same N-linked and O-linked glycosylation sites and, where present, palmitoylation site as the native protein.

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labelled “longest identity” (obtained using the −nobrief option) is used as the percent identity and is calculated as follows:

(Identical Deoxyribonucleotides×100)/(Length of Alignment−Total Number of Gaps in Alignment)

The corresponding (or reference) native coding region to which the immunogen coding region is compared is from the same herpesvirus species, typically from the same herpesvirus strain. In the alternative, the corresponding (or reference) native full-length herpesvirus polypeptide to which the herpesvirus polypeptide is compared is encoded by a native coding region from the same herpesvirus species, typically from the same herpesvirus strain. The plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus.

In any given herpesvirus species, the above four polypeptides, when appropriately assembled in association with a lipid membrane, form the fusogenic complex of the herpesvirus. In other words, the one or more nucleic acid molecules are capable of expressing the plurality of herpesvirus polypeptides in the form of a herpesvirus fusion complex when introduced into the vertebrate cell. By “fusogenic complex”, or “fusion complex” we mean that the polypeptides, when co-expressed in a vertebrate cell, are necessary and sufficient for cell fusion. The fusion complex is formed by the core fusion mediators gB, gH, gL together with a cell binding glycoprotein component as disclosed in (i) above. Thus, for a strain of HSV2, gB, gH, gL and gD of the HSV2 strain are required. For a strain of VZV, gB, gH, gL and either of gE or gI of the VZV strain are required. By “gB, gH, gL of the respective cognate human herpesvirus” we mean that gB, gH and gL herpesvirus polypeptides mentioned in (ii) above are derivable from the same herpesvirus species, typically from the same herpesvirus strain as the herpesvirus polypeptide selected from (i) above. Thus, where the first polypeptide is derivable from gD of herpes simplex virus 2, the gB, gH and gL are also typically derivable from the same strain of herpes simplex virus 2. Where the first polypeptide is derivable from gD of herpes simplex virus 1, the gB, gH and gL are also typically derivable from the same strain of herpes simplex virus 1. By “derivable”, we mean that the immunogen coding region has at least 90% sequence identity to a native coding region for a corresponding native full-length herpesvirus polypeptide; or that the polypeptide has at least 95% sequence identity to a corresponding native full-length herpesvirus polypeptide. Therefore, it is not necessarily the case that each of the four herpesvirus polypeptides are identical to a herpesvirus polypeptide from the same strain. By “gO selected from genotypes 1-8 of human cytomegalovirus”, we refer to the genotypes known in the art as gO1a, gO1b, gO1c, gO2a, gO2b, gO3, gO4, gO5a/gO5, such as those provided in Table 1.

Where alternatives exist for one of the polypeptides of the fusion complex, the one or more nucleic acid molecules may encode one or more than one of such polypeptides. Thus, for a strain of VZV, gB, gH, gL and either or both of gE and gI of the VZV strain may be encoded by and expressible by the one or more nucleic acid molecules. In embodiments, the pharmaceutical composition comprising the one or more nucleic acid molecules does not comprise a nucleic acid molecule encoding or capable of expressing a herpesvirus polypeptide other than (i) those that form the fusion complex, which is the core fusion mediators gB, gH, gL together with a cell binding glycoprotein component as disclosed herein, accepting that where there are cell binding glycoprotein alternatives, one or more than one of the alternatives may be present; and optionally (ii) a viral immunomodulator, typically a secreted viral immunomodulator, such as a chemokine as disclosed herein. Thus, the herpesvirus polypeptides encoded by the one or more nucleic acid molecules may be limited to those forming the fusion complex, and optionally also a herpesvirus immunomodulator.

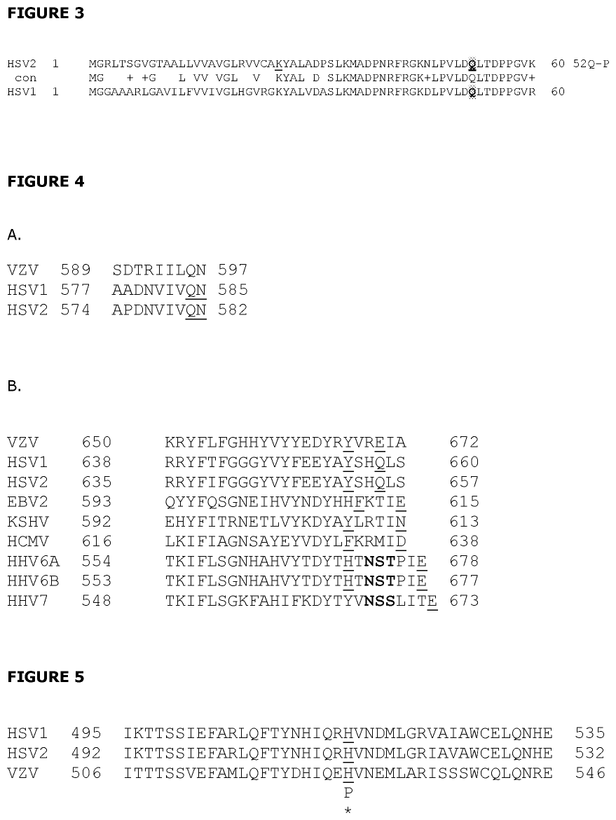

HSV2 or HSV1 gD, although not a direct fusion protein, triggers the fusion machine and also serves to boost immune responses as a native immune check point inhibitor by virtue of binding to its receptors (preventing BTLA binding CD160-HVEM) (Lasaro et al., 2005; Zhang and Ertl, 2014) and also via interacting with dendritic cell subsets (Porchia et al., 2017). The gD molecules also bind to a second receptor nectin and interaction with respective ligands can affect the nature of the immune response. For example, lowering or removing interaction with HVEM can increase IgG2 ADCC, antibody dependent cellular cytoxicity (Burn Aschner et al., 2020), while retaining this interaction can increase IgG1 neutralising antibodies. A SNP variant modification can reduce the interaction with HVEM as shown in strain ANG and Rid1 variant virus (Montgomery et al., 1996) and this is introduced in HSV2 gD in SEQUENCE ID65 and in reference HSV1 gD in SEQUENCE ID63. Therefore utilising wild type gD2 can stimulate neutralising antibodies as demonstrated, with utility for other IgG1 responses useful for controlling for example HIV1 (Kadelka et al., 2018), and avoiding ADCC, important where antibody dependent enhancement, ADE, reactions could be harmful by increasing infections such as known for Dengue and Coronaviruses, so utility for targeting these viruses (S. et al., 2020). While mutated SNP gD2, with reduced interaction with HVEM, has utility for stimulating IgG2 ADCC responses with utility for controlling HSV infections. This approach also has utility for therapeutic vaccines for example where individuals are already seropositive for HSV then to provide the DNA vaccine with VLM with variant gD encoded to boost ADCC as shown for HSV2 deleted in gD used as an immunogen (Burn Aschner et al., 2020). This is based on a variant SNP in Rid1 HSV1 gD and from deletion studies on HSV2 gD. Here the SNP is transposed to reference HSV1 strain 17 and HSV2 gD through alignment analyses (FIG. 3, TABLE 4; SEQUENCE IDs 63 and 65; and translated polypeptide sequences SEQ ID NOs. 64 and 66). Suitably, the gD polypeptide encoded by the immunogen coding region for gD comprises a mutation which lowers interaction with the HVEM receptor, such as a substitution at a position corresponding to position 52 of SEQ ID 64.

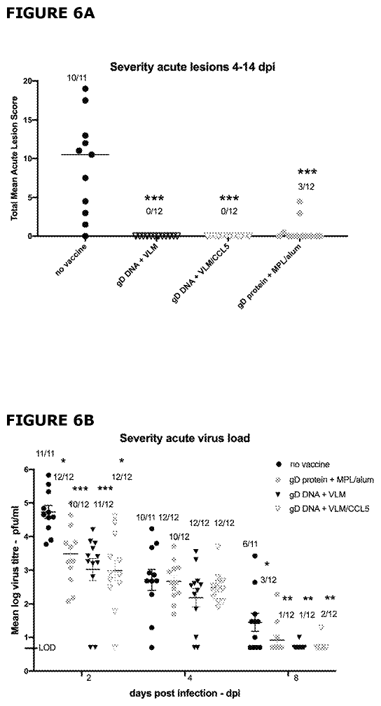

Similarly to the SNPs in gD, a SNP variant in HSV2 gB can also redirect the immune responses, in this case to an early prefusion form of the glycoprotein, as indicated in SEQUENCE IDs 71-74. This is based on studies on VZV gB and shown here for HSV2 gB using alignment analyses (FIG. 4, TABLE 4) then SNP mutations of the amino acid codons in all representative human herpesvirus encoded gB molecules as in SEQUENCE ID 67-70. The wild type encoded amino acids are demonstrated in conserved structural folds of gB, in domain DIV beta23 and beta30 (Oliver et al., 2020).

The first set of prefusion like gB variants are conserved in alphaherpesviruses in Domain DIV beta23 fold as shown in FIG. 4A and represented by SEQUENCE IDS 67, 68, 71, 72, 75, and 76. The second set of prefusion like gB variants are conserved in human alpha, beta and gammaherpesvirus as shown in Domain DIV beta30 fold in FIG. 4B, TABLE 4 and represented as encoded by SEQUENCE IDs 69, 79, 73, 74, 77, 78 and 79-90. These variants can form the complex of membrane associated glycoproteins, but do not perform cell fusion therefore would evade innate signalling via TLR7 and have utility in presenting epitopes for stimulating antibody generation to inhibit transition to cell fusion, which is required for cell infection. They would retain the ability to be expressed endogenously to also stimulate cellular immunity via antigen presentation via MHC molecules on the cell surface.

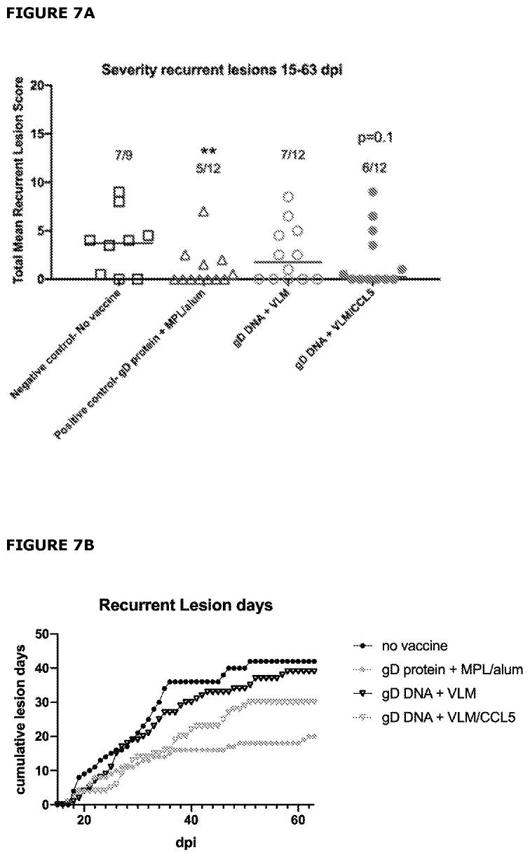

Further distinct prefusion stabilising mutations are shown for SNPs in gB in FIG. 5, TABLE 4 as disclosed for HSV1 (Vollmer et al, 2020) and represented by SEQUENCE IDS 131, 132 and 133 including HSV2 and VZV gB SNPs identified through alignments herein (FIG. 5).

Suitably, the gB polypeptide encoded by the immunogen coding region for gB comprises a mutation which stabilises gB in a trimer in the prefusion conformation, such as a mutation in the gB structure domains III and/or IV, such as a substitution at a position corresponding to one or more of the substitutions in SEQ IDs 67 to 90 or 132 to 134.

Typically, the ability of the native polypeptides (or polypeptide encoded by the immunogen encoding region) to mediate cell fusion is assayed in a vertebrate cell of the same species for which the pharmaceutical composition is intended, typically a mammalian species, typically a human. A method for detecting cell fusion resulting from the co-expression of the polypeptides of a fusogenic complex is described in Turner et al., 1998. Cell fusion may be detected by the formation of polykaryocytes, i.e. cells possessing more than one cell nucleus. For example, monolayers of Cos cells may be transfected with plasmids from which the polypeptides of the fusogenic complex are expressed, overlayed with VERO cells or other permissive cell types, and polykaryoctes detected by nuclear staining. Cell fusion is deemed to have occurred where the number of polykaryoctes having a minimum number of nuclei, such as 10, is greater following transfection with plasmids from which the polypeptides of the fusogenic complex are expressed, compared to mock-transfected cells. Typically, the number of polykaryocytes may be at least 2 times, such as at least 5 times, such as at least 10 times as many following transfections with plasmids from which the polypeptides of the fusogenic complex are expressed. Similarly, using this same assay, these fusion effects can be compared to mutations or variants of these proteins that can stabilise fusion forms, for example prefusion stabilising mutations would arrest the fusogenic transition and lower polykaryocyte formation.

Preferably, the reference herpesvirus strain to which the herpesvirus polypeptides encoded by the immunogen coding regions are compared is a clinical isolate representative of a prevalent strain of the virus found in an infected population, typically an infected human population. Isolated strains, and the relevant nucleic acid sequence, may be subjected to deep next generation sequencing so any effect of populations of strains can be controlled and the representative dominant wild type sequence identified. Clinically prevalent herpesvirus strains, their genome sequences, and the coding nucleic acid sequences of the defined polypeptides are known in the art. Representative examples are included in Table 1. The polypeptide sequences themselves are also known, and obtainable by translation of the open reading frames of the nucleic acid sequences.

TABLE 1
Reference HHV species and strains with relevant nucleic acid
sequences with accessions Nos and variants with sequence IDs.

| | Exemplary strain | | |
|---|---|---|---|
| | reference | | |
| | sequence | | |
| | genome, | | |
| HHV | or gene NCBI | Encoded | Coding nucleic acid sequence, |
| species | accession No. | polypeptide | Accession Nos. |
| HSV1 | Strain17, | gB | gB, UL27, NC_001806.2: c55795-53081 |
| | NC_001806.2 | gH | gH, UL22, NC_001806.2: c46383-43867 |
| | | gL | gL, UL1, NC_001806.2: 9338-10012 |
| | | gD | gD, US6, NC_001806.2: 138423-139607 |
| HSV2 | HG52, | gB | gB, UL27, NC_001798.2: c56152-53438 |
| | NC_001798 | gH | gH, UL22, NC_001798.2: c46570-44054 |
| | | gL | gL, UL1, NC_001798.2: 9463-10137 |
| | | gD | gD, US6, NC_001798.2: 141016-142197 |
| VZV | Dumas, | gB | gB, ORF31, NC_001348.1: 56819-59614 |
| | NC_001348.1 | gH | gH, ORF37, NC_001348.1: 66074-68599 |
| | | gL | gL, ORF60, NC_001348.1: c101649-101170 |
| | | gE | gE, ORF67, NC_001348.1: 114496-115560 |
| | | gI | gI, ORF68, NC_001348.1: 115808-117679 |
| EBV- | B958 and RAJI | gB | gB, BALF4, NC_007605.1: c158864-156291 |
| type1 | combination, | gH | gH, BXLF2, NC_007605.1: c130747-128627 |
| | NC_007605.1 | gL | gL, BZLF2, NC_007605.1: c89828-89157 |
| | | gp350 | gp350, BLLF1, NC_007605.1: c79865-77142 |
| | | gp42 | gp42, ZBLF2, NC_007605.1: c89828-89157 |
| EBV- | AG876, | gB | gB, BALF4, NC_009334.1: c160348-157775 |
| type2 | NC_009334.1 | gH | gH, BXLF2, NC_009334.1: c131574-129454 |
| | | gL | gL, BKRF2, NC_009334.1: 98500-98913 |
| | | gp350 | gp350, BLLF1, NC_009334.1: c79936-77276 |
| | | gp42 | gp42, BZLF2, NC_009334.1: c90630-89959 |
| HCMV | Merlin, | gB | gB, UL55, NC_006273.2: c84789-82066 |
| | NC_006273.2 | gH | gH, UL75, NC_006273.2: c111452-109224 |
| | | gL | gL, UL115, NC_006273.2: c165858-165022 |
| | | gO (gO5) | gO5, UL74, NC_006273.2: c108848-107430 |
| HCMV | AD169 | gO1a | gO1, UL74, NC_001347.5: c108456-107056 |
| | DM7 | gO1b | gO1b, UL74, AF531334.1 |
| | Toledo | gO1c | gO1c, UL74, AF531355.1 |
| | PH-BAC | gO2a | gO2a, UL74, AC146904.1: 119468-120856 |
| | SW1715 | gO2b | gO2b, UL74, AF531342.1 |
| | SW475 | gO3 | gO3, UL74, AF531348.1 |
| | Towne | gO4 | gO4, UL74, AF531356.1 |
| | K141 | gO5a | gO5a, UL74, EU686518, an internal initiation |
| HHV-6A | U1102, | gB | gB, U39, NC_001664.4: c62129-59637 |
| | NC_001664.4 | gH | gH, UL48, NC_001664.4: c80170-78086 |
| | | gL | gL, UL82, NC_001664.4: c123457-122705 |
| | | gO | gO, U47, NC_001664.4: c77919-75964 |
| | | gOvariant-A | SEQUENCE ID 12 |
| HHV-6B | Z29, | gB | gB, U39, NC_000898.1: c63200-60708 |
| | NC_000898.1 | gH | gH, U48, NC_000898.1: c81349-79265 |
| | | gL | gL, U82, NC_000898.1: c124745-123993 |
| | | gO | gO, U47, NC_000898.1: c78999-76783 |
| | | gOvariant-B | SEQUENCE ID 13 |
| HHV-7 | RK, | gB | gB, U39, NC_001716.2: c61093-58625 |
| | NC_001716.2 | gH | gH, U48, NC_001716.2: c77113-75041 |
| | | gL | gL, U82, NC_001716.2: c121009-120269 |
| | | gO | gO, U47, NC_001716.2: c74803-73862 |
| KSHV | GK18, | gB | gB, ORF8, NC_009333.1: 8665-11202 |
| | NC_009333.1 | gH | gH, NC_009333.1: 37212-39404 |
| | | gL | gL, NC_009333.1: c70014-69511 |
| | | gK8.1, gp55 | K8.1, NC_009333.1: 76014-76437, 76532-76794 |
| | | gK8.1variant | SEQUENCE ID 14 |
| KSHV | BCBL-1 | gK8.1A | gK8.1A, AF068829 |
| | | gK8.1B | gK8.1B, AF068830.1 |

The herpesvirus polypeptides encoded by the immunogen coding regions are suitably all derived from the same herpesvirus strain, in the sense that they are substantially identical to the corresponding native full-length herpesvirus polypeptide encoded by a single strain. The derivation from a single strain may promote the formation of the native conformations of the polypeptides in association with a lipid membrane when expressed in a host cell. This also may aid the natural formation of the native fusogenic complex, as evidence suggests glycoproteins from different strains combine with different properties. However, the herpesvirus polypeptides encoded by the immunogen coding regions may alternatively be derived from different herpesvirus strains within the same herpesvirus species. For example, as shown in Table 1, there are nine variants of HCMV gO. A gO derived from one strain may be combined with gB, gH and gL from another. As the coding sequences of gB, gH and gL are similar between different strains, each of the plurality of immunogen coding regions encoding a herpesvirus polypeptide would still have at least 95% sequence identity to a corresponding native full-length herpesvirus polypeptide encoded by a native coding region from the same herpesvirus strain. Likewise, there are several variants of KSHV gK8.1. A gK8.1 from one strain could be combined with gB, gH and gL from another KSHV strain. For avoidance of doubt, where gO of HCMV is referred to, the gO may be any of the variants of gO that exist in different HCMV strains. gO of HHV6A should be interpreted accordingly, as encompassing any of the variants of gO that exits in different HHV6A strains; and gO of HHV6B and K8.1 of KSHV should be interpreted accordingly.

The Examples used wild type sequences from a human population HSV2 native strain, the reference sequence. All genes had wild type sequences from the same reference strain as demonstrated in human populations (Szpara et al., 2014). These had all been subjected to deep next generation sequencing so any effect of populations of strains could be controlled and the representative dominant wild type sequence synthesized, (reference human alphaherpesvirus 2, HSV2, strain HG52, NCBI accession number NC_001798). All releases of sequences were checked in NCBI Genbank (releases 7-2019 to 7-2020) and in the updated NGS deep sequenced reference to validate any mutation, with relevant sequence corrected.

Codon Usage, CpG Bias and G+C Content

In an embodiment, each of the plurality of immunogen coding regions has a codon usage, a CpG bias and/or a G+C content which is substantially the same as the codon usage, CpG bias and/or G+C content of the native coding region for the corresponding native full-length herpesvirus polypeptide. Typically, the codon usage, a CpG bias and G+C content are the same for the native coding region for the corresponding native full-length herpesvirus polypeptide as for the whole herpesvirus genome. However, for CMV, HHV-6A/B and HHV-7, only one region of the genome is CpG suppressed, and this is outside of the fusion complex genes described herein. Human genes have CpG suppression, this dinucleotide is uncommon in vertebrate coding sequences, due to the mutagenic effect of C methylation followed by deamination to T residues, therefore CpG mutate to TpG, or the complement CpA, while herpesvirus have different CpG suppression in relation to latent sites of infection. Alphaherpesvirus, HHV1, HHV2 and HHV3 are not CpG suppressed, Betaherpesvirus, HHV5, HHV6A, HHV6B are largely not CpG suppressed, except in immediate early genes, while Gammaherpesvirus, HHV4 and HHV8 are CpG suppressed (Honess et al., 1989). In addition, HHV1 and HHV2 have increased CpG prevalence given their increased C+G compositions in excess of human genes. The G+C content varies in the respective genomes with medians of 67.5% HHV1, 70% HHV2, 46% HHV3, 55.4% HHV4, 57.3% HHV5, 42.4% HHV6A, 42.8% HHV6B, 36.2% HHV7, 53.8% HHV8 compared to median for human genes 41%.

Codon usage. Previous studies had focused to optimise codon usage for increased antigen production. However, recent data shows this can alter polypeptide folding (Athey et al., 2017). Correct folding of the polypeptides of the fusogenic complex is needed for regulation of pre-fusion and post-fusion states as well as transition states, and are integral to function. Various species exhibit particular bias for certain codons of a particular amino acid. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which DNA codons encode which amino acids is reproduced herein as Table 2. A corresponding genetic code applies to RNA codons, with the exception that uracil (U) is found in RNA in place of thymine (T). As a result, many amino acids are designated by more than one codon. This degeneracy allows for polynucleotide base composition to vary over a wide range without altering the amino acid sequence of the proteins encoded by the polynucleotide.

TABLE 2
Standard genetic code

| Amino acid | DNA codons |
|---|---|
| Ala, A | GCT, GCC, GCA, GCG |
| Arg, R | CGT, CGC, CGA, CGG, AGA, AGG |
| Asn, N | AAT, AAC |
| Asp, D | GAT, GAC |
| Asn or Asp, B | AAT, AAC, GAT, GAC |
| Cys, C | TGT, TGC |
| Gln, Q | CAA, CAG |
| Glu, E | GAA, GAG |
| Gln or Glu, Z | CAA, CAG, GAA, GAG |
| Gly, G | GGT, GGC, GGA, GGG |
| His, H | CAT, CAC |
| START | ATG |
| Ile, I | ATT, ATC, ATA |
| Leu, L | CTT, CTC, CTA, CTG, TTA, TTG |
| Lys, K | AAA, AAG |
| Met, M | ATG |
| Phe, F | TTT, TTC |
| Pro, P | CCT, CCC, CCA, CCG |
| Ser, S | TCT, TCC, TCA, TCG, AGT, AGC |
| Thr, T | ACT, ACC, ACA, ACG |
| Trp, W | TGG |
| Tyr, Y | TAT, TAC |
| Val, V | GTT, GTC, GTA, GTG |
| STOP | TAA, TGA, TAG |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis.

Codon usage tables for a given organism are readily available, for example, at the “Codon Usage Database” available at www.kazusa.or.jp/codon/. A comprehensive analysis of codon usage in 43 herpesviruses for which the whole genome has been sequences is found in Fu, M. Codon usage bias in herpesvirus. Arch Virol 155, 391-396 (2010); https://doi.org/10.1007/s00705-010-0597-0. The codon usage table for HHV2 is reproduced below as Table 3.

By utilizing these or similar tables, one of ordinary skill in the art can apply the frequencies to any given polypeptide sequence. Differences in codon usage between sequences may be expressed as difference in percentage frequency of a given codon for a given amino acid between the two sequences. For example, Gly might be encoded by GGC in one sequence at a frequency of 30%, but in a different sequence at a frequency of 20%. The difference in frequency is 10%. The difference in frequency for each of the 64 codons may be determined for the two sequences, and the mean taken as the mean frequency difference. By codon usage which is “substantially the same” as the codon usage of the native coding region for the corresponding native full-length herpesvirus polypeptide, we mean that the mean frequency difference between codons in the immunogen coding region and the native coding region is less than 5%, such as less than 2%, less than 1%, less than 0.5%, less than 0.1%. For example, in Table 3 compare HHV1 to HHV2 codon usage and Gly encoded by GGC is 34% in HHV2 and 29% in HHV1. Preferably, the codon usage is identical to that of the native coding region of the herpesvirus polypeptide.

TABLE 3
Codon usage table for HHV2, above panel, compared to HHV1, lower panel
fields: [tripler] [anuno acid] [fraction] [frequency; per thousand] ([number])

| | | | |
|---|---|---|---|
| UUU F 0.4 [symbol] 14.5 (14 [symbol] ) | UCU S 0.0 [symbol] 3.5 ( [symbol] 4) | UAU Y 0.17 4.7 (47 [symbol] ) | UGU C 0. [symbol] 2 3.5 (355) |
| UUC F 0. [symbol] 4 16.9 (1699) | UCC S 0.32 20.1 (202 [symbol] ) | UAC Y 0. [symbol] 23.4 (2 [symbol] 5 [symbol] ) | U [symbol] C C 0.78 12.7 (1278) |
| UUA L 0.01 1.2 (125) | UCA S 0.02 1.0 (10 [symbol] ) | UAA [symbol] 0.33 0. [symbol] (66) | UGA [symbol] 0.26 0. [symbol] ( [symbol] 1) |
| UUG L 0.07 [symbol] .3 (634) | UCG S 0. [symbol] 8 17. [symbol] (1735) | UAG [symbol] 0.41 0.8 ( [symbol] 2) | UGG [symbol] 1.00 11. [symbol] (1 [symbol] 4) |
| CUU L 0.05 4.8 (4 [symbol] 1) | CCU [symbol] 0.05 4. [symbol] (4 [symbol] ) | CAU H 0.15 3.8 (3 [symbol] 7) | CGU [symbol] 0.05 4.5 (450) |
| CUC L 0.25 22.5 (227 [symbol] ) | CCC [symbol] 0.52 4 [symbol] (4 [symbol] 96) | CAC H 0.85 21.5 (21 [symbol] 5) | CGC [symbol] 0.52 [symbol] 5.4 (457 [symbol] ) |
| CUA L 0.04 4.0 ( [symbol] 02) | CCA [symbol] 0.05 4. [symbol] (4 [symbol] 3) | CAA Q 0.15 4.2 (42 [symbol] ) | [symbol] GA [symbol] 0.06 5.7 (576) |
| CUG L 0.57 51.2 (5155) | CCG [symbol] 0.37 33.7 (339 [symbol] ) | CAG Q 0.85 23.6 (2383) | [symbol] GG [symbol] 0.30 [symbol] 6.7 (2689) |
| AUU I 0.13 [symbol] .7 (371) | ACU T 0.03 1.7 (16 [symbol] ) | AAU [symbol] 0.11 2. [symbol] (250) | AGU [symbol] 0.04 2.2 (225) |
| AUC I 0.77 22. [symbol] (2263) | ACC T 0.51 30.2 (304 [symbol] ) | AAC [symbol] 0. [symbol] 9 19.4 (195 [symbol] ) | AGC [symbol] 0.29 17.8 (1798) |
| AUA I 0.10 3.1 (30 [symbol] ) | ACA T 0.0 [symbol] 2.8 (2 [symbol] 7) | AAA [symbol] 0.21 4.1 (41 [symbol] ) | AGA R 0.02 1.5 (148) |
| AUG [symbol] 1.00 17.8 (1797) | ACG T 0.41 24.4 (2457) | AAG [symbol] 0.79 15.2 (1534) | AGG R 0.05 4.2 (421) |
| GUU V 0.1 [symbol] 6.7 (671) | GCU A 0.04 5.5 (553) | GAU D 0.14 7. [symbol] (7 [symbol] ) | GGU G 0.06 4.4 (448) |
| GUC V 0.39 [symbol] 7.0 (2716) | GCC A 0. [symbol] 4 71.1 (7167) | GAC D 0. [symbol] 44.2 (4455) | G [symbol] C G 0.44 34.1 (34 [symbol] ) |
| GUA V 0.04 [symbol] .0 (2 [symbol] ) | GCA A 0.0 [symbol] 4.6 (45 [symbol] ) | GAA [symbol] 0.17 8.1 (819) | GGA G 0.09 7.1 (717) |
| GUG V 0.48 33.1 (333 [symbol] ) | GCG A 0.39 [symbol] 1.2 (5157) | GAG [symbol] 0.83 40.3 (4065) | GGG G 0.41 32.2 (3244) |
| UUU F 0.52 15.0 (5351) | UCU S 0.07 4 [symbol] (1537) | UAU Y 0.20 5.3 (1 [symbol] 71) | UGU C 0.39 6.8 (2429) |
| UUC F 0.4 [symbol] 14.0 (4985) | UCC S 0.30 19.0 (6752) | UAC Y 0. [symbol] 0 20.9 (7436) | UGC C 0.61 10.9 ( [symbol] 875) |
| UUA L 0.03 2.3 (831) | UCA S 0.04 2.2 (7 [symbol] ) | UAA [symbol] 0.29 0.9 (320) | UGA [symbol] 0. [symbol] 0.7 (258) |
| UUG L 0.12 11.2 (3986) | UCG S 0.25 15.9 (5664) | UAG [symbol] 0.4 [symbol] 1.5 (5 [symbol] 1) | UGG [symbol] 1.00 11.5 (40 [symbol] ) |
| CUU L 0.10 9.6 (3412) | CCU P 0.07 6.9 (2470) | CAU [symbol] 0.32 8.9 (3152) | CGU [symbol] 0. [symbol] 3 [symbol] .7 ( [symbol] 10 [symbol] ) |
| CUC L 0. [symbol] 0 19.2 (6817) | CCC P 0.54 53.0 (188 [symbol] 1) | CAC [symbol] 0.58 19.2 (6833) | CGC [symbol] 0.46 34.7 (1 [symbol] 65) |
| CUA L 0.0 [symbol] [symbol] .4 (1554) | CCA P 0.12 11.4 (4059) | CAA Q 0.25 7.6 (2702) | CGA [symbol] 0.09 6.8 (2432) |
| CUG L 0.50 4 [symbol] .0 (16721) | CCG P 0.27 26. [symbol] (9251) | CAG Q 0.75 23.1 ( [symbol] 1 [symbol] ) | CGG [symbol] 0.26 19.5 ( [symbol] 50) |
| AUU I 0.27 9.2 (32 [symbol] 4) | ACU T 0.05 3.8 (1357) | AAU [symbol] 0.12 2.7 (950) | AGU S 0.05 3.4 (1 [symbol] 97) |
| AUC I 0. [symbol] 1 21.1 (7526) | ACC T 0.55 41.9 (14913) | AAC [symbol] 0. [symbol] 19.1 (6794) | AGC S 0.29 18.3 ( [symbol] 505) |
| AUA I 0.12 4.2 (1485) | ACA T 0.09 7.0 (24 [symbol] ) | AAA [symbol] 0.3 [symbol] 5. [symbol] (2058) | AGA [symbol] 0.02 1.8 (625) |
| AUG K 1.00 19.5 ( [symbol] 93 [symbol] ) | ACG T 0.30 22. [symbol] (812 [symbol] ) | AAG [symbol] 0. [symbol] 7 11.6 (4124) | AGG [symbol] 0.04 3.3 (1167) |
| GUU V 0.15 [symbol] 0.6 ( [symbol] 781) | GCU A 0.05 6.0 (2130) | GAU D 0.16 7.3 (2600) | GGU G 0.10 8.0 (2 [symbol] 3) |
| GUC V 0.35 25.4 ( [symbol] 03 [symbol] ) | GCC A 0.56 61.3 ( [symbol] 1 [symbol] 07) | GAC D 0.84 39.0 (13 [symbol] 1) | GGC G 0.36 29.1 (10342) |
| GUA V 0.10 7.1 (2532) | GCA A 0.07 7.5 (2572) | GAA [symbol] 0.23 10. [symbol] (3 [symbol] 9) | GGA G 0.10 7.9 (2 [symbol] 22) |
| GUG V 0.40 2 [symbol] .7 (10220) | GCG A 0.32 35.2 (12529) | GAG [symbol] 0.77 [symbol] .1 (12840) | GGG G 0.45 36.2 (12887) |

[symbol]

indicates data missing or illegible when filed

The different codon usage tables generated for each herpesvirus can be used to apply to different herpesvirus genes. For example, HSV have high G+C bias and no CpG suppression and therefore can be sensed by the TLR9 receptor to stimulate innate immunity. This codon usage table can be similarly applied to other herpesvirus genes which do not have this composition. Similarly HHV-6A,B and HHV-7 have low G+C bias and some regions of the genome are CpG suppressed, these codon usage tables can be used to create genes in other herpesvirus in order to avoid TLR9 receptor innate immunity signalling, while enabling evasion of detection of ZAP, zinc finger antiviral protein, which binds to regions of high CpG to target for degradation.

CpG bias and G+C content. CpG sites or CG sites are regions of a polynucleotide where a cytosine nucleotide is followed by a guanine nucleotide in the linear sequence of bases along its 5′ 3′ direction. The frequency of CG in a given polynucleotide sequence is a function of the codon usage. By “CpG bias”, we mean the frequency of CpG dinucleotides in the gene. The frequency in one coding sequence may be 3%, but in a different sequence it may be 1%. The difference in frequency is 2%. Deviations of expected dinucleotide frequency is informed by the CG composition and codon usage. In gammaherpesvirus the CpG suppression of observed compared to expected is between 1.5-3.0% (Honess et al., 1989). By CpG bias which is “substantially the same” as the CpG bias of the native coding region for the corresponding native full-length herpesvirus polypeptide, we mean that the difference in frequency of CpG between codons in the immunogen coding region and the native coding region is less than 3%, such as less than 1%, such as less than 0.5%, such as less than 0.1%. “G+C content” is the frequency of the bases G and C in a polynucleotide sequence, and is also a function of the codon usage. The frequency in one coding sequence may be 60%, but in a different sequence it may be 40%. The difference in frequency is 20%. For example HHV2 has G+C median composition of 70%, while HHV7 is 36%. In contrast, between two closely related alphaherpesvirus, HHV1 and HHV2, the median G+C composition is 67.5% and 70% respectively, or for two closely related gammaherpesvirus, HHV6A and HHV6B, the median G+C composition is 42.4% and 42.8%. By G+C content which is “substantially the same” as the G+C content of the native coding region for the corresponding native full-length herpesvirus polypeptide, we mean that the difference in frequency of G+C between codons in the immunogen coding region and the native coding region is less than 5%, such as less than 2%, such as less than 1%, such as less than 0.5%. CpG dinucleotides have long been observed to occur with a much lower frequency in the sequence of vertebrate genomes than would be expected due to random chance, a phenomenon known as CpG suppression. HSV1 and HSV2 genomic sequences are biased in composition for high G+C content and are not CpG suppressed (Honess et al., 1989; Szpara et al., 2014). Therefore, genes from these viruses in the native codon usage can naturally stimulate the TLR9 innate signalling mechanisms for natural boosting of immune responses rather than to add synthetic CpG oligonucleotides as adjuvant. In contrast, using sequences with CpG suppression such as in the gammaherpesvirus can evade recognition by the ZAP protein and subsequent targeting for degradation (Takata et al., 2017).

Non-Coding Functional Motifs and Sequences

Kozak sequence. In an embodiment, each of the plurality of immunogen coding regions comprises a Kozak sequence which is capable of permitting initiation of translation of the herpesvirus polypeptide in the vertebrate cell with an efficiency which is substantially the same as the efficiency with which the Kozak sequence of the native coding region for the corresponding native full-length herpesvirus polypeptide permits initiation of translation in the vertebrate cell, such as wherein the Kozak sequence of each of the plurality of immunogen coding regions is identical to the Kozak sequence of the native coding region for the corresponding native full-length herpesvirus polypeptide. The Kozak sequence is a nucleic acid motif that functions as the protein translation initiation site in most eukaryotic mRNA transcripts. The sequence was originally defined as 5′-(gcc)gccRccAUGG-3 (IUPAC nucleobase notation summarized here) where the underlined nucleotides indicate the translation start codon, coding for Methionine; upper-case letters indicate highly conserved bases, i.e. the ‘AUGG’ sequence is constant or rarely, if ever, changes; ‘R’ indicates that a purine (adenine or guanine) is always observed at this position; a lower-case letter denotes the most common base at a position where the base can nevertheless vary; and the sequence in parentheses (gcc) is of uncertain significance. See Kozak M (1987) “An analysis of 5′-noncoding sequences from 699 vertebrate messenger RNAs”. Nucleic Acids Res. 15 (20): 8125-8148. doi:10.1093/nar/15.20.8125. For example, the Kozak sequence of the coding region of gH of HSV2 strain HG52 is ACGACCATGG (start codon underlined). Variation within the Kozak sequence alters the “strength” thereof. Kozak sequence strength refers to the favorability of initiation, affecting how much protein is synthesized from a given mRNA (Kozak, 2005). The strength of the Kozak sequence (Kozak, 2005) can be measured in a vertebrate cell by means known in the art, and is typically measured in a cell of the species for which the pharmaceutical composition is intended, typically a mammalian species, typically a human (Hernandez et al., 2019). Suitably, each of the immunogen coding regions comprises the native Kozak sequence so initiation of translation can ensue as during native infection of a cell.

3′ untranslated region. In an embodiment, each of the immunogen coding regions is operatively linked to a 3′ untranslated region (UTR) which permits substantially the same degree of mRNA stability of the immunogen coding region or transcript thereof, such as by virtue of comprising identical 3′ polyadenylation sequences. The 3′ UTR is found immediately following the translation stop codon and plays a critical role in translation termination as well as post-transcriptional modification. Polyadenylation is the addition of a poly(A) tail to a messenger RNA. The poly(A) tail consists of multiple adenosine monophosphates, such as 10 to 300 adenosine monophosphates. In eukaryotes, polyadenylation is part of the process that produces mature messenger RNA (mRNA) for translation. The process of polyadenylation begins as the transcription of a gene terminates. The 3′-most segment of the newly made pre-mRNA is first cleaved off by a set of proteins; these proteins then synthesize the poly(A) tail at the RNA's 3′ end. The poly(A) tail is important for the nuclear export, translation, and stability of mRNA. The tail is shortened over time, and, when it is short enough, the mRNA is enzymatically degraded. The 3′ polyadenylation sequence comprises a highly conserved AAUAAA sequence at 12-30 nt upstream of the cleavage site, and a U or GU rich sequence up to 30 nt downstream. Suitable 3′ polyadenylation sequences are from the SV40 virus, as provided in a standard expression vector, in a preferred embodiment derived from pCDNA3.1. All the gene insertions in the plasmid expression vector only include the 3′ stop codon, which is then followed by the standard 3′ untranslated region of the plasmid expression vector as stated above. In the Examples, all herpesvirus polypeptide genes as expressed in the plasmid expression vector contained the same 3′ polyadenylation sequences, in this case as in the standard SV40 expression vector, as described for pcDNA3 derived vectors (Invitrogen, Thermofisher), including pCMV6 series (Origene)(Andersson et al., 1989). While in the virus genes, differences in the 3′ sequence can affect RNA stability and turnover (Glaunsinger and Ganem, 2006), here the four genes are co-ordinately expressed and stable.

Promoters. In an embodiment in which the one or more nucleic acid molecules are deoxyribonucleic acid (DNA) polynucleotides, each of the immunogen coding regions is operatively linked to a 5′ promoter. Suitably, each coding region operatively linked to a 5′ promoter is capable of simultaneous gene expression in the vertebrate cell, such as by virtue of each coding region being linked to an identical 5′ promoter. A promoter is a sequence of DNA to which proteins bind that initiate transcription of mRNA from the DNA downstream of it. Suitably, a high expression promoter sequence is used. Suitably, the expression is constitutive in the vertebrate cell. In a mammalian cell, useful promoters may be obtained from Cytomegalovirus (CMV) and CAG hybrid promoter (hybrid of CMV early enhancer element and chicken beta-actin promoter) or Simian vacuolating virus 40 (SV40). A particularly suitable high expression promoter is from the immediate early gene of human cytomegalovirus (as in U.S. Pat. No. 5,385,839A) (Thomsen et al., 1984) as utilised in preferred embodiment of standard gene expression plasmid vector pCDNA3.1 or pCMV6 and related derivatives. In the Examples, plasmids were designed with promoter as used standardly in gene therapy applications, such that all of the gene products could be expressed simultaneously, unlike in the virus infected cells where the fusion regulator, gH/gL, is tightly controlled for expression only after DNA replication as a ‘late’ gene, while gD and gB are expressed early after infection.

Control sequences. The nucleic acid molecules may comprise one or more further control sequences as appropriate. The term “control sequences” means all nucleic acid sequences necessary for the expression of a polynucleotide encoding a herpesvirus polypeptide of the invention. Typically, for a DNA molecule, the control sequences include a promoter, and transcriptional and translational stop signals. Typically, at least one control sequence is foreign (i.e. from a different gene) to the immunogen coding region; thus the polynucleotide sequence is typically non-native. The control sequence may be a suitable transcription terminator sequence, which is recognized by a host cell to terminate transcription, such as the bovine growth hormone terminator. The terminator sequence may be operably linked to the 3′ terminus of the polynucleotide encoding the immunogen. Any terminator that is functional in the vertebrate cell may be used. Preferably, each of the immunogen coding regions is operatively linked to an identical transcriptional terminator. The control sequence may also be a suitable leader sequence, a nontranslated region of an mRNA that is important for translation by the vertebrate cell. The leader sequence may operably linked to the 5′ terminus of the immunogen coding region. Any leader sequence that is functional in the vertebrate cell may be used. The term “operably linked” means a configuration in which a control sequence is placed at an appropriate position relative to the coding sequence of a polynucleotide such that the control sequence directs the expression of the coding sequence.

Vectors and Expression Plasmids

The term “expression vector” means a linear or circular DNA molecule that comprises a polynucleotide encoding a herpesvirus polypeptide and is operably linked to control sequences that provide for its expression. Suitable DNA plasmid expression vectors are such as the standard plasmid eukaryotic gene expression vector pCDNA3.1 or derivatives (Accession No. LT727011.1) or pCMV6 (Accession no. AF239250) and related derivatives. Viral vectors are described in Draper S J, Heeney J L. Viruses as vaccine vectors for infectious diseases and cancer. Nat Rev Microbiol. 2010 January;8(1):62-73. doi: 10.1038/nrmicro2240. Although single DNA vaccination has been demonstrated for HSV2, multiple DNA vaccination has previously been attempted with some truncated genes, and was assumed to lower overall immune responses due to lowering the amounts of individual plasmid DNA components. A linear relationship was assumed between amount of DNA applied and amount of protein and thereby immune response attained. The approach of the present invention is different, to maximise the quality of the immune response by recreating the fusogenic complex in VLMs, which will form over time, a kinetic transition proceeding from engagement of the cellular receptor to triggering the fusion process via the fusion regulator and progressing from pre-fusion to post-fusion conformations of the membrane embedded fusogen. The inventor reasoned that it was likely that the multiple DNA plasmids would be transported into cells together in vivo, as demonstrated by the in vitro cellular fusion assays (Turner et al., 1998) and also in variable mixtures to form virus like membranes (VLMs) in different stages from pre-fusion to post-fusion conformations of the glycoprotein complexes as well as transition states. This could include gB SNPs as cited and summarised below in TABLE 4, that can stabilise pre-fusion or fusion conformations. Thus multiple expression plasmids may be used in vivo DNA vaccination, such as one encoding each herpesvirus polypeptide of the fusogenic complex. Alternatively, expression plasmids, which express more than one herpesvirus polypeptide, may be used with regulatory signals to allow independent expression.

A suitable RNA polynucleotide may comprise a 5′ terminal cap upstream of the immunogen coding region, such as 7mG(5′)ppp(5′)NlmpNp and/or may comprise one or more modified bases. Suitable modified bases are selected from pseudouridine, NI-methylpseudouridine, NI-ethylpseudouridine, 2-thiouridine, 4′-thiouridine, 5-methylcytosine, 2-thio-1-methyl-1-deaza-pseudou rid ine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Methods of manipulating and synthesising polynucleotides to generate suitable vectors and expression plasmids are known from Sambrook, J. and D. W. Russell, 2001 (Molecular Cloning: a laboratory manual, 3rd ed. Cold Spring Harbor Laboratory Press, Cold Spring Harbor, N.Y), and synthesized polynucleotides are available from commercial suppliers (for example Eurofinns).

Mutated gB and gD Polypeptides

In an embodiment, the gB polypeptide encoded by the immunogen coding region for gB comprises a mutation which stabilises gB polypeptide as a trimer in the fusion conformation, such as a mutation in the fusion associated domain I, such as a substitution at a position corresponding to amino acid position 262 of SEQ ID NO 9, such as wherein the substitution is a non-conservative substitution, such as small uncharged substitution, such as Isoleucine or Alanine.

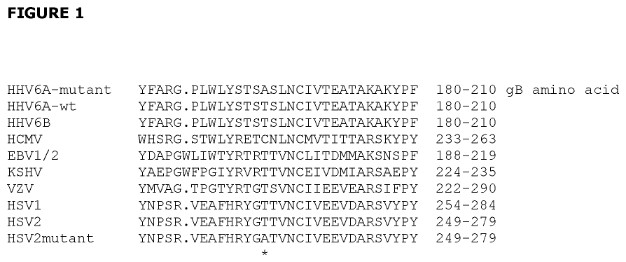

The inventor has identified mutations in gB which appear to stabilise the fusion conformation, and may aid the ability of the fusogenic complex to mediate cell fusion in order to increase cellular infection in culture. In the inventor's previous experimentation in human herpesvirus HHV-6A (Tweedy et al., 2017), deep sequencing using next generation sequencing was used to compare the original virus isolate sequence with passaged virus genomes. The results showed that gB was a site of one of the few coding changes, and the substitution Thr262Ala was identified. This was in the fusion associated domain I, a region modelled to increase stability of the gB trimer in the post-fusion conformation as demonstrated in structural stability changes affecting the free energy value of the conformation as shown (Tweedy et al., 2017). This contributed to the laboratory tissue culture adaptations for increased virus spread as demonstrated by dominance of the virus population over time. In this virus the cytopathic effect was overt cellular fusion rather than the controlled cell fusion event in HSV1 and HSV2. As described in the Examples, the inventor predicted the corresponding mutation in HSV2 gB using sequence alignment in FIG. 1 and introduced the coding change while maintaining the codon bias for the virus as noted above. The HSV2 mutated gB coding sequence (SEQ ID NO. 17) has the following features: Kozak sequence followed by ATG start codon at positions 1 to 3; A to G substitution at position 784 (corresponding codon change ACG to GCG, encoding Thr to Ala substitution). The HSV2 mutated gB amino acid sequence (SEQ ID NO. 8) has the following features: Domain I from amino acids Ala150 to Val358, including the amino acid substitution of Thr262Ala.

As described in Tweedy et al., 2017, there are crystal structures available for homologous gB molecules from HCMV, HSV, and EBV in the post-fusion conformation. Recent results also describe prefusion conformations for VZV or HSV1 gB (Oliver et al 2020, Vollmer et al 2020). Structural alignments of gB polypeptide trimers against any of these trimeric reference structures may be obtained by molecular modelling. Several tools and resources are available for retrieving and generating structural alignments. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747). Free energy predictions of the gB subunit-subunit interaction stability may be performed using web server mCSM, which predicts stability changes of a wide range of mutations from graph-based signatures encoding distance patterns between atoms (Pires D. E. V., Ascher D. B., Blundell T. L. mCSM: Predicting the effects of mutations in proteins using graph-based signatures. Bioinformatics. 2014; 30:335-342. doi: 10.1093/bioinformatics/btt691). In this way, putative mutations may be modelled to identify those that stabilise gB polypeptide as a trimer in the post-fusion conformation. If a mutation stabilises gB polypeptide as a trimer in the post-fusion conformation, the predicted free energy of the subunit-subunit interaction is lower than the free energy of the subunit-subunit interaction of a gB polypeptide trimer in the post-fusion conformation which is identical with the exception of the mutation.

By “mutation” we include substitution, insertion or deletion of one or more amino acids, or a combination of substitution, insertion and deletion. Substitutions are preferred. By virtue of the redundancy of the genetic code, a given substitution may be encoded by more than one possible codon. For all substitutions or other mutations, the invention encompasses all encoding nucleic acid molecules that encode the corresponding polypeptide mutation. Typically, suitable mutations may be identified in the fusion associated domain I of gB, as this domain may affect the trimer interface. In HSV2, domain I of gB is located at positions in the N-terminal domain region of Ala-150 to Thr-358 (SEQUENCE ID 9). The skilled person can identify domain I in herpesviruses by amino acid sequence alignment with HSV2, for example, by performing multiple alignments with other related human herpesvirus sequences and then modelling on know determined tertiary conformations in public domain databases such as Uniprot and using publically available standard tools to map to these (Burke and Heldwein, 2015). Examples of multiple alignments software is such as by using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. Other suitable software includes MUSCLE ((Multiple sequence comparison by log-expectation, Robert C. Edgar, Version 3.6, http://www.drive5.com/muscle; Edgar (2004) Nucleic Acids Research 32(5), 1792-97 and Edgar (2004) BMC Bioinformatics, 5(1):113) which may be used with the default settings as described in the User Guide (Version 3.6, September 2005).

With reference to variants of gB, positions are defined in relation to the full-length native HSV2 polypeptide sequence of strain HG52 (SEQUENCE ID NO. 9). However, the skilled person understands that the invention also relates to variants of other HHV gB polypeptides. For clarity, for gB other than the HSV2 gB of SEQ ID NO. 9, equivalent residues are favoured for mutation. Equivalent positions can be identified by comparing amino acid sequences using pairwise (e.g. ClustalW) or multiple (e.g. MUSCLE) alignments. Equivalent positions to Thr262 of gB of HSV2 are shown in FIG. 1.

In describing the gB or gD variants of the present invention, the nomenclature described below is adapted for ease of reference. The accepted IUPAC single letter or three letter amino acid abbreviation is employed. The term ‘point mutation’ and/or ‘alteration’ includes deletions, insertions and substitutions. For an amino acid substitution, the following nomenclature is used: Original amino acid, position, substituted amino acid. Accordingly, the substitution of threonine at position 262 with alanine is designated as “Thr262Ala” or “T262A”. For an amino acid deletion, the following nomenclature is used: Original amino acid, position*. Accordingly, the deletion of threonine at position 262 is designated as “Thr262*” or “T262*”. An insertion may be to the N-side (‘upstream’, ‘X-1’) or C-side (‘downstream’, ‘X+1’) of the amino acid occupying a position (‘the named (or original) amino acid’, ‘X’). For an amino acid insertion to the C-side (‘downstream’, ‘X+1’) of the original amino acid (‘X’), the following nomenclature is used: Original amino acid, position, original amino acid, inserted amino acid. Accordingly, the insertion of alanine after threonine at position 262 is designated “Thr262ThrAla” or “T262TA”. In such cases the inserted amino acid residue(s) are numbered by the addition of lower-case letters to the position number of the amino acid residue preceding the inserted amino acid residue(s). For an amino acid insertion to the N-side (‘upstream’, ‘X-1’) of the original amino acid (X), the following nomenclature is used: Original amino acid, position, inserted amino acid, original amino acid. Accordingly, the insertion of alanine before threonine at position 262 is designated “Thr262AlaThr” or “T262AT”. In such cases the inserted amino acid residue(s) are numbered by the addition of lower-case letters with prime to the position number of the amino acid residue following the inserted amino acid residue(s). Variants comprising multiple alterations are separated by addition marks (“+”). Where different alterations can be introduced at a position, the different alterations are separated by a comma.

Suitable substitutions may be conservative of non-conservative. As used herein, the term “conservative” amino acid substitutions refers to substitutions made within the same group, and which typically do not substantially affect protein function, such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine). By “conservative substitutions” is intended combinations such as Gly, Ala; Val, Ile, Leu; Asp, Glu; Asn, Gln; Ser, Thr; Lys, Arg; and Phe, Tyr. Non-conservative substitutions are other than within the above groups.

Suitably, a mutation, which may be substitution, deletion or insertion, is at a position corresponding to position 262 of SEQ ID NO 9. Substitution at this position is preferred. Suitably, substitutions are non-conservative, such as with Isoleucine or Alanine. The amino acid sequences or accession numbers of representative gB polypeptides, together with amino acid sequences of suitable variant gB polypeptides comprising a mutation at a position corresponding to position 262 of SEQ ID NO 9 are shown in Table 4 below. The position of the mutation and other exemplary mutations are also defined.

The exemplary mutations shown are variant gD or gB nucleotide sequences comprising mutations corresponding to and designed herein from the initial mutations for a subset of herpesvirus glycoproteins as disclosed in Montgomery et al., 1996; Kadelka et al., 2018; S. et al., 2020; Burn Aschner et al., 2020; Oliver et al., 2020 and Vollmer et al. 2020 and described herein. These SNPS add features modifying ADDC presentation for gD by limiting HVEM interaction or stabilising the prefusion gB conformation of the fusion complex as described above. The inventor reasoned that the mutations in the VZV gB structure and disclosed as aligned with HSV1, HCMV and EBV could also be aligned and deduced for HSV2, HHV6A, HHV6B, HHV7 and HHV8 and identifying features such as the additional cysteine residue in the gB Domain I alignment for HCMV and the N-linked glycosylation sites within the gB Domain IV beta-30 fold of HHV-6A and HHV-6B which would be independent of the alignment (FIG. 4). The prefusion stabilising gB SNP demonstrated in Domain III at 516His to Pro for HSV1 as disclosed in (Vollmer et al 2020) and aligned with VZV gB were also deduced by the inventor for HSV2 gB through alignment analyses (FIG. 5) as well as in the reference strains gB genes for HSV1 and VZV and the gene SNPs designed as indicated in Table 4.

TABLE 4
gD and gB SNP variants from representative HHV species strains

| | | | | |
|---|---|---|---|---|
| | | | Variant | |
| | | Wild-type | gB or gD | Position of |
| | | gB or gD | nucleotide | mutation in |
| | | polypeptide | sequence | polypeptide and |
| HHV | Reference | sequence or | comprising | Exemplary |
| species | Strain | accession no. | mutation | mutations. |

gD

| | | | | |
|---|---|---|---|---|
| HSV1 | Strain17, | YP_009137141.1 | SEQ ID 63 | 52. Gln52Pro |
| | NC_001806.2 | | | SEQ ID 64 |
| HSV2 | HG52, | YP_009137218.1 | SEQ ID 65 | 52. Gln52Pro |
| | NC_001798 | | | SEQ ID 66 |

gB

| | | | | |
|---|---|---|---|---|
| HSV1 | Strain17, | YP_009137102.1 | SEQ ID 91 | 267. Thr267Ala |
| | NC_001806.2 | | | |
| | | | SEQ ID 67 | 584, 585. |
| | | | | Gln584Ala Asn585Ala |
| | | | SEQ ID 68 | 585. Asn585Ala |
| | | | SEQ ID 69 | 555, 558. |
| | | | | Tyr555Ala Gln558Ala |
| | | | SEQ ID70 | 558. |
| | | | | Gln558Ala |
| | | | SEQ ID 132 | 516. |
| | | | | His516Pro |
| HSV2 | HG52, | YP_009137179.1 | SEQ ID 3 | 262. Thr262Ala |
| | NC_001798 | | | |
| | | | SEQ ID 71 | 581, 582. |
| | | | | Gln581Ala, Asn582Ala |
| | | | SEQ ID 72 | 582. Asn582Ala |
| | | | SEQ ID 73 | 652, 655. |
| | | | | Tyr652Ala, Gln655Ala |
| | | | SEQ ID 74 | 655. Gln655Ala |
| | | | SEQ ID 133 | 513. |
| | | | | His513Pro |
| VZV | Dumas, | NP_040154.2 | SEQ ID 92 | 273. Thr273Ala |
| | NC_001348.1 | | | |
| | | | SEQ ID 75 | 596, 597. |
| | | | | Gln596Ala, Asn597Ala |
| | | | SEQ ID 76 | 597. Asn597Ala |
| | | | SEQ ID 77 | 667, 670. |
| | | | | Tyr667Ala, Glu670Ala |
| | | | SEQ ID 78 | 670. Glu670Ala |
| | | | SEQ ID 134 | 526. |
| | | | | His526Pro |
| EBV- | B958 & RAJI | YP_401713.1 | SEQ ID 93 | 202. Thr272Ala |
| type1 | combination, | | | |
| | NC_007605.1 | | | |
| | | | SEQ ID 79 | 611, 615. |
| | | | | Phe611Ala, Glu615Ala |
| | | | SEQ ID 80 | 615. Glu615Ala |
| EBV- | AG876, | YP_001129508.1 | SEQ ID 94 | 202, Thr272Ala |
| type2 | NC_009334.1 | | | |
| | | | SEQ ID 79 | 611, 615. |
| | | | | Phe611Ala, Glu615Ala |
| | | | SEQ ID 80 | 615. Glu615Ala |
| HCMV | Merlin, | YP_081514.1 | SEQ ID 95 | 246, Thr246Ala |
| | NC_006273.2 | | | |
| | | | SEQ ID 81 | 633, 638. |
| | | | | Phe633Ala, Asp638Ala |
| | | | SEQ ID 82 | 638. |
| | | | | Asp638Ala |
| HHV-6A | U1102, | NP_042932.1 | SEQ ID 96 | 193, Thr193Ala |
| | NC_001664.4 | | | |
| | | | SEQ ID 83 | 671, 678. |
| | | | | His671Ala, Glu678Ala |
| | | | SEQ ID 84 | 678. Glu678Ala |
| HHV-6B | Z29, | NP_050220.1 | SEQ ID 97 | 193, Thr193Ala |
| | NC_000898.1 | | | |
| | | | SEQ ID 85 | 670, 677. |
| | | | | His670Ala, Glu677Ala |
| | | | SEQ ID 86 | 677. Glu677Ala |
| HHV-7 | RK, | YP_073779.1 | SEQ ID 98 | 190. Thr190Ala |
| | NC_001716.2 | | | |
| | | | SEQ ID 87 | 665, 673. |
| | | | SEQ ID 88 | Tyr665Ala, Glu673Ala |
| KSHV | GK18, | YP_001129354.1 | SEQ ID 99 | 218, Thr218Ala |
| | | | SEQ ID 89 | 608, 613. |
| | | | | Tyr608Ala, Asn613Ala |
| | | | SEQ ID 90 | 613. Asn613Ala |

In an embodiment, the immunogen coding region encoding gB possesses at least 95% sequence identity, such as at least 97% sequence identity, at least 99% sequence identify, at least 99.5% sequence identify or 100% sequence identity to a coding region for a variant gB polypeptide which differs from the native coding region for the corresponding native full-length gB polypeptide only in the codon corresponding to position 262 of SEQ ID NO 9 in the encoded variant gB polypeptide.

In an embodiment, the gD, gH and gL encoded by the immunogen coding regions have the amino acids sequences of SEQ ID NOs 10, 6 and 7 respectively, and the gB encoded by the immunogen coding region has the amino acids sequence of SEQ ID NO 8 or 9; such as wherein the gD, gH and gL immunogen coding regions have the nucleotide sequences of SEQ ID NOs 5, 1 and 2 respectively, or SEQ ID NOs 18, 15 and 16 respectively; and the gB immunogen coding region has the nucleotide sequence of SEQ ID NO 3, 4 or 17. Here, the mutated gB sequences are SEQ ID NOs 3, 17 and 8; and the wild-type gB sequences are SEQ ID NOs 4 and 9. In a further embodiment other exemplary SNP mutations in gD or gB as indicated on Table 4 may be utilised in the vaccine composition.

Pharmaceutical Composition

In addition to the one or more nucleic acid molecules, the pharmaceutical composition may comprise further components. Typically, the herpesvirus antigens are provided solely by one or more nucleic acid molecules, which express the antigens in vivo, and the pharmaceutical composition itself does not comprise a herpesvirus polypeptide antigen. Suitably, the one or more nucleic acid molecules encodes an immunomodulator, wherein the one or more nucleic acid molecules are capable of expressing the immunomodulator when introduced into the vertebrate cell; and/or the composition comprises an immunomodulator. An immunomodulator is an agent which stimulate the immune response. Suitable immunomodulators or ‘molecular’ adjuvants may be chemokines or cytokines, including viral chemokines. For example, cytokines are useful as a result of their lymphocyte regulatory properties, such as interleukin-12 (IL-12), GM-CSF and IL-18. Suitable chemokines include human CCL5 (RANTES) as described in the Examples, CCL17 (TARC), CCL18 (PARC), CCL20 (MIP3 alpha) or CCL19 (MIP3 beta) or CCL2 (MCP-1), CCL22 (MDC) and CXCL13 (BLC) as disclosed in conjunction with DNA vaccines in U.S. Pat. No. 7,384,641 B2. Suitable virus chemokines could include vmipii of Kaposi sarcoma associated herpesvirus (Pawig et al 2015) or U83 encoded molecules as described in U.S. Pat. No. 9,850,286 B2 and U.S. Pat. No. 8,940,686 B2, including U83A and variants thereof of HHV6 such as HHV6A. Suitable humanised viral chemokines are iciU83A-N(SEQ ID NOs 135 and 136) also referred to as ‘VIT’, virokine immune therapeutic, as described in the Examples and in PCT/EP2021/058776, Virokine Therapeutics Ltd entitled ‘Novel immunomodulator’. VIT is humanised iciU83A-N, a novel cDNA from a new spliced transcript variant from parent genes integrated at the human telomere as described from archaic HHV-6A genome (Tweedy et al 2016). By ‘VIT’ we also include coding region variants having at least 90%, such as at least 95%, such as 96%, 97%, 98%, 99% or at least 99.5% sequence identity to the iciU83A-N coding sequence as provided in SEQ ID NO. 135, and whose functional domains are described in Example 6. The skilled person may design and select variants which retain the functional activities of the native VIT as described in Example 6, and which have comparable functional activities thereto. The composition may comprise the immunomodulator in the form of a polypeptide, or it may be encoded in by a nucleic acid molecule within a gene-expression vector, such that it will be expressed as a polypeptide in the vertebrate cell along with the herpesvirus polypeptides. In other words, it will have the appropriate transcriptional and translational control sequences to enable expression of the polypeptide. By ‘VIT’ polypeptide as immunomodulator we also include variants having at least 90%, such as at least 95%, such as 96%, 97%, 98% or at least 99% sequence identity to the iciU83A polypeptide as provided in SEQ ID NO. 136.

The composition may comprise an adjuvant, although it is envisaged that an adjuvant may not be necessary, or may be necessary only in a quantity that is lower than would be required if the herpesvirus polypeptides were provided by means other than in the form of the fusogenic complex, or that a less toxic adjuvant only may be required. Thus compositions, which lack an adjuvant are also envisaged, as are those which contain only a chemical adjuvant which is appropriate for human use, such as alum.

Adjuvants are any substance whose admixture into the composition increases or otherwise modifies the immune response to an antigen. Adjuvants can include but are not limited to AlK(SO4)2, AlNa(SO4)2, AlNH(SO4)4, silica, alum, Al(OH)3, Ca3(PO4)2, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyl-L-alanyl-D-isoglutaminyl-L-alanine-2-(1′2′-dipalmitoyl-s-n-glycero-3-hydroxphosphoryloxy)-ethyla mine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium tuberculosis, substances found in Corynebacterium parvum, Bordetella pertussis, and members of the genus Brucella, liposomes or other lipid emulsions, Titermax, ISCOMS, Quil A, ALUN (see U.S. Pat. Nos. 58,767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21.

Additional adjuvants or compounds that may be used to modify or stimulate the immune response include ligands for Toll-like receptors (TLRs). In mammals, TLRs are a family of receptors expressed on DCs that recognize and respond to molecular patterns associated with microbial pathogens. Several TLR ligands have been intensively investigated as vaccine adjuvants. Bacterial lipopolysaccharide (LPS) is the TLR4 ligand and its detoxified variant mono-phosphoryl lipid A (MPL) is an approved adjuvant for use in humans. TLR5 is expressed on monocytes and DCs and responds to flagellin whereas TLR9 recognizes bacterial DNA or other foreign DNA containing CpG motifs. Oligonucleotides (OLGs) containing CpG motifs are potent ligands for, and agonists of, TLR9 and have been intensively investigated for their adjuvant properties. The genes from HSV1 and HSV2 are naturally high in CpG motifs, which also serve as natural ligands for TLR9 to stimulate innate immune signalling, instead of adding exogenous CpG adjuvants.

It is believed that the formation of the fusogenic complex in vivo in the form of VLMs may stimulate the immune response such that an adjuvant is not necessary, or only in a lower quantity, or only a less toxic adjuvant is needed. Expression of the fusogenic complex in vesicles and heterologous virus expression systems in vitro have been tried experimentally and these are used to study fusogenic processes (Rogalin and Heldwein, 2016; Vollmer and Grunewald, 2020). However, these are performed in laboratory cell lines, used in vitro in tissue culture, which may not be composed of the lipid formulations in primary specialised cell types, such as epithelial cell targets, in vivo. Expression of the glycoprotein genes for the fusion complex machine in vivo would allow the natural lipid formulation intrinsic to the fusion process. The lipid presentation of the herpesvirus glycoproteins may affect not only the complex presentation, the glycoprotein complex may also affect the lipid recruitment, for example in lipid rafts or with increased cholesterol, or affect membrane curvature. These lipid associations can also affect the formation of immunogenic extracellular vesicles. HHV gB can associate with these extracellular vesicles (Grabowska et al., 2020). Moreover, in vivo the membrane fusion event can stimulate innate immunity as through cell damage sensing via the cGAS-STING, TLR7 or TLR9 innate sensing pathways (Holm et al., 2012). Moreover, immune response triggers for some T cell subsets require interaction with lipids, and this may be facilitated by membrane glycoproteins clustering together in lipid rafts (Adams et al., 2015; Birkinshaw et al., 2015). Therefore, expressing the fusion complex machine aims to make responses that prevent spread and pathology in the body, i.e. cell to cell spread, which could also be applicable to therapeutic vaccines.

Suitably, the one or more nucleic acid molecules are provided as supercoiled DNA. The delivery of DNA is dependent on the conformation, greater delivery is in circular DNA in a supercoiled conformation (Liu, 2019). Therefore, synthesis maximises this in preparations that are highly concentrated and enables gene delivery in lower volumes. Suitably, the nucleic acid molecules are aggregated with an aggregating agent into types of nanoparticles; this may be particularly useful where the composition comprises more than one nucleic acid molecule. Suitably the composition comprises bupivacaine or levimasole. These anaesthetics have previously been demonstrated to increase expression by single plasmid DNA injection in preclinical small animal models (Pachuk et al., 2000) (Jin et al., 2004). However, this had not been used with multiple DNA plasmid injections. The present Examples combine bupivacaine with multiple DNA plasmids. Since it may also have the effect to cluster the molecules this can also aid the simultaneous delivery and expression. Similar approaches may use lipid based nanoparticles to form aggregates which could also be used (Liu, 2019).

The pharmaceutical composition of the first aspect of the invention is sterile, and is typically provided in a sealed sterile container. By “sterile” we include the meaning that the nucleic acid molecules have been filtered through a sterile bacterium-retaining filter, such as a 0.2 μm filter, and/or the nucleic acid molecules have been precipitated in a sterilising solution, such as in 70% ethanol. Any additional components included are also sterile, such that the overall composition is sterile. The additional components, if present may be sterilised together with the nucleic acid molecules. The composition is formulated for delivery in vivo (i.e. as a pharmaceutical preparation) unlike in vitro preparations which require an excipient such as DEAE dextran to cross a membrane barrier, by damaging it. In vivo this could have toxicity and rather the uptake of DNA are typically via intramuscular injection using sterile needles or via inhalers if via intranasal routes, for example, into tissue using physiological saline solutions to maximise uptake through native in vivo routes such as phagocytic mechanisms by specialised cells present in the tissue or conditioning of muscle cells via bupivacaine.

In an alternative aspect, the sterile pharmaceutical composition is not limited to being provided in a sealed sterile conditioner. All other features relevant to the pharmaceutical composition of the first aspect may also be applied to this aspect, including features of its uses in medicine.

The one or more nucleic acid molecules may be naked, that is, unassociated with any proteins, adjuvants or other agents, which affect the recipients' immune system. In this case, it is desirable for the polynucleotide(s) to be in a physiologically acceptable solution, such as, but not limited to, sterile saline or sterile buffered saline at physiological pH. Alternatively, the polynucleotides may be associated with polymers or liposomes, such as lecithin liposomes or other liposomes or other polymers known in the art, as a polynucleotide-liposome mixture, or the polynucleotide may be associated with an adjuvant known in the art to boost immune responses, or a protein or other carrier. Proteins, if present, are isolated from components with which they may be associated in nature. Agents which assist in the cellular uptake of polynucleotides, such as, but not limited to, calcium ions, may also be used. These agents are generally referred to as pharmaceutically acceptable carriers. Techniques for coating microprojectiles coated with polynucleotide are known in the art and are also useful in connection with this invention. The polynucleotides may be in a pharmaceutically acceptable carrier or buffer solution. Pharmaceutically acceptable carriers or buffer solutions are known in the art and include those described in a variety of texts such as Remington's Pharmaceutical Sciences. The carrier may be preferably a liquid formulation, and is preferably a buffered, isotonic, aqueous solution. Suitably, the vaccine composition has a pH that is physiologic, or close to physiologic. Suitably it is of physiologic or close to physiologic osmolarity and salinity and/or is endotoxin free. It may contain sodium chloride and/or sodium acetate. Pharmaceutically acceptable carriers may also include excipients, such as diluents, and the like, and additives, such as stabilizing agents, preservatives, solubilizing agents, and the like. As used herein, the term “pharmaceutically acceptable” means approved by a regulatory agency of US or EU or other government or listed in the U.S. Pharmacopeia or other generally recognized pharmacopeia for use in humans.

The pharmaceutical composition of the first aspect of the invention is provided in a sealed sterile container, such as in lyophilised, liquid or nebulised form. Suitable sealed sterile containers include a sealed sterile container for injection by needle, ampoule, phial, vial bottle, inhalers or the like. Suitably, the sealed sterile contained may be used in the delivery of the pharmaceutical composition, e.g. as a filled syringe and needle, inhaler etc. Suitably, the sealed sterile container may contain a single dose of the pharmaceutical composition.

Infectious Agent Antigens

In an embodiment, the one or more nucleic acid molecules encode one or more infectious agent antigens, wherein the one or more nucleic acid molecules are capable of expressing the one or more infectious agent antigens when introduced into the vertebrate cell; and/or wherein the pharmaceutical composition further comprises one or more infectious agent antigens. Suitable control sequences to enable expression of the one or more infectious agent antigens are as described above in relation to control sequences suitable for expression of herpesvirus polypeptides. For simplicity, it is preferred to provide one or more nucleic acid molecules encoding the one or more infectious agent antigens, and thus polypeptide antigens are preferred.

An “infectious agent antigen” is a molecule derived from an infectious agent, such as by virtue of being encoded in the genome of the infectious agent, that binds specifically to an antibody, or a T cell receptor (TCR) in conjunction with a major histocompatibility complex (MHC) molecule. Antigens that bind to antibodies include all classes of molecules, and are called B cell antigens. Suitable types of molecules include peptides, polypeptides, glycoproteins, polysaccharides, gangliosides, lipids, phospholipids, DNA, RNA, fragments thereof, portions thereof and combinations thereof. TCRs bind only peptide fragments of proteins complexed with MHC molecules; both the peptide ligand and the native protein from which it is derived are called T cell antigens. “Epitope” refers to an antigenic determinant of a B cell or T cell antigen. Where a B cell epitope is a peptide or polypeptide, it typically comprises three or more amino acids, generally at least 5 and more usually at least 8 to 10 amino acids. The amino acids may be adjacent amino acid residues in the primary structure of the polypeptide, or may become spatially juxtaposed in the folded protein. T cell epitopes may bind to MHC Class I or MHC Class II molecules. Typically, MHC Class I-binding T cell epitopes are 8 to 11 amino acids long. Class II molecules bind peptides that may be 10 to 30 residues long or longer, the optimal length being 12 to 16 residues. Peptides that bind to a particular allelic form of an MHC molecule contain amino acid residues that allow complementary interactions between the peptide and the allelic MHC molecule. The ability of a putative T cell epitope to bind to an MHC molecule can be predicted and confirmed experimentally.

Suitably, the infectious agent antigen comprises a B cell epitope and/or a T cell epitope and suitably comprises a peptide, polypeptide, carbohydrate, lipid, DNA or RNA. Suitable infectious agent antigens may be derived from viruses, bacteria, protozoans, prions, parasites, helminths, nematodes, or any other potential pathogen. Since the virus like membranes fusion complex would be expressed on the cell surface, preferred embodiments would be membrane surface expressed proteins as co-expressed antigens from pathogens. However, this is not exclusive since other antigens may be presented on MHC class I or II. Examples of viral antigens include coronavirus antigens, such as one or more antigens from SARS-Cov-2 coronavirus; human immunodeficiency virus (HIV) antigens such as products of the gag, pol, and env genes, the Nef protein, reverse transcriptase, and other HIV components; hepatitis, e.g., hepatitis A, B, and C, hepatitis viral antigens such as the S, M, and L proteins of hepatitis, the pre-S antigen of hepatitis B virus, hepatitis C viral RNA; influenza viral antigens hemagglutinin and neuraminidase and other influenza viral antigens; measles viral antigens such as SAG-1 or p30; rubella viral antigens such as proteins E1 and E2 and other rubella virus components; rotaviral antigens such as VP7sc components and other rotaviral components; respiratory syncytial viral antigens, such as the RSV fusion protein, the M2 protein; or one or more human papilloma virus antigens such as L1 proteins.

Examples of bacterial antigens include pertussis bacterial antigens such as pertussis toxin; diptheria bacterial antigens such as diptheria toxin or toxoid erythematosis; tetanus bacterial antigens such as tetanus toxin or toxoid; streptococcal bacterial antigens such as M proteins and other streptococcal bacterial antigen components. Fungal antigens which can be used include, but are not limited to Candida fungal antigen components; histoplasma fungal antigens, coccidiodes fungal antigens such as spherule antigens.

The presence of the herpesvirus fusogenic complex may cause the infectious agent antigen or antigens to be delivered to the immune system in such a way as to increase its/their immunogenicity. Thus, even natural antigens, which are not naturally very immunogenic can be used as the infectious agent antigen. Further, increased immunogenicity may allow dose sparing compared to currently licensed vaccines.

Where the one or more nucleic acid molecules encode the one or more infectious agent antigens, the one or more infectious agent antigens may be co-expressed with the herpesvirus polypeptides.

Methods of Manufacture

A corresponding aspect of the invention provides a method of making the pharmaceutical composition of the first aspect, comprising formulating the one or more nucleic acid molecules as defined in relation to the first aspect with one or more physiologically acceptable diluents or excipients as a sterile composition. The formulation may also comprise one or more further components such as adjuvants and/or immunomodulators as discussed in relation to the composition of the first aspect. The method may further comprise dispensing the pharmaceutical composition into a sterile container and sealing the sterile container, thereby providing a sterile sealed container.

In Vitro VLMs and Methods of Manufacture

A second aspect of the invention provides a pharmaceutical composition comprising a plurality of herpesvirus polypeptides in association with a lipid membrane, wherein the pharmaceutical composition is formed by expressing the plurality of herpesvirus polypeptides in vitro in human cells from one or more nucleic acid molecules comprising a plurality of immunogen coding regions which collectively encode the plurality of herpesvirus polypeptides, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus.

Suitably, the plurality of herpesvirus polypeptides in association with a lipid membrane are provided in the form of membrane vesicles or whole cells. Human cells are used to prepare the VLMs for human use, to avoid the presence of unwanted non-human antigens in the preparation. Suitable human cells include cells derived from a human patient who is to be administered the membrane vesicles or cells.

Suitably, the one or more nucleic acid molecules are as defined in relation to the first aspect of the invention. Suitably, the pharmaceutical composition further comprises one or more infectious agent antigens. Features of the infectious agent antigens are as described in relation to the first aspect of the invention. Excipients for delivery of cells expressing the genes would typically be at physiological pH and salinity in cell buffered systems in sterile solutions for delivery by standard infusions in excipients as used for CAR-T cells. Typically, the composition is sterile excepting the living cells. The pharmaceutical composition may be provided in a sterile sealed container such as a drip bag for intravenous infusion to a patient.

A corresponding aspect of the invention provides a method of making the pharmaceutical composition of the second aspect, comprising introducing the one or more nucleic acid molecules as defined according to the second aspect into human cells in vitro, allowing the human cells to express the plurality of herpesvirus polypeptides from the one or more nucleic acid molecules, thereby obtaining the plurality of herpesvirus polypeptides in association with a lipid membrane. The method may further comprise collecting membrane vesicles or whole cells comprising the plurality of herpesvirus polypeptides in association with a lipid membrane, and optionally purifying the membrane vesicles or whole cells. The method may further comprise formulating the pharmaceutical composition with one or more infectious agent antigens, and/or an immunomodulator, and/or an adjuvant. Features relating to the pharmaceutical composition of the second aspect are also suitable in relation to the method of preparation. Suitable methods of introducing the one or more nucleic acid molecules into the cells include transfection, as known in the art. Where whole cells are to be used, white blood cells could be collected as for preparation of CAR-T cells and the one or more nucleic acid molecules introduced instead of CAR-T genes. Methods for producing CAR-T cells are described in Dotti et al., 2014, and may be adapted accordingly. Suitable methods for preparing membrane vesicles, such as exosomes, comprising herpesvirus polypeptides are as described in Zeev-Ben-Mordehai et al., 2014. The membrane vesicles may be secreted by transfected cells into the culture medium, and purified by methods such as differential centrifugation, as described supra. A further method of preparing membrane vesicles by engineering cells to express membrane proteins and culturing cells is described in WO 2015/011478.

Medical Uses

In another aspect, the invention provides the pharmaceutical composition of either the first or second aspect for use in medicine. The pharmaceutical composition of the first aspect of the invention is typically intended for use in animals, typically mammals, typically humans. Although not exclusively, the pharmaceutical composition of the second aspect is primarily intended for use in human subjects.

Herpesvirus Infection

Suitably, the pharmaceutical composition of either the first or second aspect is provided for use in a method of inducing an immune response to a herpesvirus; or for use in a method of preventing or treating a herpesvirus infection. In the alternative is provided a method of inducing an immune response to a herpesvirus, comprising administering an effective amount of the pharmaceutical composition of either the first or second aspect to a subject; or a method of preventing or treating a herpesvirus infection, comprising administering an effective amount of the pharmaceutical composition of either the first or second aspect to a subject.

By “inducing an immune response”, we include any humoral and/or cellular immune response. Suitably, the immune response comprises an antibody response to one or more of the herpesvirus polypeptides. The antibody titer produced by pharmaceutical compositions, also referred to herein as vaccines, of the invention may be a neutralizing antibody titer. Antibody titer is determined or measured by enzyme-linked immunosorbent assay (ELISA); and/or by microneutralization assay for example as described (Atanasiu et al., 2018; Cairns et al., 2014; Cairns et al., 2006; Gompels et al., 1991; Bourne et al., 2003). A neutralization titer is typically expressed as the highest serum dilution required to achieve a 50% reduction in the number of virus plaque forming unit, pfu. In some embodiments, an effective amount of a vaccine results in incremental increases, such as 2-fold to 200-fold (e.g., 20- or 200-fold), in serum neutralizing antibodies against HHV, relative to an unimmunised control. For example, the increase in neutralizing antibody titre compared to an unimmunised control may be between 10-fold and 200-fold, such as about 50-fold, about 100-fold or about 200-fold.

The efficacy of the vaccine in inducing an immune response to the antigen can be determined using animal experiments, such as in preclinical studies including protection from pathogen challenge. For example, a mouse or guinea pig can be immunized with a vaccine. After the appropriate period of time to allow immunity to develop against the antigen, for example two weeks, a blood sample is tested to determine the level of antibodies, termed the antibody titre, using ELISA. In some instances, the animal is immunized and, after the appropriate period of time, challenged with the herpesvirus to determine if protective immunity against the herpesvirus has been achieved. Suitable animal tests may be used to develop an appropriate combination of herpesvirus encoding nucleic acid molecules and other vaccine components, such as adjuvant. Testing in humans can be contemplated after efficacy is demonstrated in animal models. Any known methods for immunization, including formulation of a vaccine composition and selection of doses, route of administration and the schedule of administration (e.g. primary and one or more booster doses) can be used (e.g. see Vaccines: From concept to clinic, Paoletti and McInnes, eds, CRC Press, 1999).

Suitable uses of the vaccines are for prevention and/or treatment of HHV in humans and other mammals. Pharmaceutical compositions, also referred to herein as vaccines, can be used as therapeutic or prophylactic agents. Vaccines of the present disclosure may be used to provide prophylactic protection from HHV. It is possible, although less desirable, to administer the vaccine to an infected individual to achieve a therapeutic response. Dosing may need to be adjusted accordingly. Vaccines can be administered once, twice, three times, four times or more, but it is likely sufficient to administer the vaccine once (optionally followed by a single booster). Vaccines may be administered to children (aged under 18 years) or adults (aged over 18 years).

Prevention or treatment of HHV may be assessed in animal models. Typically, the animal will be vaccinated, and then, after a period of time to allow the immune response to develop, will be challenged with live virus. The Examples report successful vaccination of guinea pigs with HSV2 polynucleotides and protection from primary genital disease and viral burden following vaginal challenge. Positive effects on reducing recurrent disease, and virus shedding including asymptomatic shedding were observed, together with a significant reduction in latent viral burden. Accordingly, prevention of HHV infection according to the invention, such as infection with HSV2 or HSV1, may include protection from acute disease and/or infection; protection from establishing latent infection; protection from reactivating latent infection and/or viral transmission; and/or protection from latent viral recurrence and disease. Treatment of HHV infection, such as infection with HSV2 or HSV1, may also reduce or protect from establishing latent infection, reactivating latent infection and/or viral transmission, and/or latent viral recurrence and disease. Reactivation of latent infection means production of more virus, and may be relevant for viral transmission even if disease recurrence e.g. a lesion is not observed. For disease phases occurring after the acute phase of infection, the Examples show a particularly beneficial effect of including a nucleic acid molecule encoding an immunomodulator in the vaccine composition. An immunomodulator can affect immune cell recruitment affecting control of latency by cellular immunity. Accordingly, it is preferred for the one or more nucleic acid molecules of the composition to encode an immunomodulator, wherein the one or more nucleic acid molecules are capable of expressing the immunomodulator when introduced into the vertebrate cell; and/or for the composition to comprise an immunomodulator. Suitable immunomodulators include chemokines such as CCL5 or VIT, or other cytokines, as described herein.

Other animal models for testing of vaccines for other herpesviruses are known in the art and include murine, guinea pig and non-human primate models for disease, for example neuropathology and genital infections of alphaherpesvirus (HHV1, HHV2, HHV3), immune pathology in betaherpesvirus (HHV5, HHV6A/B, HHV7) and lymphoproliferations in gammaherpesvirus (HHV4 and HHV8) (Belshe et al., 2014; Bernstein et al., 2019; Bernstein et al., 1999; Dogra and Sparer, 2014; Fujiwara and Nakamura, 2020; Kollias et al., 2015; Zerboni et al., 2014) Typically, the prevention or treatment of HHV is with respect to the same species of HHV from which the herpesvirus polypeptides are derived. However, as there is some similarity between herpesvirus polypeptides of different species or types, cross-protection may be achieved. For example, an HSV2 vaccine may protect against HSV1 infection, and vice versa.

The actual dosage amount of a composition of the present invention administered to an animal or human patient, i.e. the effective amount, can be determined by physical and physiological factors such as body weight, severity of condition, the type of disease being treated, previous or concurrent therapeutic interventions, idiopathy of the patient and the route of administration. The practitioner responsible for administration will, in any event, determine the concentration of active ingredient(s) in a composition and appropriate dose(s) for the individual subject. In some embodiments the dosage of the polynucleotide or VLM is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 g, 40-100 μg, 100-400 μg, 200-400 μg, or 300-400 μg per dose.

The vaccine may be administered to the subject by intramuscular, intradermal, subcutaneous, intravaginal or intranasal administration, such as by intradermal or intramuscular injection. Embodiments include intravaginal topical application or intranasal inhalation using an inhaler or via intranasal application of drops of solution containing the vaccine formulation. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Other Infections

Where the pharmaceutical composition of either the first or second aspect comprises an infectious agent antigen, or the one or more nucleic acid molecules of the pharmaceutical composition of the first aspect encode one or more infectious agent antigens, the pharmaceutical composition may be provided for use in a method of inducing an immune response to the one or more infectious agent antigens; or for use in a method of preventing or treating an infection caused by an infectious agent which comprises the one or more infectious agent antigens. In the alternative is provided a method of inducing an immune response to one or more infectious agent antigens, comprising administering (i) an effective amount of a pharmaceutical composition of either the first or second aspect comprising one or more infectious agent antigens, or (ii) an effective amount of a pharmaceutical composition of the first aspect wherein the one or more nucleic acid molecules encode one or more infectious agent antigens, to a subject. In the alternative is provided a method of preventing or treating an infection caused by an infectious agent which comprises one or more infectious agent antigens, comprising administering (i) an effective amount of a pharmaceutical composition of either the first or second aspect comprising one or more infectious agent antigens, or (ii) an effective amount of a pharmaceutical composition of the first aspect wherein the one or more nucleic acid molecules encode one or more infectious agent antigens, to a subject.

Considerations with respect to identifying induction of an immune response, assessing prevention or treatment of an infection, identifying a suitable dose and route of administration are as described above in respect of herpesvirus vaccination. Similar procedures can be followed with other infections targeted via additional antigen targets with respective preclinical animal models followed by standard clinical trials as known in the art.

### REFERENCES

Bernstein D I, Earwood J D, Bravo F J, Cohen G H, Eisenberg R J, Clark J R, Fairman Cardin R D. Vaccine. 2011 Mar. 3; 29(11):2071-8. doi: 10.1016/j.vaccine.2011.01.005. Epub 2011 Jan. 14. PMID: 21238569. Effects of herpes simplex virus type 2 glycoprotein vaccines and CLDC adjuvant on genital herpes infection in the guinea pig.
Tweedy J, Spyrou M A, Pearson M, Lassner D, Kuhl U, Gompels U A. Viruses. 2016 Jan. 15; 8(1):19. doi: 10.3390/v8010019. PMID: 26784220. Complete Genome Sequence of Germline Chromosomally Integrated Human Herpesvirus 6A and Analyses Integration Sites Define a New Human Endogenous Virus with Potential to Reactivate as an Emerging Infection.
Andersson, S., Davis, D. L., Dahlback, H., Jornvall, H., and Russell, D. W. (1989). Cloning, structure, and expression of the mitochondrial cytochrome P-450 sterol 26-hydroxylase, a bile acid biosynthetic enzyme. J Biol Chem 264, 8222-8229.
Atanasiu, D., Saw, W. T., Lazear, E., Whitbeck, J. C., Cairns, T. M., Lou, H., Eisenberg, R. J., and Cohen, G. H. (2018). Using Antibodies and Mutants To Localize the Presumptive gH/gL Binding Site on Herpes Simplex Virus gD. J Virol 92.
Athey, J., Alexaki, A., Osipova, E., Rostovtsev, A., Santana-Quintero, L. V., Katneni, U., Simonyan, V., and Kimchi-Sarfaty, C. (2017). A new and updated resource for codon usage tables. BMC Bioinformatics 18, 391.
Beilstein, F., Cohen, G. H., Eisenberg, R. J., Nicolas, V., Esclatine, A., and Pasdeloup, D. (2019). Dynamic organization of Herpesvirus glycoproteins on the viral envelope revealed by super-resolution microscopy. PLoS Pathog 15, e1008209.
Belshe, R. B., Heineman, T. C., Bernstein, D. I., Bellamy, A. R., Ewell, M., van der Most, R., and Deal, C. D. (2014). Correlate of immune protection against HSV-1 genital disease in vaccinated women. J Infect Dis 209, 828-836.
Belshe, R. B., Leone, P. A., Bernstein, D. I., Wald, A., Levin, M. J., Stapleton, J. T., Gorfinkel, I., Morrow, R. L., Ewell, M. G., Stokes-Riner, A., et al. (2012). Efficacy results of a trial of a herpes simplex vaccine. N Engl J Med 366, 34-43.
Bender, F. C., Whitbeck, J. C., Ponce de Leon, M., Lou, H., Eisenberg, R. J., and Cohen, G. H. (2003). Specific association of glycoprotein B with lipid rafts during herpes simplex virus entry. J Virol 77, 9542-9552.
Bernstein, D. I. (2020). Use of the guinea pig model of genital herpes to evaluate vaccines and antivirals: review. Antiviral Res, 104821.
Bernstein, D. I., Flechtner, J. B., McNeil, L. K., Heineman, T., Oliphant, T., Tasker, S., Wald, A., Hetherington, S., and Genocea study, g. (2019). Therapeutic HSV-2 vaccine decreases recurrent virus shedding and recurrent genital herpes disease. Vaccine 37, 3443-3450.
Bernstein, D. I., Tepe, E. R., Mester, J. C., Arnold, R. L., Stanberry, L. R., and Higgins, T. (1999). Effects of DNA immunization formulated with bupivacaine in murine and guinea pig models of genital herpes simplex virus infection. Vaccine 17, 1964-1969.
Bourne N, Bravo F J, Francotte M, Bernstein D I, Myers M G, Slaoui M, Stanberry L R. Herpes simplex virus (HSV) type 2 glycoprotein D subunit vaccines and protection against genital HSV-1 or HSV-2 disease in guinea pigs. J Infect Dis. 2003 Feb. 15; 187(4):542-9. doi: 10.1086/374002. Burke, H. G., and Heldwein, E. E. (2015). Crystal Structure of the Human Cytomegalovirus Glycoprotein B. PLoS Pathog 11, e1005227.
Burn Aschner, C., Loh, L. N., Galen, B., Delwel, I., Jangra, R. K., Garforth, S. J., Chandran, K., Almo, S., Jacobs, W. R., Ware, C. F., and Herold, B. C. (2020). HVEM signaling promotes protective antibody-dependent cellular cytotoxicity (ADCC) vaccine responses to herpes simplex viruses. Science Immunology 5, eaax2454.
Cairns, T. M., Ditto, N. T., Atanasiu, D., Lou, H., Brooks, B. D., Saw, W. T., Eisenberg, R. J., and Cohen, G. H. (2019). Surface Plasmon Resonance Reveals Direct Binding of Herpes Simplex Virus Glycoproteins gH/gL to gD and Locates a gH/gL Binding Site on gD. J Virol 93.
Cairns, T. M., Fontana, J., Huang, Z. Y., Whitbeck, J. C., Atanasiu, D., Rao, S., Shelly, S. S., Lou, H., Ponce de Leon, M., Steven, A. C., et al. (2014). Mechanism of neutralization of herpes simplex virus by antibodies directed at the fusion domain of glycoprotein B. J Virol 88, 2677-2689.
Cairns, T. M., Shaner, M. S., Zuo, Y., Ponce-de-Leon, M., Baribaud, I., Eisenberg, R. J., Cohen, G. H., and Whitbeck, J. C. (2006). Epitope mapping of herpes simplex virus type 2 gH/gL defines distinct antigenic sites, including some associated with biological function. J Virol 80, 2596-2608.
Catusse J, Parry C M, Dewin D R, Gompels U A. Inhibition of HIV-1 infection by viral chemokine U83A via high-affinity CCR5 interactions that block human chemokine-induced leukocyte chemotaxis and receptor internalization. Blood. 2007 May 1;109(9):3633-9. doi: 10.1182/blood-2006-08-042622. Epub 2007 Jan. 5. PMID: 17209056
Catusse J, Clark D J, Gompels U A. 2009. CCR5 signalling, but not DARC or D6 regulatory, chemokine receptors are targeted by herpesvirus U83A chemokine which delays receptor internalisation via diversion to a caveolin-linked pathway. J Inflamm. 2009 Jul. 30; 6:22. doi: 10.1186/1476-9255-6-22. PMID: 19643012
Clark D J, Catusse J, Stacey A, Borrow P, Gompels U A. 2013. Activation of CCR2+ human proinflammatory monocytes by human herpesvirus-6B chemokine N-terminal peptide. J Gen Virol. 2013 July;94(Pt 7):1624-1635. doi: 10.1099/vir.0.050153-0. PMID: 23535574
Davison, A. J., Eberle, R., Ehlers, B., Hayward, G. S., McGeoch, D. J., Minson, A. C., Pellett, P. E., Roizman, B., Studdert, M. J., and Thiry, E. (2009). The order Herpesvirales. Arch Virol 154, 171-177.
Dewin D R, Catusse J, Gompels U A. Identification and characterization of U83A viral chemokine, a broad and potent beta-chemokine agonist for human CCRs with unique selectivity and inhibition by spliced isoform. J Immunol. 2006 Jan. 1; 176(1):544-56. doi: 10.4049/jimmunol.176.1.544. PMID: 16365449
Dogra, P., and Sparer, T. E. (2014). What we have learned from animal models of HCMV. Methods Mol Biot 1119, 267-288.
Dotti, G., Gottschalk, S., Savoldo, B., and Brenner, M. K. (2014). Design and development of therapies using chimeric antigen receptor-expressing T cells. Immunol Rev 257, 107-126.
Eisenberg, R. J., Atanasiu, D., Cairns, T. M., Gallagher, J. R., Krummenacher, C., and Cohen, G. H. (2012). Herpes virus fusion and entry: a story with many characters. Viruses 4, 800-832.
Fujiwara, S., and Nakamura, H. (2020). Animal Models for Gammaherpesvirus Infections: Recent Development in the Analysis of Virus-Induced Pathogenesis. Pathogens 9.
Gary, E. N., and Weiner, D. B. (2020). DNA vaccines: prime time is now. Curr Opin Immunol 65, 21-27.
Glaunsinger, B. A., and Ganem, D. E. (2006). Messenger RNA turnover and its regulation in herpesviral infection. Adv Virus Res 66, 337-394.
Gompels, U. A., Carss, A. L., Saxby, C., Hancock, D. C., Forrester, A., and Minson, A. C. (1991). Characterization and sequence analyses of antibody-selected antigenic variants of herpes simplex virus show a conformationally complex epitope on glycoprotein H. J Virol 65, 2393-2401.
Gompels, U. A., and Minson, A. C. (1989). Antigenic properties and cellular localization of herpes simplex virus glycoprotein H synthesized in a mammalian cell expression system. J Virol 63, 4744-4755.
Grabowska, K., Wachalska, M., Graul, M., Rychlowski, M., Bienkowska-Szewczyk, K., and Lipinska, A. D. (2020). Alphaherpesvirus gB Homologs Are Targeted to Extracellular Vesicles, but They Differentially Affect MHC Class II Molecules. Viruses 12.
Grunwald, T., and Ulbert, S. (2015). Improvement of DNA vaccination by adjuvants and sophisticated delivery devices: vaccine-platforms for the battle against infectious diseases. Clin Exp Vaccine Res 4, 1-10.
Hamby, S. E., and Hirst, J. D. (2008). Prediction of glycosylation sites using random forests. BMC Bioinformatics 9, 500.
Hernandez, G., Osnaya, V. G., and Perez-Martinez, X. (2019). Conservation and Variability of the AUG Initiation Codon Context in Eukaryotes. Trends Biochem Sci 44, 1009-1021.
Hilterbrand, A. T., and Heldwein, E. E. (2019). Go go gadget glycoprotein!: HSV-1 draws on its sizeable glycoprotein tool kit to customize its diverse entry routes. PLoS Pathog 15, e1007660.
Holm, C. K., Jensen, S. B., Jakobsen, M. R., Cheshenko, N., Horan, K. A., Moeller, H. B., Gonzalez-Dosal, R., Rasmussen, S. B., Christensen, M. H., Yarovinsky, T. O., et al. (2012). Virus-cell fusion as a trigger of innate immunity dependent on the adaptor STING. Nat Immunol 13, 737-743.
Honess, R. W., Gompels, U. A., Barrell, B. G., Craxton, M., Cameron, K. R., Staden, R., Chang, Y. N., and Hayward, G. S. (1989). Deviations from expected frequencies of CpG dinucleotides in herpesvirus DNAs may be diagnostic of differences in the states of their latent genomes. J Gen Virol 70 (Pt 4), 837-855.
Itzhaki R F. Overwhelming Evidence for a Major Role for Herpes Simplex Virus Type 1 (HSV1) in Alzheimer's Disease (AD); Underwhelming Evidence against. Vaccines (Basel). 2021 Jun. 21; 9(6):679. doi: 10.3390/vaccines9060679.
Jin, H., Li, Y., Ma, Z., Zhang, F., Xie, Q., Gu, D., and Wang, B. (2004). Effect of chemical adjuvants on DNA vaccination. Vaccine 22, 2925-2935.
Kadelka, C., Liechti, T., Ebner, H., Schanz, M., Rusert, P., Friedrich, N., Stiegeler, E., Braun, D. L., Huber, M., Scherrer, A. U., et al. (2018). Distinct IgG1-driven antibody response landscapes demarcate individuals with broadly HIV-1 neutralising activity. J. Exp. Med. 215, 1589-1608.
Knipe, D. M., and Howley, P. M., eds. (2013). Fields Virology, 6th edn (Wolters Kluwer).
Kollias, C. M., Huneke, R. B., Wigdahl, B., and Jennings, S. R. (2015). Animal models of herpes simplex virus immunity and pathogenesis. J Neurovirol 21, 8-23.
Kowalzik F, Schreiner D, Jensen C, Teschner D, Gehring S, Zepp F. mRNA-Based Vaccines. Vaccines (Basel). 2021 Apr. 15; 9(4):390. doi: 10.3390/vaccines9040390.
Kozak, M. (2005). Regulation of translation via mRNA structure in prokaryotes and eukaryotes. Gene 361, 13-37.
Lamb Y N. BNT162b2 mRNA COVID-19 Vaccine: First Approval. Drugs. 2021 March;81(4):495-501. doi: 10.1007/s40265-021-01480-7.
Lange, P. T., Lagunoff, M., and Tarakanova, V. L. (2019). Chewing the Fat: The Conserved Ability of DNA Viruses to Hijack Cellular Lipid Metabolism. Viruses 11.
Liu, M. A. (2019). A Comparison of Plasmid DNA and mRNA as Vaccine Technologies. Vaccines (Basel) 7.
Looker, K. J., Johnston, C., Welton, N. J., James, C., Vickerman, P., Turner, K. M. E., Boily, M. C., and Gottlieb, S. L. (2020a). The global and regional burden of genital ulcer disease due to herpes simplex virus: a natural history modelling study. BMJ Glob Health 5, e001875.
Looker, K. J., Magaret, A. S., May, M. T., Turner, K. M. E., Vickerman, P., Newman, L. M., and Gottlieb, S. L. (2017). First estimates of the global and regional incidence of neonatal herpes infection. Lancet Glob Health 5, e300-e309.
Looker, K. J., Welton, N. J., Sabin, K. M., Dalai, S., Vickerman, P., Turner, K. M. E., Boily, M. C., and Gottlieb, S. L. (2020b). Global and regional estimates of the contribution of herpes simplex virus type 2 infection to HIV incidence: a population attributable fraction analysis using published epidemiological data. Lancet Infect Dis 20, 240-249.
Milne, R. S., Mattick, C., Nicholson, L., Devaraj, P., Alcami, A., and Gompels, U. A. (2000). RANTES binding and down-regulation by a novel human herpesvirus-6 beta chemokine receptor. J Immunol 164, 2396-2404.
Minaya, M. A., Jensen, T. L., Goll, J. B., Korom, M., Datla, S. H., Belshe, R. B., and Morrison, L. A. (2017). Molecular Evolution of Herpes Simplex Virus 2 Complete Genomes: Comparison between Primary and Recurrent Infections. J Virol 91.
Montgomery, R. I., Warner, M. S., Lum, B. J., and Spear, P. G. (1996). Herpes simples virus-1 entry into cells mediated by a novel member of the TNF/NGF receptor family. Cell 87, 427-436.
Muggeridge, M. I. (2000). Characterization of cell-cell fusion mediated by herpes simplex virus 2 glycoproteins gB, gD, gH and gL in transfected cells. J Gen Virol 81, 2017-2027.
Muthumani, K., Wise, M. C., Broderick, K. E., Hutnick, N., Goodman, J., Flingai, S., Yan, J., Bian, C. B., Mendoza, J., Tingey, C., et al. (2013). HIV-1 Env DNA vaccine plus protein boost delivered by EP expands B- and T-cell responses and neutralizing phenotype in vivo. PLoS One 8, e84234.
Nugent, T., and Jones, D. T. (2009). Transmembrane protein topology prediction using support vector machines. BMC Bioinformatics 10.
Oliver, S. L., Xing, Y., Chen, D. H., Roh, S. H., Pintilie, G. D., Bushnell, D. A., Sommer, M. H., Yang, E., Carfi, A., Chiu, W., and M., A. A. (2020). A glycoprotein B-neutralizing antibody structure at 2.8 Å uncovers a critical domain for herpesvirus fusion initiation. Nat Commun. 11, 4141.
Pachuk, C. J., Ciccarelli, R. B., Samuel, M., Bayer, M. E., Troutman, R. D., Zurawski, D. V., Schauer, Higgins, T. J., Weiner, D. B., Sosnoski, D. M., et al. (2000). Characterization of a new class of DNA delivery complexes formed by the local anesthetic bupivacaine. Biochim Biophys Acta 1468, 20-30.
Patrone, M., Coroadinha, A. S., Teixeira, A. P., and Alves, P. M. (2016). Palmitoylation Strengthens Cholesterol-dependent Multimerization and Fusion Activity of Human Cytomegalovirus Glycoprotein B (gB). J Biol Chem 291, 4711-4722.
Lukas Pawig, Christina Klasen, Christian Weber, Jurgen Bernhagen, Heidi Noels Diversity and Inter-Connections in the CXCR4 Chemokine Receptor/Ligand Family: Molecular Perspectives Front Immunol. 2015 Aug. 21; 6:429. doi: 10.3389/fimmu.2015.00429. eCollection 2015. PMID: 26347749 PMCID: PMC4543903 DOI: 10.3389/fimmu.2015.00429
Rogalin, H. B., and Heldwein, E. E. (2016). Characterization of Vesicular Stomatitis Virus Pseudotypes Bearing Essential Entry Glycoproteins gB, gD, gH, and gL of Herpes Simplex Virus 1. J Virol 90, 10321-10328.
Rosa S S, Prazeres D M F, Azevedo A M, Marques M P C. mRNA vaccines manufacturing: Challenges and bottlenecks. Vaccine. 2021 Apr. 15; 39(16):2190-2200. doi: 10.1016/j.vaccine.2021.03.038.
S., B., Gupta, A., and Ravetch, J. V. (2020).
The role of IgG Fc receptors in antibody-dependent enhancement. Nat Rev Immunol, 11.
Stanberry, L. R., Spruance, S. L., Cunningham, A. L., Bernstein, D. I., Mindel, A., Sacks, S., Tyring, S., Aoki, F. Y., Slaoui, M., Denis, M., et al. (2002). Glycoprotein-D-adjuvant vaccine to prevent genital herpes. N Engl J Med 347, 1652-1661.
Strasser, J. E., Arnold, R. L., Pachuk, C., Higgins, T. J., and Bernstein, D. I. (2000). Herpes simplex virus DNA vaccine efficacy: effect of glycoprotein D plasmid constructs. J Infect Dis 182, 1304-1310.
Szpara, M. L., Gatherer, D., Ochoa, A., Greenbaum, B., Dolan, A., Bowden, R. J., Enquist, L. W., Legendre, M., and Davison, A. J. (2014). Evolution and diversity in human herpes simplex virus genomes. J Virol 88, 1209-1227.
Takata, M. A., Goncalves-Carneiro, D., Zang, T. M., Soil, S. J., York, A., Blanco-Melo, D., and Bieniasz, P. D. (2017). CG dinucleotide suppression enables antiviral defence targeting non-self RNA. Nature 550, 124-127.
Thomsen, D. R., Stenberg, R. M., Goins, W. F., and Stinski, M. F. (1984). Promoter-regulatory region of the major immediate early gene of human cytomegalovirus. Proc Natl Acad Sci USA 81, 659-663.
Trujillo, J. A., Fleming, E. L., and Perlman, S. (2013). Transgenic CCL2 expression in the central nervous system results in a dysregulated immune response and enhanced lethality after coronavirus infection. J Virol 87, 2376-2389.
Turner, A., Bruun, B., Minson, T., and Browne, H. (1998). Glycoproteins gB, gD, and gHgL of herpes simplex virus type 1 are necessary and sufficient to mediate membrane fusion in a Cos cell transfection system. J Virol 72, 873-875.
Tweedy J, Spyrou M A, Pearson M, Lassner D, Kuhl U, Gompels U A. Complete Genome Sequence of Germline Chromosomally Integrated Human Herpesvirus 6A and Analyses Integration Sites Define a New Human Endogenous Virus with Potential to Reactivate as an Emerging Infection. Viruses. 2016 Jan. 15; 8(1):19. doi: 10.3390/v8010019.
Tweedy, J. G., Escriva, E., Topf, M., and Gompels, U. A. (2017). Analyses of Tissue Culture Adaptation of Human Herpesvirus-6A by Whole Genome Deep Sequencing Redefines the Reference Sequence and Identifies Virus Entry Complex Changes. Viruses 10.
Verbeke R, Lentacker I, De Smedt S C, Dewitte H. dawn of mRNA vaccines: The COVID-19 case. J Control Release. 2021 Mar. 30; 333:511-520. doi: 10.1016/j.jconre1.2021.03.043
Vollmer, B., and Grunewald, K. (2020). Herpesvirus membrane fusion—a team effort. Curr Opin Struct Biol 62, 112-120.
Vollmer, B., Prazak, V., Vasishtan, D., Jefferys, E. E., Hernandez-Duran, A., Vallbracht, M., Klupp, B. G., Mettenleiter, T. C., Backovic, M., Rey, F. A., Topf, M. and Grunewald, K. (2020) The prefusion structure of herpes simplex virus glycoprotein B. Science Advances 6, eabc1726.
White, E. M., Stampfer, S. D., and Heldwein, E. E. (2020). Expression, Purification, and Crystallization of HSV-1 Glycoproteins for Structure Determination. Methods Mol Biol 2060, 377-393.
Youssef, S., Maor, G., Wildbaum, G., Grabie, N., Gour-Lavie, A., and Karin, N. (2000). C—C chemokine-encoding DNA vaccines enhance breakdown of tolerance to their gene products and treat ongoing adjuvant arthritis. J Clin Invest 106, 361-371.
Zeev-Ben-Mordehai, T., Vasishtan, D., Siebert, C. A., Whittle, C., and Grunewald, K. (2014). Extracellular vesicles: a platform for the structure determination of membrane proteins by Cryo-EM. Structure 22, 1687-1692.
Zerboni, L., Sen, N., Oliver, S. L., and Arvin, A. M. (2014). Molecular mechanisms of varicella zoster virus pathogenesis. Nat Rev Microbiol 12, 197-210.

The invention is further described by the following examples that should not be construed as limiting the scope of the invention.

### Example 1: In Silico Design and In Vitro Expression of HSV2 Fusogenic Complex

Prior to testing in an in vivo model system, genes for the ‘virus-like membranes’ were designed in silico and characterised in vitro. Although the genes necessary and sufficient for HSV in vitro cellular fusion had been characterised (Muggeridge, 2000; Turner et al., 1998), they had not been tested together for this in vivo. Instead, the antigenic properties of the individual genes or encoded proteins or mixtures of proteins were characterised, here the focus was on producing large amounts of protein either increasing protein production as encoded from genes, or to produce recombinant proteins exogenously. To increase protein production from genes, the focus for DNA immunisation was on use of delivery devices, such as electroporation or nanoparticles, or to optimise the expression by modifying to human codon usage using standard codon usage tables (Liu, 2019). The focus on cell fusion was to characterise the mechanism of cell fusion, which required crystallisation studies of large amounts of protein produced in artificial vesicular systems or from recombinant virus proteins or using exogenous extracellular vesicles produced by overexpression of gB (Rogalin and Heldwein, 2016; Vollmer and Grunewald, 2020; White et al., 2020; Zeev-Ben-Mordehai et al., 2014). Introducing genes with modified codon usage from pathogens can alter their subsequent folding and functional activities as reported. Previous studies were not focused on the function in cell fusion of the genes, but rather to optimise the production of protein for antigenic stimulus, primarily linear epitopes.

Instead, the present discovery combines these approaches on antigen versus fusion production and designs genes for delivery in combination that would mimic the natural fusion ‘motor’ from the virus as embedded in cellular membranes. The individual virus glycoproteins are known to associate with lipids, for example gB with lipid rafts (Bender et al., 2003; Lange et al., 2019), and the fusion components to gather together on the membrane in cascade interactions leading up to cell fusion (Beilstein et al., 2019; Cairns et al., 2019). Therefore, our invention is to express together, these 4 fusion complex genes in vivo, which would provide a ‘virus-like membrane’, VLM. This would enable exposure of transient forms of the fusion complex to elicit effective immunity. Further it could be triggered by the binding event, in this case native gD to its receptors. This binding could also be retargeted.

To have a natural, wild type, cell fusion ‘motor’ we synthesised the wild type genes all from the same strain. Critically, reports show combinations of glycoproteins from different strains can have different activities. The four genes targeted together were gD, gB, gH and gL. To determine and verify their wild type composition, genomic analyses was undertaken. A strain was selected that was representative in the human population as reported for HG52, which is also a reference strain (Minaya et al., 2017; Szpara et al., 2014). All available sequences were compared and analysed. This was compared to the updated release of the genome reference strain of HSV2, HG52 as determined by deep sequencing of stores of the original isolates stocks using Illumina based next generation sequencing (NCBI Reference Sequence: NC_001798.2). This revealed some mutations in previous reports of this strain's sequence and verified the current gene sequences for synthesis. Each gene ORF was linked to the native Kozak consensus sequence. In addition, HSV2 gB was modified to include a similar mutation to that described for HHV-6A gB (Tweedy et al., 2017)(FIG. 1).

The coding regions of the genes, including endogenous Kozak sequences are shown in SEQ ID NO. 15, 16, 17 and 18 encoding gH, gL, mutated gB, and gD respectively. These were synthesized and cloned into an expression plasmid pCMV6 in frame with a 5′ promoter for gene transcription from HCMV IE gene and 3′ polyadenylation site from SV40 virus for termination. Each gene retained native signals for translation including Kozak sites, initiating methionine codon and termination codons. Each gene retained the native G+C compositional bias and increased CpG bias, for triggering the TLR9 innate signalling pathway. The plasmid expression constructs were based on the standard pCDNA3.1 DNA plasmid expression vector, such as pCMV6neo which contains a 5′ promoter for gene expression provided by the Human cytomegalovirus immediate early, IE1, gene, with RNA transcription start site, kozak consensus sequence as described above, initiating methionine start codon, followed by terminating stop codon, site for polyadenylation from SV40 virus and flanked by restriction enzyme cloning sites. The plasmid also contains a neomycin resistance gene for selection of expression in vitro in cells. The plasmid expression constructs were validated by confirming the in-frame sequences using both Sanger and next generation sequencing. The plasmid sizes are as follows: 8338 bp (gH2), 6520 bp (gL2), 7027 bp (gD2) and 8560 bp (gB2).

Their expression was confirmed using in vitro transcription translation as well as transient expression in cell lines in vitro. Further, in vitro cellular fusion assays can be evaluated following methods to assay polykarocyte formation as described (Muggeridge, 2000; Turner et al., 1998). This was shown with transfection of the HSV VLM genes including the modified gB gene together with EGFP expression plasmid, such as expressed from standard plasmid expression vector, pCDNA3.1, and used as a fluorescent marker. Human HEK293 cells were transfected using a commercial transfection reagent (for example, Nanocin plasmid, Tecrea Ltd or Lipofectamine, Invitrogen) with the plasmid expression constructs then after 48 h post transfection examined under a fluorescent microscope. Cells receiving the EGFP expression plasmid fluoresced green, with light excitation peak at wavelength 488 nm and and emission peak at 509 nm. The VLM constructs with EGFP versus EGFP alone showed significantly increased cytomegalia showing cellular fusion as measured by cellular dimensions (p<0.001). Addition of the VIT construct to the VLM constructs were also similarly increased for cellular fusion compared to EGFP alone (p<0.01). Gene expression from all the plasmids was determined analysing the RNA from the transfected cells using standard known procedures for those skilled in the art. Briefly, oligonucleotide primers specific to the individual genes were designed or standard commercial primers from the expression plasmid vector sequence either side of the inserted genes. RNA was treated with DNAse (Invitrogen) to enzymatically remove any remaining transfected plasmid DNA, then using RT-PCR commercial kits (for example Superscript IV One-Step RT-PCR and Platinum Superfi RT, Invitrogen) expression examined with or without the reverse transcriptase, RT, present. All transfected cells showed specific expression as shown by amplification of the cDNA produced from the RNA only in presence of RT using the gene specific oligonucleotide primers, while all negative controls for the buffer used remained negative. RNA expression from the expression plasmids were determined from both individual gene transfections into the cells as well as with the VLM, or VLM+VIT combined gene expression plasmid DNA constructs. These same assays can be used to determine expression and effects on cell fusion by SNPs in the glycoprotein genes as in Table 4. For example, effects on the fusogenic complex of the gB pre-fusion or fusion stabilising mutations cited in Table 4 can be compared using the cell fusion assay.

### Example 2: In Vitro Expression of HSV2 Fusogenic Complex with Human Chemokine CCL5

To evaluate delivery and related expression with the VLMs with an exogenous gene, the VLM genes were co-expressed with a human immunomodulator gene. This could be any modulator of immune stimulation, and here we selected a human chemokine CCL5 which modulates attraction of antigen presenting and effector immune cells critical to establish effective immunity, and one of the downstream effectors from stimulators of innate immunity, for example via TLR pathways. This would serve to both enhance and focus the response. It also could serve as a marker for transgene expression. A similar strategy was followed as for the VLM genes. The native gene was determined by evaluating all versions of the gene sequence on NCBI. This was combined with its native Kozak sequence then the DNA synthesized, SEQ ID 11, and cloned into an expression plasmid as based on the standard DNA plasmid pCDNA3.1 as described above. The in-frame sequence was again determined by both Sanger and next generation sequencing. The plasmid size was 6121 bp. Expression can be verified by in vitro transcription translation analyses as well as within transfected cell lines using cDNA analyses and ELISA analyses of the protein produced using standard methods to detect CCL5 expression as described (Human CCL5/RANTES quantikine kit, R&D systems) (Milne et al., 2000).

### Example 3: Testing the HSV2 DNA Vaccines in a Preclinical In Vivo Model

To evaluate the efficacy of the delivered gene components of ‘virus like membranes’, a preclinical model of infectious disease was used to test its ability as a vaccine or immunotherapeutic to provide protection. This was tested as a prophylactic DNA vaccine formulation in an HSV2, herpes simplex virus type 2, model for sexually transmitted disease, STD. Recent estimates show this virus affects over 400 million people around the world. The lifelong recurrent STD, genital herpes, requires continual antiviral therapy using toxic drugs, and despite this available small molecule therapy, HSV is a significant contributor to HIV transmission, promoting over 30% cases from recent analyses (Looker et al., 2020b). A further serious consequence of the infection is neonatal disease with high mortality in the newborn, over 60% without treatment (Looker et al., 2017) a complication of the leading cause of GUD, genital ulcer disease, which recent estimates show affect 5% of the world population, approximately 200 million people (Looker et al., 2020a). Moreover, co-moribities have been suggested involving the inflammatory effects of HSV recurrences, for example in Alzheimer's disease and there is epidemiological evidence for a role with potential utility for treating patients with this effect (Itzhaki 2021). Therefore, there is major unmet medical need for a vaccine to prevent infection or disease, yet even though there have been clinical trials, none to date has been successfully produced. Developing prophylactic vaccines would protect discordant couples, neonatal disease, and those affected by HIV. Producing the ‘virus like membranes’ derived from HSV2 could generate specific immunity to protect against acute virus challenge.

There are preclinical small animal models used to evaluate protective efficacy of vaccines to prevent HSV2 disease. These have used both murine and guinea pig systems. The guinea pig model is preferred from a regulatory perspective since it models recurrence of the STD as occurs in people and also shows correlates of immune protection also mirrored in people (Bernstein, 2020; Strasser et al., 2000). Furthermore, efficacy can be compared to the current gold standard of the subunit protein vaccine used previously in clinical trials. This was composed of the excreted external domain of glycoprotein gD from HSV2 formulated with licensed vaccine adjuvants, alum and MPL (Belshe et al., 2012). The ‘Herpevac’ trials evaluated this vaccine in women to protect against HSV2, and the trial results showed some protection against HSV1 but not HSV2 (Belshe et al., 2014), complicated by the observation of changed epidemiology of genital herpes in USA with increasing roles for HSV1 over HSV2 in causing pathology. The correlate of protection was neutralising antibodies, so this acts as a good comparator to evaluate new vaccines. Indeed recent and retrospective analyses of this model show that this vaccine requires immunological boosting to be effective in people (Belshe et al., 2014; Bernstein, 2020; Stanberry et al., 2002). This showed that increasing responses could provide protection.

Using nucleic acid delivery has advantages over subunit protein vaccines. The subunit protein vaccine has disadvantages, since the recombinant produced glycoprotein needs to be purified and standardised for dosing, and it has issues of stability and toxicity to test. Nucleic acid vaccines, such as those deploying DNA, have superior advantages due to increased safety, ability to scale, no cold chain required, and low production costs. A significant drawback historically has been inability to produce sufficient protective immunity in clinical trials. Nonetheless, this delivery has potential, as DNA vaccines have been successfully deployed in aquaculture, using naked DNA plasmid infection to protect salmon from virus disease and licensed by the European Medicines Agency (Liu, 2019). Therefore, for use in people, DNA vaccines could be efficacious if they stimulate appropriate immune responses for protection.

We tested our ‘virus like membranes’ composed of the fusion machine genes from HSV2 as described above. Naked DNA plasmids composed of our 4 genes in plasmid preparations as described in Example 1 combined with bupivacaine were tested in the guinea pig model for HSV2. The ‘virus-like membrane’ DNA formulation comprised each of the four plasmids in an amount of 50 to 100 μg per 100 μL (referred to interchangeably as VLM or gD-VLM). The ‘virus-like membrane’ DNA with CCL5 had the same formulation together with 100 μg per 100 μL plasmid DNA expressing encoded human chemokine CCL5, SEQ ID11, as described in Example 2. Each DNA combination was formulated with Bupivacaine, as described (Bernstein et al., 1999; Pachuk et al., 2000). The formulations also included sterile excipients composed of physiological saline buffered at physiological pH 7. The negative control was no vaccine, and the positive control was the gold standard gD subunit vaccine used previously in the clinical trial as described. This is gD306, the external domain to amino acid 306, a secreted derivative formulated with MPL and alum, which has similarities in composition to AS04 as described in Belshe et al., 2012.

The prophylactic preclinical vaccine trial design used protocols as previously established (Bernstein et al., 1999; Strasser et al., 2000) using two or three immunisations. The guinea pig model used here had two immunisations injected intramuscularly, separated by intervals of three weeks, then challenged by virus delivered by the intravaginal route. The two dose immunisation schedule was a suboptimal protocol used in order to evaluate the extent of protection. This was from previous experience using solely gD expressing plasmid DNA in the standard Guinea Pig model (Bernstein, 2020; Strasser et al., 2000). This was also followed by a higher virus challenge titer than used previously, again to test efficacy. Twelve animals were in each group and either received no injection, negative control, or injections of the protein subunit vaccine positive control or the two test DNA vaccines by the intramuscular route in the hindquarter as described (Bernstein et al., 1999).

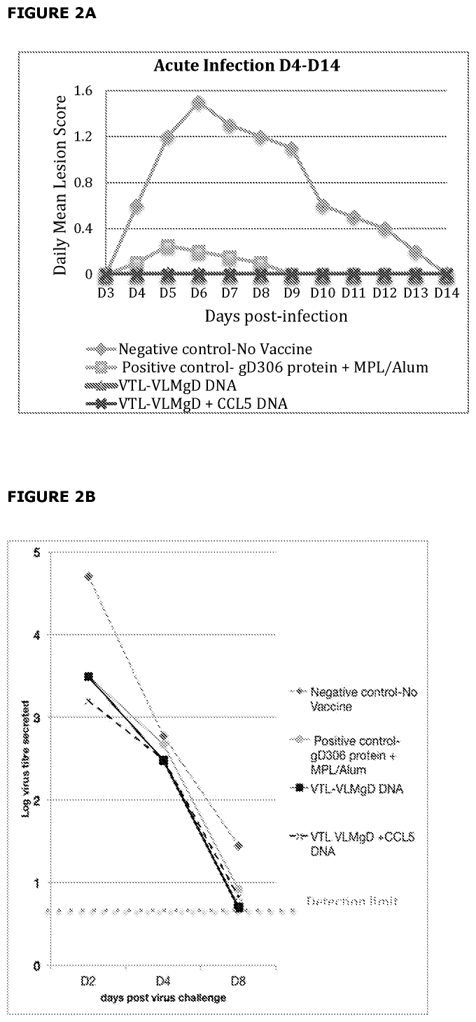

The trial proceeded and was followed by challenge virus inoculation and assay for protective efficacy of the immunisations. The two immunisations were given in a volume of 0.1 ml separated by three weeks. Then three weeks after the final immunisation, the virus challenge was administered to the vaginal vault, 1×10⁶ pfu, HSV-2 strain MS as described (Bernstein et al., 1999). After the initial immunisations, there were no adverse events recorded for any of the animals. Then after the virus challenge, all animals were examined daily from day 3 to day 14 for symptoms of acute disease, namely vaginal lesions, and for secreted virus titres in swabs according to the method of Bernstein et al., 1999 (FIG. 2A). The results showed that all the animals in the negative control group experienced the typical vesicular lesions of acute disease. In the positive control group using the gold standard gD protein subunit vaccine with adjuvant MPL/alum there were only 25% (3/12) animals which showed symptoms of disease. While in the ‘virus-like membrane’ DNA vaccination group, there were 0% (0/12) animals with any symptoms, showing there was complete protection. The groups with the combination of ‘virus-like membrane’ DNA together with CCL5 were similar showing complete protection with 0/12 animals with any symptoms. The positive control and two test DNA vaccines showed significant protection compared to the negative control (p<0.01), while the ‘virus-like membrane’ DNA vaccination was best in class.

Comparisons of the total lesions observed during acute infection, over the fourteen days period followed, also showed the remarkable efficacy of the immunisation with the ‘virus like membrane’, VLM, DNA vaccines compared to that of the negative control. These showed lesions completely eliminated in the VLM DNA vaccinated animals. The animals that had received no immunisations prior to virus challenge, the negative control group, had a mean total lesion severity score of 8.29 (SD 6.57) versus the positive control protein subunit vaccine of 0.67 (SD 1.48) with scoring matrix as described (Bernstein et al., 1999). In contrast, the VLM DNA vaccinated animals had no lesions over the total observation period, complete protection, while the VLM DNA plus human chemokine CCL5 vaccinated animals also showed complete protection, highly significant compared to the negative control (p<0.0001). This is in stark contrast to previous reports using only the gD plasmid DNA to immunise in the same guinea pig model. Here there was incomplete protection giving a total lesion score of 2.7(+/−0.7) compared to the negative control used there of 5.9(+/−0.5) (Bernstein et al., 1999; Strasser et al., 2000).

The data analysing virus secretion inhibition supported the above data assaying protection from pathology. As shown in FIG. 2B, by day 2 post challenge the gD-VLM showed significant protection reducing virus titres secreted as detected in vaginal swabs compared to animals with no vaccine (gD-VLM 3.49 log+/−0.4 compared to no vaccine 4.7 log+/−0.53, p=0.006) equivalent to efficacy of the subunit protein vaccine with adjuvant MPL/Alum, a similar formulation to that trialed in patients showing protection against HSV1 (protein subunit vaccine 3.49 log+/−0.91). The gD-VLM+CCL5 showed similar results (3.2 log+/−0.87, significantly reduced compared to no vaccine, p=0.003). By 8 days post challenge the protein subunit vaccine showed incomplete protection with 3/12 animals with mean virus still detectable at 0.92 logs+/−0.49. In contrast, all the gD-VLM DNA vaccine treated animals showed complete protection, 0/10 animals with detectable virus (0.7 log cut-off), compared to no vaccine with 5/9 animals with detectable virus at mean log titres of 1.45+/−0.99, p=0.02.

To summarise, the VLM DNA vaccines showed highly efficient protection from HSV2 challenge, from both pathology and virus secretion, which exceeded that demonstrated by the previous clinically trialed gD protein subunit vaccine. The VLM DNA vaccine showed complete protection to acute virus challenge and demonstrated the utility of this innovation. This supports progress to human trial evaluation.

### Example 4. Testing the HSV2 DNA Vaccines In Vivo in a Preclinical Model Shows Vaccine Utility in Preventing Disease and Virus Infection

In order to further evaluate the utility of the VLM—virus like membrane technology, further assays were conducted to evaluate its efficacy in preventing virus infection. The assays performed included scoring for disease severity as demonstrated in EXAMPLE 3 together with follow up for plaque titration of vaginal swabs to determine effects on virus infection post virus challenge after the immunisation protocol described in EXAMPLE 3. The efficacy endpoints were incidence and severity of acute disease plus the effect on virus vaginal replication as measured by virus titration by plaque assy. This EXAMPLE therefore represents further analyses of the experiment conducted in EXAMPLE 3.

Statistics were performed for all in vivo examples using Graphpad Prism with one-way Anova using Dunnett's test for multiple comparisons for the different vaccine treatments vs no vaccine. Where non-gaussian distributions, non-parametric comparisons using Wilcoxon test were used. Significance was noted at P values<0.05 (*), <0.01 (**), <0.001 (***).

4.1 Incidence and Severity of Acute Disease

The DNA formulations, gD-VLM (SEQ IDs 15, 16, 17, 18) are tested in comparisons to negative control-no vaccine- or positive control-gD protein (secreted gD306)+MPL/alum adjuvant—similar to the formulation used in earlier clinical trials showing partial protection to HSV2 and HSV1 in women, this benchmarks clinical utility (Belshe et al 2012).

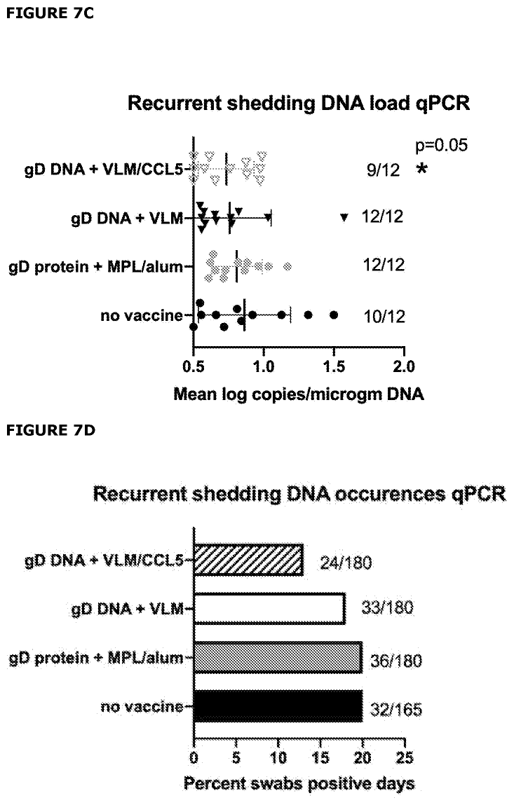

The gD-VLM DNA formulation alone and combined with CCL5 both show complete protection exceeding that shown for the positive control of gD protein MPL/alum. Daily mean lesion scores are shown in FIG. 2A. The further analyses are of the total acute mean lesion score for the individual animals in each test cohort (12 animals, 11 animals in the no vaccine group), as shown in FIG. 6A. The VLM vaccine treatments have significantly lowered scores, though differences between the vaccine treatments in this sample size do not reach significance.

4.2. Effect on Virus Vaginal Replication

The results on the effect of the treatment on lesion development are compared to effects on virus shedding during the primary disease. Analyses of the significantly lowered vaginal virus load correlates with the disease protection shown with close to log reduction of virus shed as for the gD protein formulation, using the immunisation schedule that is suboptimal for the positive control subunit gD protein.

By 8 days post virus challenge both the gD-VLM DNA vaccine formulations had significantly reduced virus shedding to undetectable levels in almost all animals, p<0.01 (FIG. 6B). Protocols for these titrations are as described (Bernstein et al 1999, 2020; Bernstein 2019).

Therefore, combining results from Examples 3 and 4, the gD-VLM DNA vaccine formulations show high efficacy in preventing HSV2 acute disease and infection.

### Example 5. Testing the HSV2 DNA Vaccines In Vivo in a Preclinical Model Shows Vaccine Utility in Preventing Detectable Latent Infection and as Therapeutic for Preventing Recurrent Persistent Infectious Disease

Here the VLM (SEQ ID 15, 16, 17, 18) containing vaccines are evaluated for utility in preventing recurrent disease and latent, persistent infection. The in vivo preclinical model described in EXAMPLE 3 has extended follow up to 63 days post-challenge with virus, HSV2, after the two dose immunisation schedule with the vaccines. The assays performed include DNA PCR of vaginal shedding swabs and DNA PCR of the sites for latent infection, the dorsal root ganglion, DRG, and spinal cord. The efficacy endpoints were the effects on recurrent disease, asymptomatic shedding and latent viral burden. The limit on detection were marked and measured for virus quantification at 0.7 log pfu/ml and for qPCR undetectable below the limit of detection at 0.5 log microgm copies DNA/ml. DNA PCR methods are as described (Bernstein et al., 1999).

5.1 Effect on Recurrent Disease

The effects of the vaccine treatments on recurrences of disease were analysed 15 to 63 days post infection challenge. Cumulative daily lesions were plotted and total mean lesion scores per individual compared.

This showed that the VLM DNA vaccines with CCL5 could prevent recurrent disease.

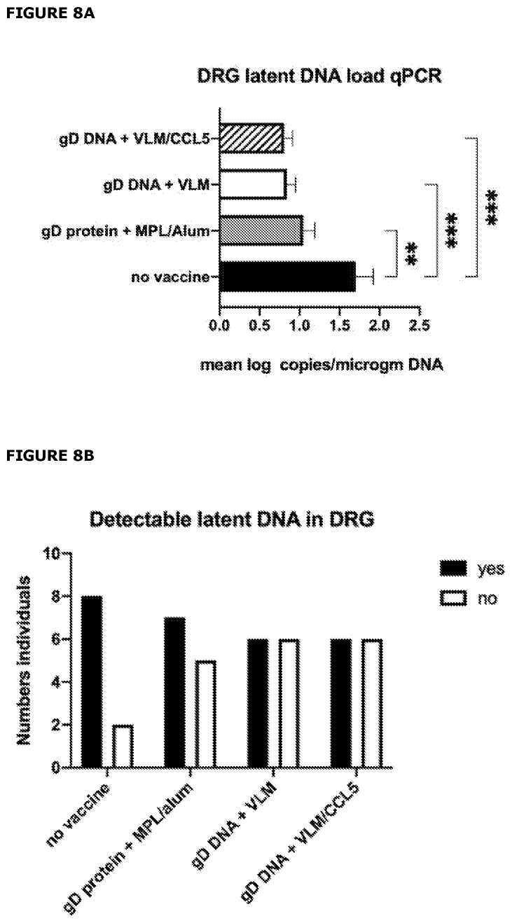

Distinctly, the chemokine DNA addition to the VLM treatment showed control of recurrent lesions. In comparison to the no vaccine treatment, protection from recurrent lesions was shown with the gD-VLM+CCL5 treatment, p=0.1, trend; and positive control gD subunit protein vaccine p<0.01 (FIGS. 7A and 7B). In the half of animals with disease recurrences, so measurable lesions per day as scored from 1-4 as described, from redness to ulcer (Bernstein et al 1999), the cumulative lesion days for the groups treated with VLM+CCL5 formulations and gD protein subunit vaccine formulation were reduced by a third to half.

While, half of the animals were completely protected from any disease recurrences with the gD-VLM/CCL5 formulation (6/12 50%), as shown in FIG. 7A.

5.2. Effect on Asymptomatic Virus Shedding

The effects of the vaccine treatments were tested for reductions on virus shedding after evidence for virus reactivation after day 20 post virus challenge. To do this the DNA load assayed in vaginal swabs by quantitative PCR was used as a surrogate for virus secretion. This detects both symptomatic shedding and asymptomatic shedding, i.e. virus secreted with no evidence for lesion pathology.

Analyses of recurrent shedding events and the total mean load in vaccinated compare to unvaccinated animals were done. While the gD protein subunit vaccine had no effect, the VLM+CCL5 vaccine significantly reduced virus shedding with almost half the overall load and a third less shedding events with a quarter of animal with no detectable shedding (FIGS. 7C and 7D).

5.3. Effect on Latent Viral Burden

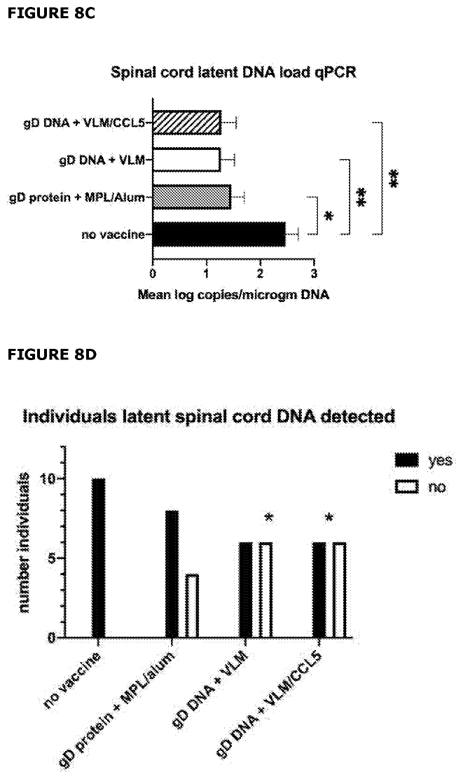

The effects on establishment of latency at sites in the dorsal root ganglia and the spinal cord were assayed. At the end of the study, day 63 post virus challenge, the DNA present was quantified using qPCR (Bernstein et al 199) at these sites of latency. Similar to the trend on virus secretion, analyses of the total mean DRG loads showed all vaccines significantly reduced levels compared to no vaccine, with the VLM vaccines halving amounts, p<0.01 (FIG. 8A). Over half of the animals treated with the VLM DNA vaccines were protected from detectable DNA in the DRG, compared to <20% of the animals who received no vaccine (FIG. 8B).

Analyses of latent DNA detected in the spinal cord showed similar effects with the VLM vaccines significantly reducing the latent DNA load in the spinal cord (p<0.05) and significantly reducing the numbers of animals with detection of DNA in the spinal cord (FIGS. 8C and 8D), with half the animals protected from detectable latency in the spinal cord. However, the gD subunit protein vaccine had a more modest effect on these parameters and did not reach significance for protecting animals from establishing latent infections as detected by DNA in the spinal cord.

5.4 Summary

The VLM DNA vaccine formulations were highly effective against primary disease and virus replication. The VLM DNA vaccine combined with the human chemokine CCL5 had an effect on recurrent virus shedding, not seen with the protein subunit vaccine. Also there were reductions in both on primary and recurrent disease, not seen without CCL5, as well as significant reductions in detection of latent burden, with over half of animals completely protected. Only one animal died in the study and this was in the no vaccine group, as well as two further animals in this group with severe infections preventing sample collection. In comparison, the VLM DNA vaccines were safe, showed infection and disease protection with no adverse effects (summarised in Table 4).

Based on experience of the positive control gD subunit protein vaccine, this was a suboptimal dosing schedule for this model system. Improvement may be found by varying the dosing regimen or using other routes of delivery for these new VLM formulations. Interestingly, since the VLM formulation gave 100% protection from acute disease, single dose delivery may be possible. Furthermore, the positive control gD subunit protein vaccine has already been clinically trialed showing partial efficacy against HSV1 (Belshe et al 2012, 2014) and the results here equal or exceed as noted indicating support for clinical utility. Other cytokines or chemokines could also affect recurrences as shown for the VLM combination with human CCL5.

The cellular recruitment offered by the chemokine showed enhanced effects on recurrences. Cellular immunity is known to affect control of virus reactivation and recurrences and chemokines are known to direct recruitment, activation and migration of immune cells. While the antibody effects can prevent initial infection and can be stimulated by appropriate antigenic presentation as provided by the VLM.

The CCL5 chemokine used here is a human gene tested in the guinea pig model, so effects in human setting likely to further improve outcomes. CCL5 has been well characterised in human cell lines and ex vivo settings. Therefore, the protective effects in the human system are likely to be higher for a chemokine combined with VLM vaccine. This combined with overall efficacy exceeding a positive control with some clinical utility supports further investigation in a clinical setting as a preventative and therapeutic vaccine treatment for HSV2 and further demonstrates utility as both new VLM immunomodulatory treatment in new types of vaccine formulations to provide efficient protection from disease or infection.

TABLE 5

| | | Negative | Positive | | |
|---|---|---|---|---|---|
| | Postulated | control- | control - | | |
| Feature assessed | clinical | No | gD | | VLM + |
| in animal model | correlate | vaccine | protein | VLM | CCL5 |
| Primary disease | Protect from | − | + | ++ | ++ |
| protection | acute disease | | | | |
| Reduce primary | Protect from | − | + | ++ | ++ |
| vaginal replication | acute infection | | | cleared d8 | cleared d8 |
| Reduce recurrent | Protect from | − | + | − | + trend |
| disease | latent virus | | | | |
| | recurrence and | | | | |
| | disease | | | | |
| Reduce | Protect from | − | − | − | + |
| asymptomatic | viral | | | | |
| shedding | transmission | | | | |
| Reduce latent | Protect from | − | + | ++ | ++ |
| viral burden | establishing | | | | |
| | latent infection | | | | |
| | and reduce | | | | |
| | latent virus | | | | |
| | recurrence and | | | | |
| | disease | | | | |

### Example 6: VLM with VIT Immune Modulator

The gD-VLM DNA vaccine was effective as a preventative for the acute HSV2 infection or disease, and was particularly effective against disease recurrences from latency when combined with a chemokine gene, CCL5. Therefore, we tested another chemokine to modify cell immunity to protect from latent virus reactivation.

We have identified a human chemokine, from a human chromosomally integrated endogenous form of human herpesvirus 6A (HHV-6A) genome, referred to herein as iciHHV-6A (Tweedy et al., 2016). We have found a spliced transcript cDNA of a human iciU83A gene from the iciHHV-6A genome that is distinct from the U83A chemokine transcripts from circulating free virus HHV-6A genome, leading to a new chemokine—referred to herein as ‘VIT’ or ‘Virokine Immune Therapeutic’. In the circulating virus, the U83A encodes a chemokine-like molecule, which can mediate immune cell chemotaxis with a unique specificity via interaction with an array of four specific human chemokine receptors, CCR1, CCR4, CCR5 and CCR6 (Catusse et al., 2009; Catusse et al., 2007; Clark et al., 2013; Dewin et al., 2006). This specificity was distinct from that of any other human chemokine or microbial peptide. Compared to the spliced virus U83A, the VIT chemokine gene is extended to a downstream stop codon and comprises an extended truncated product with a unique hydrophobic tag of 8 amino acids which can increase membrane association and stablisation.

We have previously demonstrated that the encoded N-terminal domain dictates specificity of chemokine receptor interactions. While the C-terminal domain retains signaling, if this is removed there is chemokine binding, but no signaling, converting agonist to antagonist activities (Dewin et al., 2006). This could be delineated to a N-terminal 17 amino acid peptide region (Clark et al., 2013). In the VIT molecule, the novel cDNA encodes the intact N-terminal domain, representing the defined receptor specificity but deletes the C-terminal signaling domain instead splicing the small tag.

The U83A genes have an N-terminal poly T tract that vary in length in wild type HHV-6A and in human integrated iciHHV-6A genomes, disrupting gene expression (Tweedy et al 2016). To maintain stable gene expression, this poly T tract is mutated here. This discovery fixes this gene in the functional version, encoding an intact signal sequence so the mature product can be secreted and is introduced here for function.

The specificity reported derived from the maintained N-terminal domain, includes targeting CCR1, 4, 5, 6 and 8 receptors (Catusse et al., 2009; Catusse et al., 2007; Dewin et al., 2006). This unique combination allows targeting of immunesuppresive T-regulator lymphocytes, particularly via CCR4 and CCR6. The human CCR6 is monospecific for human chemokine CCL20, therefore this expands CCR6 receptor interactions as a differentiating property of the VIT molecule. The unique application is in ability to act as an antagonist of these receptors. This is because the C-terminal signaling moiety is no longer present. Antagonism of CCR4 in particular has been demonstrated as a novel mechanism for increasing immunity to a target antigen, by blocking recruitment of T regulator lymphocytes.

The coding sequence for VIT, including endogenous Kozak sequence, is given in SEQ ID NO. 135 and the amino acid sequence for VIT is given in SEQ ID NO. 136. The coding sequence was synthesized and cloned into an expression plasmid pCMV6 in frame with a 5′ promoter and start site for gene transcription from human cytomegalovirus, HCMV, IE gene and 3′ polyadenylation site from SV40 virus for transcript termination.

VIT has been tested as an immunomodulator combined with VLM in the experiments described in the preceding Examples. The formulations and intramuscular immunisations used were as described in the previous Examples for the VLM combined with CCL5 formulation. The VLM+VIT formulation of 250 μg comprised 50 μg each of the five gene expression plasmids, four in VLM and one VIT plasmids.

A summary of the results is shown below.

TABLE 6

| | | | Positive | | |
|---|---|---|---|---|---|
| | Postulated | | control - | | |
| Feature assessed | clinical | VLM + | gD | | VLM + |
| in animal model | correlate | VIT | protein | VLM | CCL5 |
| Primary disease | Protect from | ++ | + | ++ | ++ |
| protection | acute disease | | | | |
| Reduce primary | Protect from | ++ cleared d8 | + | ++ cleared d8 | ++ cleared d8 |
| vaginal replication | acute infection | | | | |
| Reduce recurrent | Protect from | + | + | − | + trend |
| disease | latent virus | | | | |
| | recurrence and | | | | |
| | disease | | | | |
| Reduce | Protect from | + | − | − | + |
| asymptomatic | viral | | | | |
| shedding | transmission | | | | |
| Reduce latent | Protect from | ++ | + | ++ | ++ |
| viral burden | establishing | | | | |
| | latent infection | | | | |
| | and reduce latent | | | | |
| | virus recurrence | | | | |
| | and disease | | | | |

The negative control is scored as ‘-’ and is not shown in the above table.

### Example 7: Inhibition of Recurrent Virus and Disease with DNA Vaccine VLM Combined with VIT Immune Modulator

The experimental set up was the same as in the above examples, with 12 guinea pigs per cohort. Here asymptomatic and symptomatic virus shedding recurrences were evaluated by analyses of positive swabs by quantitative PCR. Almost all animals had detectable levels of virus reactivated, DNA detected above the threshold of 0.5 log copies/microgram of DNA.

VLM on its own or the subunit protein immunisation did not affect recurrent shedding.

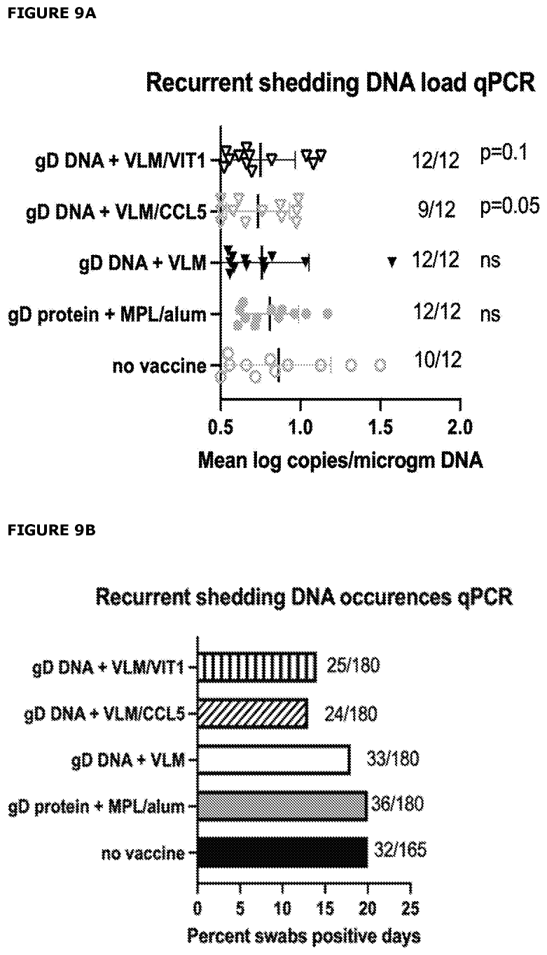

Only immunisation with combinations of VLM and the cytokine genes, CCL5 or VIT showed reductions in the total mean log copies of virus DNA shed (FIG. 9A) and the numbers of actual virus shedding occurrences measured by virus DNA (FIG. 9B). This was significantly reduced with VLM and CCL5 combinations, p<0.05, and showed a trend with VLM and VIT combinations as demonstrated by mean log copies/microgram DNA in individual animals (FIGS. 9A, B).

To analyse effects on individual shedding events, the cumulative daily shedding was evaluated as shown in FIG. 9C. The results show that by one month post-challenge virus infection, all the gD VLM DNA vaccines have halved the number of recurrent virus shedding days compared to no vaccine treatment, while the gD protein+mpl/alum formulation has actually doubled the recurrent shedding days (p=0.018).

However, after two months post-challenge, only the VLM plus cytokine gene formulations, i.e. plus CCL5 or VIT, continue to inhibit virus shedding, now by a third in comparison to the gD protein vaccine. The VLM plus cytokine gene formulations, CCL5 or VIT, compared to the no vaccine treatment continued to show significant inhibition of virus shedding, with no effect by the VLM only gene formulation (p=0.04) (FIG. 9C).

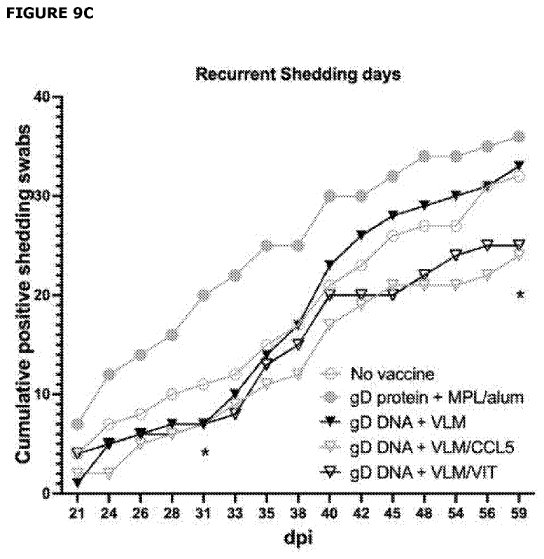

The effects of the immunisations on recurrent lesion days were analysed. The scoring system 1-4, included 1-redness, slight swelling to 4-vesicular-ulcerative lesions. While, none of the VLM vaccinated animals showed any vesicular disease, only the immunisations with VLM+VIT DNA or the gD protein immunisation inhibited recurrent lesion days by over half (FIG. 10A). This is also shown in analyses of the severity of the recurrent lesions. Only immunisations with VLM+VIT DNA or the gD protein significantly reduced recurrent lesions (FIG. 10B).

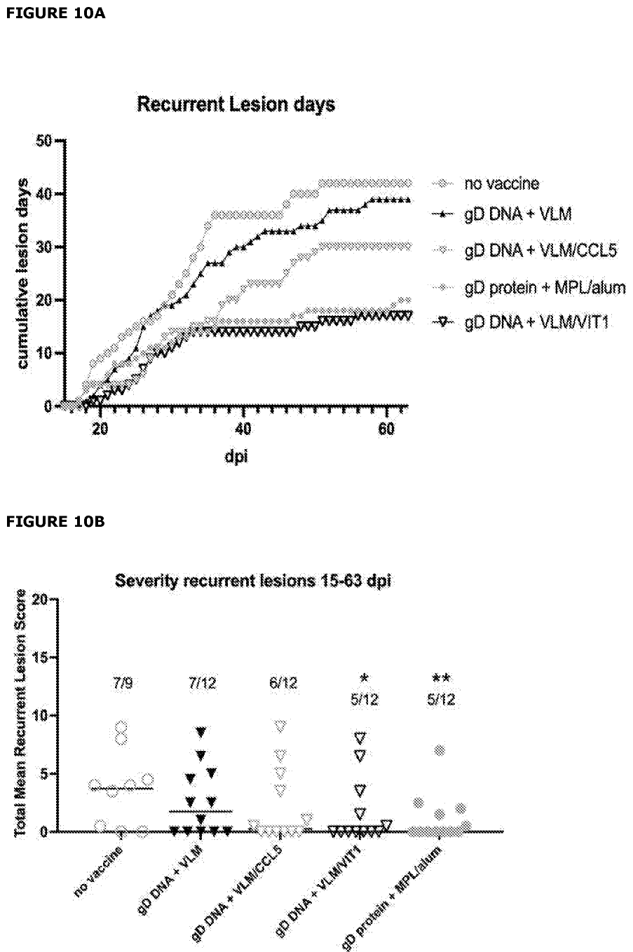

In analyses of establishment of latency within the dorsal root ganglia, all the VLM vaccine formulations were effective at significantly reducing latent DNA load detected (FIG. 11) including the VLM+VIT combination (FIG. 11).

The VLM DNA immunisation prevented acute disease, but only combined with the cytokine genes VIT reduced severity and occurrence of recurrent disease. This was similar to the gD protein.

However, only the VLM plus cytokine gene formulations reduced virus shedding compared to the gD formulation protein immunisation which conversely showed enhanced virus shedding versus no vaccine treatment.

In summary, all the immunisations reduced the virus DNA load at the site of latency in the dorsal root ganglia. However, only the VLM+VIT DNA formulation appeared to inhibit reactivation from latency sufficiently to both reduce virus shedding as well as recurrent disease.

### Example 8: Induction Neutralising Antibody by VLM Vaccine Formulations

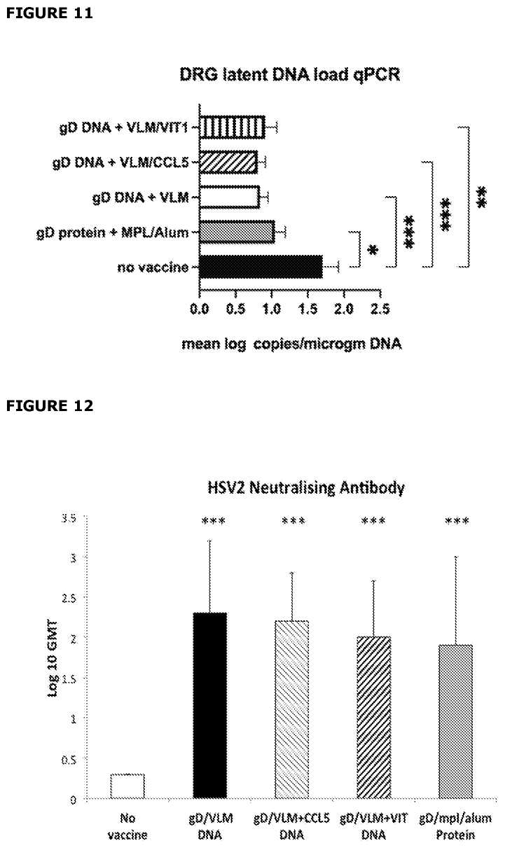

An immune correlate of protection had been established from clinical trial of the gD2 subunit protein combined with MPL/alum as adjuvant (Belshe et al 2014). This showed correlation with protection of infection with HSV-1 with increasing concentrations of antibody in immunised women, as part of a prophylactic vaccine evaluation in this previous clinical trial. Stimulation of antibody responses had also been demonstrated in evaluation of that vaccine in the Guinea Pig model system as used here (Bourne et al 2003) with a three dose immunisation schedule. Evaluation of the VLM DNA vaccines using a shorter two dose immunisation schedule in comparison to the gD2 subunit protein previously clinically trialed, were conducted. Sera were collected prior to virus challenge then assayed for virus neutralisation, using two fold serial dilutions of sera mixed with HSV2 and a suspension of BHK cells, then plated out in culture media to assay for virus plaque formation, as described (Bourne et al 2003). The results showed high levels of neutralising antibody after the immunisations with glycoprotein gD2 protein or the VLM DNA. These were highest in the VLM DNA vaccines, either on their own, or in combination with cytokines CCL5 or VIT. All immunisations induced significant levels of antibodies compared to the no vaccine treatment, which was under the limit of detection, p<0.001, as shown in FIG. 12.

All the immunisations induced significantly raised levels of specific neutralising antibody (FIG. 12) which appeared to correlate with relative effects on reducing acute virus load and disease. However, the different antibody titres were not significantly different between the immunisation treatments and VLM on its own could prevent acute disease.

The differences in effects on recurrent disease are seen with the addition of the chemokine or chemokine-like genes, CCL5 and VIT, to the immunisation schedule and these products can affect leukocyte recruitment. This may skew responses to cellular immunity known to protect from latency and contribute to the high efficacy of the VLM plus cytokine gene formulations in protection from HSV2 infection and disease.

1. A pharmaceutical composition comprising one or more nucleic acid molecules comprising a plurality of immunogen coding regions which collectively encode a plurality of herpesvirus polypeptides, wherein the one or more nucleic acid molecules are capable of expressing the plurality of herpesvirus polypeptides in the form of a herpesvirus fusion complex when introduced into a vertebrate cell, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus; and
wherein the pharmaceutical composition is sterile, and is provided in a sealed sterile container.

2. The pharmaceutical composition of claim 1, wherein each of the plurality of immunogen coding regions has a codon usage, a CpG bias and/or a G+C content which is substantially the same as the codon usage, CpG bias and/or G+C content of the native coding region for the corresponding native full-length herpesvirus polypeptide.

3. The pharmaceutical composition of claim 1, wherein each of the plurality of immunogen coding regions comprises a Kozak sequence which is capable of permitting initiation of translation of the herpesvirus polypeptide in the vertebrate cell with an efficiency which is substantially the same as the efficiency with which the Kozak sequence of the native coding region for the corresponding native full-length herpesvirus polypeptide permits initiation of translation in the vertebrate cell, such as wherein the Kozak sequence of each of the plurality of immunogen coding regions is identical to the Kozak sequence of the native coding region for the corresponding native full-length herpesvirus polypeptide.

4. The pharmaceutical composition of claim 1, wherein each of the immunogen coding regions is operatively linked to a 3′ untranslated region which permits substantially the same degree of mRNA stability of the immunogen coding region or transcript thereof, such as by virtue of comprising an identical 3′ polyadenylation sequence.

5. The pharmaceutical composition of claim 1, wherein the one or more nucleic acid molecules are deoxyribonucleic acid (DNA) polynucleotides, such as plasmid expression vectors or viral vectors; or ribonucleic acid (RNA) polynucleotides, such as viral vectors; and optionally, wherein the one or more nucleic acid molecules are deoxyribonucleic acid (DNA) polynucleotides, and each of the immunogen coding regions is operatively linked to a 5′ promoter, wherein each coding region operatively linked to a 5′ promoter is capable of simultaneous gene expression in the vertebrate cell, such as by virtue of each coding region being linked to an identical 5′ promoter.

6. (canceled)

7. The pharmaceutical composition of claim 1, wherein the gB polypeptide encoded by the immunogen coding region for gB comprises a mutation which stabilises gB polypeptide as a trimer in the fusion conformation, such as a mutation in the fusion associated domain I, such as a substitution at a position corresponding to position 262 of SEQ ID NO 9, such as wherein the substitution is a non-conservative substitution; or a substitution at a position corresponding to position 267 of SEQ ID 91, position 273 of SEQ ID 92, position 202 of SEQ ID 93, position 202 of SEQ ID 94, position 246 of SEQ ID 95, position 193 of SEQ ID 97, position 190 of SEQ ID 98, position 218 of SEQ ID 99, such as wherein the substitution is a non-conservative substitution.

8. The pharmaceutical composition of claim 1, wherein the gB polypeptide encoded by the immunogen coding region for gB comprises a mutation which stabilises gB in a trimer in the prefusion conformation, such as a mutation in the gB structure domains III and/or IV, such as a substitution at a position corresponding to one or more of the substitutions in the encoded polypeptides of SEQ IDs 67 to 90 or 132 to 134; or wherein the gD polypeptide encoded by the immunogen coding region for gD comprises a mutation which lowers interaction with the HVEM receptor, such as a substitution at a position corresponding to position 52 of SEQ ID 64 or SEQ ID 65.

9. The pharmaceutical composition of claim 1, wherein each of the plurality of immunogen coding regions possesses at least 95% sequence identity, such as at least 97% sequence identity, at least 99% sequence identify, at least 99.5% sequence identify or 100% sequence identity to the native coding region for the corresponding native full-length herpesvirus polypeptide; or to a coding region for a variant gB polypeptide which differs from the native coding region for the corresponding native full-length gB polypeptide only in the codon corresponding to position 262 of SEQ ID NO 9 in the encoded variant gB polypeptide.

10. The pharmaceutical composition of claim 1, wherein the gD, gH and gL encoded by the immunogen coding regions have the amino acids sequences of SEQ ID NOs 10, 6 and 7 respectively, and the gB encoded by the immunogen coding region has the amino acids sequence of SEQ ID NO 8 or 9; such as wherein the gD, gH and gL immunogen coding regions have the nucleotide sequences of SEQ ID NOs 5, 1 and 2 respectively, or SEQ ID NOs 18, 15 and 16 respectively; and the gB immunogen coding region has the nucleotide sequence of SEQ ID NO 3, 4 or 17.

11. The pharmaceutical composition of claim 1, wherein the herpesvirus polypeptides encoded by the one or more nucleic acid molecules are limited to those forming the fusion complex, and optionally also a herpesvirus immunomodulator.

12. The pharmaceutical composition of claim 1, wherein the one or more nucleic acid molecules encodes an immunomodulator, optionally CCL5 or VIT, wherein the one or more nucleic acid molecules are capable of expressing the immunomodulator when introduced into the vertebrate cell; and/or wherein the composition comprises an immunomodulator, optionally CCL5 or VIT; and/or wherein the composition comprises an adjuvant; and/or wherein the one or more nucleic acid molecules are supercoiled DNA; and/or wherein the nucleic acid molecules are aggregated with an aggregating agent; and/or wherein the composition comprises bupivacaine.

13. The pharmaceutical composition of claim 1, wherein the one or more nucleic acid molecules encode one or more infectious agent antigens, wherein the one or more nucleic acid molecules are capable of expressing the one or more infectious agent antigens when introduced into the vertebrate cell; and/or wherein the pharmaceutical composition further comprises one or more infectious agent antigens.

14. A pharmaceutical composition comprising a plurality of herpesvirus polypeptides in association with a lipid membrane, wherein the pharmaceutical composition is formed by expressing the plurality of herpesvirus polypeptides in vitro in human cells from one or more nucleic acid molecules comprising a plurality of immunogen coding regions which collectively encode the plurality of herpesvirus polypeptides, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus.

15. The pharmaceutical composition of claim 14, wherein the one or more nucleic acid molecules are as defined in claim 1; and optionally further comprises one or more infectious agent antigens; and optionally wherein the plurality of herpesvirus polypeptides in association with a lipid membrane are provided in the form of membranes, membrane vesicles or whole cells.

16-17. (canceled)

18. A method of therapy, wherein the method comprises administering to a patient in need thereof a pharmaceutical composition, wherein the pharmaceutical composition comprises one or more nucleic acid molecules comprising a plurality of immunogen coding regions which collectively encode a plurality of herpesvirus polypeptides, wherein the one or more nucleic acid molecules are capable of expressing the plurality of herpesvirus polypeptides when introduced into a vertebrate cell, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus; and wherein the pharmaceutical composition is sterile; optionally wherein the pharmaceutical composition is as defined in claim 1.

19. (canceled)

20. The method of therapy according to claim 18, wherein the method is for
(i) inducing an immune response to a herpesvirus; and/or
(ii) preventing or treating a herpesvirus infection, optionally wherein preventing a herpesvirus infection comprises protecting from acute disease and/or infection; protecting from establishing latent infection; protecting from reactivating latent infection and/or viral transmission; and/or protecting from latent viral recurrence and disease; optionally wherein treating a herpesvirus infection comprises protecting from establishing latent infection; protecting from reactivating latent infection and/or viral transmission; and/or protecting from latent viral recurrence and disease; and/or
(iii) inducing an immune response to the one or more infectious agent antigens; and/or
(iv) preventing or treating an infection caused by an infectious agent which comprises the one or more infectious agent antigens.

21-22. (canceled)

23. A method of making the pharmaceutical composition of claim 1, the method comprising formulating the one or more nucleic acid molecules as defined in claim 1 with one or more physiologically acceptable diluents or excipients as a sterile composition and optionally formulating the pharmaceutical composition with one or more infectious agent antigens, and/or an immunomodulator, and/or an adjuvant.

24. A method of making the pharmaceutical composition of claim 14, comprising introducing the one or more nucleic acid molecules as defined in claim 14 into human cells in vitro, allowing the human cells to express the plurality of herpesvirus polypeptides from the one or more nucleic acid molecules, thereby obtaining the plurality of herpesvirus polypeptides in association with a lipid membrane and optionally, wherein the method further comprises collecting membrane vesicles or whole cells comprising the plurality of herpesvirus polypeptides in association with a lipid membrane, and optionally purifying the membrane vesicles or whole cells and/or formulating the pharmaceutical composition with one or more infectious agent antigens, and/or an immunomodulator, and/or an adjuvant.

25-28. (canceled)

29. A method of therapy, wherein the method comprises administering to a patient in need thereof a pharmaceutical composition, wherein the pharmaceutical composition comprises a plurality of herpesvirus polypeptides in association with a lipid membrane, wherein the pharmaceutical composition is formed by expressing the plurality of herpesvirus polypeptides in vitro in human cells from one or more nucleic acid molecules comprising a plurality of immunogen coding regions which collectively encode the plurality of herpesvirus polypeptides, wherein each of the plurality of immunogen coding regions has at least 90% sequence identity to a native coding region for a corresponding native full-length herpesvirus polypeptide from the same herpesvirus species, wherein the plurality of herpesvirus polypeptides are:
(i) gD of herpes simplex virus 2 or herpes simplex virus 1; gE or gI of varicella zoster virus; gp350 or gp42 of Epstein Barr virus; gO selected from genotypes 1-8 of human cytomegalovirus; gO of human herpesvirus 6A; gO of human herpesvirus 6B; gO of human herpesvirus 7; or K8.1 (A/B) of Kaposi's sarcoma associated herpesvirus; and
(ii) gB, gH and gL of the respective cognate human herpesvirus; optionally wherein the pharmaceutical composition is as defined in claim 14.


SEQUENCE LISTING (136 sequences)

SEQ ID NO: 1 - DNA, 2517 bp, Herpes simplex virus type 2 (strain HG52)
  Coding region of gH of HSV2 strain HG52
atgggccccggtctgtgggtggtgatgggggtcctggtgggcgttgccgggggccatgacacgtactggacggagcaaatcgacccgtggtttttgcacggtctggggttggcccgcacgtactggcgcgacacaaacaccgggcgtctgtggttgcccaacacccccgacgccagcgacccccagcgcggacgcttggcgcccccgggcgaactcaacctgactacggcatccgtgcccatgcttcggtggtacgccgagcgcttttgtttcgtgttggtcaccacggccgagtttcctcgggaccccgggcagctgctttacatcccaaagacctatctgctcggccggcctcggaacgcgagcctgcccgagctccccgaggcggggcccacgtcccgtccccccgccgaggtgacccagctcaagggactgtcgcacaaccccggcgcctccgcgctgttgcggtcccgggcctgggtaacattcgcggccgcgccggaccgcgaggggcttacgttcccgcggggagacgacggggcgaccgagaggcacccggacggccgacgcaacgcgccgcccccggggccgcccgcggggacaccgaggcatccgacgacgaacctgagcatcgcgcatctgcacaacgcatccgtgacctggctggccgccaggggcctgctacggactccgggtcggtacgtgtacctctccccgtcggcctcgacgtggcccgtgggcgtctggacgacgggcgggctggcgttcgggtgcgacgccgcgctcgtgcgcgcgcgatacgggaagggcttcatggggctcgtgatatcgatgcgggacagccctccggccgagatcatagtggtgcctgcggacaagaccctcgctcgggtcggaaatccgaccgacgaaaacgcccccgcggtgctccccgggcctccggccggccccaggtatcgcgtctttgtcctgggggccccgacgcccgccgacaacggctcggcgctggacgccctccggcgggtggccggctaccccgaggagagcacgaactacgcccagtatatgtcgcgggcctatgcggagtttttgggggaggacccgggctccggcacggacgcgcgtccgtccctgttctggcgcctcgcggggctgctcgcctcgtcggggtttgcgttcgtcaacgcggcccacgcccacgacgcgattcgcctctccgacctgctgggctttttggcccactcgcgcgtgctggccggcctggccgcccggggagcagcgggctgcgcggccgactcggtgttcctgaacgtgtccgtgttggacccggcggcccgcctgcggctggaggcgcgcctcgggcatctggtggccgcgatcctcgagcgagagcagagcctggtggcgcacgcgctgggctatcagctggcgttcgtgttggacagccccgcggcctatggcgcggtggccccgagcgcggcccgcctgatcgacgccctgtacgccgagtttctcggcggccgcgcgctaaccgccccgatggtccgccgagcgctgttttacgccacggccgtcctccgggcgccgttcctggcgggcgcgccctcggccgagcagcgggaacgcgcccgccggggcctcctcataaccacggccctgtgtacgtccgacgtcgccgcggcgacccacgccgatctccgggccgcgctagccaggaccgaccaccagaaaaacctcttctggctcccggaccacttttccccatgcgcagcttccctgcgcttcgatctcgccgagggcgggttcatcctggacgcgctggccatggccacccgatccgacatcccggcggacgtcatggcacaacagacccgcggcgtggcctccgttctcacgcgctgggcgcactacaacgccctgatccgcgccttcgtcccggaggccacccaccagtgtagcggcccgtcgcacaacgcggagccccggatcctcgtgcccatcacccacaacgccagctacgtcgtcacccacacccccttgccccgcgggatcggatacaagcttacgggcgttgacgtccgccgcccgctgtttatcacctatctcaccgccacctgcgaagggcacgcgcgggagattgagccgaagcggctggtgcgcaccgaaaaccggcgcgacctcggcctcgtgggggccgtgtttctgcgctacaccccggccggggaggtcatgtcggtgctgctggtggacacggatgccacccaacagcagctggcccaggggccggtggcgggcaccccgaacgtgttttccagcgacgtgccgtccgtggccctgttgttgttccccaacggaactgtgattcatctgctggcctttgacacgctgcccatcgccaccatcgcccccgggtttctggccgcgtccgcgctgggggtcgttatgattaccgcggccctggcgggcatccttagggtggtccgaacgtgcgtcccatttttgtggagacgcgaataa

SEQ ID NO: 2 - DNA, 675 bp, Herpes simplex virus type 2 (strain HG52)
  Coding region of HSV strain HG52 glycoprotein gL
atggggttcgtctgtctgtttgggcttgtcgttatgggagcctggggggcgtggggtgggtcacaggcaaccgaatatgttcttcgtagtgttattgccaaagaggtgggggacatactaagagtgccttgcatgcggacccccgcggacgatgtttcttggcgctacgaggccccgtccgttattgactatgcccgcatagacggaatatttcttcgctatcactgcccggggttggacacgtttttgtgggataggcacgcccagagggcgtatctggttaacccctttctctttgcggcgggatttttggaggacttgagtcactctgtgtttccggccgacacccaggaaacaacgacgcgccgggccctttataaagagatacgcgatgcgttgggcagtcgaaaacaggccgtcagccacgcacccgtcagggccgggtgtgtaaactttgactactcacgcactcgccgctgcgtcgggcgacgcgatttacggcctgccaacaccacgtcaacgtgggaaccgcctgtgtcgtcggacgatgaagcgagctcgcagtcgaagcccctcgccacccagccgcccgtcctcgccctttcgaacgcccccccacggcgggtctccccgacgcgaggtcggcgccggcatactcgcctccgacgcaactag

SEQ ID NO: 3 - DNA, 2715 bp, Artificial Sequence
  Mutated coding sequence for glycoprotein gB for HSV2 strain HG52 A784G
atgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcgcgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 4 - DNA, 2721 bp, Herpes simplex virus type 2 (strain HG52)
  Wild-type coding sequence for glycoprotein gB for HSV2 strain HG52 with Kozak sequence
cccgccatgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 5 - DNA, 1182 bp, Herpes simplex virus type 2 (strain HG52)
  Coding sequence of HSV2 strain HG52 glycoprotein gD
atggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgctaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggtgctggtcatcggcggtattgcgttttgggtacgccgccgcgctcagatggcccccaagcgcctacgtctcccccacatccgggatgacgacgcgcccccctcgcaccagccattgttttactag

SEQ ID NO: 6 - protein, 838 aa, Herpes simplex virus type 2 (strain HG52)
  HSV2 strain HG52 glycoprotein gH
MGPGLWVVMGVLVGVAGGHDTYWTEQIDPWFLHGLGLARTYWRDTNTGRLWLPNTPDASDPQRGRLAPPGELNLTTASVPMLRWYAERFCFVLVTTAEFPRDPGQLLYIPKTYLLGRPRNASLPELPEAGPTSRPPAEVTQLKGLSHNPGASALLRSRAWVTFAAAPDREGLTFPRGDDGATERHPDGRRNAPPPGPPAGTPRHPTTNLSIAHLHNASVTWLAARGLLRTPGRYVYLSPSASTWPVGVWTTGGLAFGCDAALVRARYGKGFMGLVISMRDSPPAEIIVVPADKTLARVGNPTDENAPAVLPGPPAGPRYRVFVLGAPTPADNGSALDALRRVAGYPEESTNYAQYMSRAYAEFLGEDPGSGTDARPSLFWRLAGLLASSGFAFVNAAHAHDAIRLSDLLGFLAHSRVLAGLAARGAAGCAADSVFLNVSVLDPAARLRLEARLGHLVAAILEREQSLVAHALGYQLAFVLDSPAAYGAVAPSAARLIDALYAEFLGGRALTAPMVRRALFYATAVLRAPFLAGAPSAEQRERARRGLLITTALCTSDVAAATHADLRAALARTDHQKNLFWLPDHFSPCAASLRFDLAEGGFILDALAMATRSDIPADVMAQQTRGVASVLTRWAHYNALIRAFVPEATHQCSGPSHNAEPRILVPITHNASYVVTHTPLPRGIGYKLTGVDVRRPLFITYLTATCEGHAREIEPKRLVRTENRRDLGLVGAVFLRYTPAGEVMSVLLVDTDATQQQLAQGPVAGTPNVFSSDVPSVALLLFPNGTVIHLLAFDTLPIATIAPGFLAASALGVVMITAALAGILRVVRTCVPFLWRRE

SEQ ID NO: 7 - protein, 224 aa, Herpes simplex virus type 2 (strain HG52)
  HSV2 strain HG52 glycoprotein gL
MGFVCLFGLVVMGAWGAWGGSQATEYVLRSVIAKEVGDILRVPCMRTPADDVSWRYEAPSVIDYARIDGIFLRYHCPGLDTFLWDRHAQRAYLVNPFLFAAGFLEDLSHSVFPADTQETTTRRALYKEIRDALGSRKQAVSHAPVRAGCVNFDYSRTRRCVGRRDLRPANTTSTWEPPVSSDDEASSQSKPLATQPPVLALSNAPPRRVSPTRGRRRHTRLRRN

SEQ ID NO: 8 - protein, 904 aa, Artificial Sequence
  Mutated glycoprotein gB from HSV2 strain HG52 Thr262Ala
  DOMAIN 150..358: Domain I
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGATVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 9 - protein, 904 aa, Herpes simplex virus type 2 (strain HG52)
  Wild-type glycoprotein gB from HSV2 strain HG52
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 10 - protein, 393 aa, Herpes simplex virus type 2 (strain HG52)
  HSV2 strain HG52 glycoprotein gD
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 11 - DNA, 282 bp, Homo sapiens
  Human chemokine CCL5 with endogenous Kozak sequence
ggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 12 - DNA, 1857 bp, Human herpesvirus 6A strain U1102
  GO VARIANT GENE INFRAME WITH HHV-6B Z29 gO gene initiating at second methionine codon
atgcttcacatctcgcgactcggcctttttctgggccttttcgcgatagtcatgcactccgttaatctaataaaatacacgtctgatcccttagaagctttcaaaaccgtcaaccgccacaattggagcgacgaacaaagagaacatttttacgacctccgaaacttatatacaagtttttgtcagacaaatctatccctcgactgcttcactcaaattttaactaacgttttctcttgggacattcgagattcacaatgcaagtctgcggttagcttgtctccattacagaatttaccgcggacagaaatcaaaatagtgctatcctcgacgactgcaaacaaatctatcatcgctagcagtttttctctattttatcttctgtttgccacactatctacatataccgcagatccgccatgcgtagagttactaccatttaaaattctgggagcacagttatttgacataaaactaaccgaagaatccttgcggatggcaatgagcaaattttccaactcgaatctgacacggtcattgacttccttcacgtcaaaaaacttctttaattacaccagctttgtttacttcttgctctataacacaacatcatgcgtcccttcaaatgatcaatatttcaaacagtcgccaaaacctataaatgttaccacttcctttggacgagccatcgtaaactttgattcgatactaactactacaccatcatcgacgtcagcgtctctcacatcaccacatatccctagtaccaacataccaaccccagcacctccccccgtaacaaaaaactctacaaaactgcatacagacaccataaaagttacaccgaacacacccactataacaacgcaaacaacggaaagcatcaaaaaaatagttaaacgttcagattttcctcgacccatgtacaccccaaccgacattccaactcttacaatccgtcttaacgccactattaaaaccgaacaaaacaccgaaaacccaaaaagtccaccaaaaccaacaaattttgaaaataccacaatcagaattcccaaaacccttgagagcgctacagcaacaacaaacgcaacccaaaagatcgaaagcaccaccttcacaacaataggaatcaaggaaattaacggcaatacctattcttcaccaaaaaactctatttatcttaagagcaaatcacagcagagtacaacaaaattcaccgacgccgaacacaccactccgattttaaagtttaccacttggcaaaacacggtacgcacatacatgagtcacaacacagaagtacaaaacatgaccgacaaattccagaggacaaccttgaaatcctcaaacgagctacctaccattcagacgttgtctgtcactccaaaacaaaaactaccgtcgaatgtaactgccaaaactgaagtacacataactaacaacgctttaccatctagtaattcatcatactcaatcactgaagtcactaaagaggtaaagcatactagaatgtcagcgtccactcacgaacagataaaccacacagaaatagcacaaataacaccaattcttaacgctcacacatcggaaaaatcaactacacctcaacggtccttcaccgctgaaacgttcttaacgacatcttcaaagcctaacatcataacctggtcaaacttactaacaacaacgcctaaggaaccattaacgaatacaagtctaaggtggacagatcatatcacaacacagctaacgactagcaatagaactcaatcagccaaactaacaaaagctaacatctcgtcacaaacgactaacatctacccccaaacaatcacgggacgatctacagaggtttaa

SEQ ID NO: 13 - DNA, 2316 bp, Human herpesvirus 6B strain Z29
  Variant gO IN FRAME WITH HHV-6A U1102 GO with SNP
atgcatttggaggtcatcgtccaaccctataaaaaaagcaaatactattttagttacacattctccttgtacaaattcactgtggtcaactcacccgacatgcttcacatctcgcgactcggcctttttctggcccttttcgcgatagtcatgcactccgttaatctaataaaatacacatctgatcccttagaagctttcaaaaccgtcaaccgccacaattggagcgacgaacaaagagaacatttttacgacctccgaaacttgtatacaactttttgtcagagaaatctatccctcgactgcttcactcaaattttaactaacgttttctcttggaacattcgagatttacaatgcaagtctgcggttaacttgtctccattacagaatttaccgcgggcagaaaccaaaatagtgctatcctcaacagctgcaaacaaatctatcgtcgctagcagtttttctctattttatcttctgtttgccacactatctacatataccgcagatccaccatgcgtagagctactaccgtttaaaattctgggaacacagttatttgacataaaactgaccgacgaatccttgcagatggcaatcagtaaattttccaactcgaatctgacacggtcattgactccgttcactcctgaaatcttctttaattacaccagctttgtttacttcttgctctataacacaacatcatgcatccgttcaaatgatcaatatttcgaacattcgccaaaacctataaatgttaccacttcctttggacgagccatcgtaaactttcattcgatactgactacgacgccatcatcgacgccatcatcgacgtcagcgtctatcacatcaccacatatccctagtaccaacacaccaaccccagaaccttcccccgtaacaaaaaactttacagaactgcagacagacaccataaaagttacaccgaacacacccaccataacaacgcaaacaacggaaagcatcaaaaaagtagttaaacgttcagattttcctcgaccgatgtacaccccgactgacattccaactcttacaatccgtcgtaacgccactattaaaaccgaacaaaacaccgaaaaccctaccgaaaacccaaaaagtccaccaaaaccaacaaattttgaaaataccacaatcagaattcccgaaacgtttgagagcactacagtggcaacaaacacgactcaaaagctcgaaagcaccaccttcgcaacaacaataggaatcgaggaaattagcgacaatatctattcttcaccaaaaaactctatttatcttaagagcaaatcacagcagagcacgacaaaattcaccgacaccgaacacaccactccgattttaaagtttaccacttggcaagacgcggcgcgcacatacatgagccacaacacggaagtacaaaacatgaccgaaaattttattaaaatttctcttggagaaacgatgggaatcacgcctaaagaacccacaaatcctacccaacttctcaacgtaaaaaaccaaacagaatacgcaaacgaaacccatagcacagaagtgcagaccgtcaaaaccttcaaagaggacagattccagaggacaactttgaaatcctcaagcgagccacctaccgttcagacgttgtctgtcactccaaaaaaaaaactaccgtcaaatgtaactgccaaaactgaagtacaggtaactaacaatgctttaccatctagtaattcatcacactcaatcactaaagtcaccgaagaaccaaagcaaaatagaatgtcggcgtccactcacggagagatcaaccacacagaaataccacgaatgacaccaattcttaacgctcacacatgggaaaaatcgactacacctcaatggcccttcaccgctgaaacgtccttaacgacatcttcaaagtctgccattctaacctggtcaaacttactaacaacgccaaaggaaccattaacgaatacaagtctaaggtcgacaaatcatatcacaacacagctaacgactagcaacagaactcaatcagccaaactaacaaaagctcacgtctcgtcacaaacgactaacatctacccccaaacaatcacggaacgatctacggacgttaaaaaaaaaagttccacagaaagtcgggaagctaacaaaactctgcctggaaacgactaccgcgtcaccgataaaaactcacacaaccatccagataacctcactaccaaagcatactcaacccaaaatgcaacgcattacacatacaacgaacgacacgatttaaacaacacagatagcacataa

SEQ ID NO: 14 - DNA, 692 bp, Human herpesvirus 8 K8.1 strain GK18
  HHV8 K8.1 ORF variant predicted read through
atgagttccacacagattcgcacagaaatccctgtggcgctcctaatcctatgcctttgtctggtggcgtgccatgccaattgtcccacgtatcgttcgcatttgggattctggcaagagggttggagtggacaggtttatcaggactggctaggcaggatgaactgttcctacgagaatatgacggccctagaggccgtctccctaaacgggaccagactagcagctggatctccgtcgagtgagtatccaaatgtctccgtatctgttgaagatacgtctgcctctgggtctggagaagatgcaatagatgaatcggggtcgggggaggaagagcgtcccgtgacctcccacgtgacttttatgacacaaagcgtccaggccaccacagaactgaccgatgccttaatatcagccttttcaggatcatattcatctggggaaccatccaggaccacgcgaattcgcgtatcaccggtcgcagaaaacggcagaaatagtggtgctagtaaccgcaaaaccataaaagtaaataaacgtgtttattgttcacatgataaagagtggtactctttactggtttgggggttgggttgtggcgtggtggctggtccgcggttcagtcatcaacccccgcccgtgttgtcgaggctcctcttcgtcgcctgttattggcaccaggaggcggttta

SEQ ID NO: 15 - DNA, 2523 bp, Herpes simplex virus type 2 (strain HG52)
  Coding region of gH of HSV2 strain HG52 with Kozak site
acgaccatgggccccggtctgtgggtggtgatgggggtcctggtgggcgttgccgggggccatgacacgtactggacggagcaaatcgacccgtggtttttgcacggtctggggttggcccgcacgtactggcgcgacacaaacaccgggcgtctgtggttgcccaacacccccgacgccagcgacccccagcgcggacgcttggcgcccccgggcgaactcaacctgactacggcatccgtgcccatgcttcggtggtacgccgagcgcttttgtttcgtgttggtcaccacggccgagtttcctcgggaccccgggcagctgctttacatcccaaagacctatctgctcggccggcctcggaacgcgagcctgcccgagctccccgaggcggggcccacgtcccgtccccccgccgaggtgacccagctcaagggactgtcgcacaaccccggcgcctccgcgctgttgcggtcccgggcctgggtaacattcgcggccgcgccggaccgcgaggggcttacgttcccgcggggagacgacggggcgaccgagaggcacccggacggccgacgcaacgcgccgcccccggggccgcccgcggggacaccgaggcatccgacgacgaacctgagcatcgcgcatctgcacaacgcatccgtgacctggctggccgccaggggcctgctacggactccgggtcggtacgtgtacctctccccgtcggcctcgacgtggcccgtgggcgtctggacgacgggcgggctggcgttcgggtgcgacgccgcgctcgtgcgcgcgcgatacgggaagggcttcatggggctcgtgatatcgatgcgggacagccctccggccgagatcatagtggtgcctgcggacaagaccctcgctcgggtcggaaatccgaccgacgaaaacgcccccgcggtgctccccgggcctccggccggccccaggtatcgcgtctttgtcctgggggccccgacgcccgccgacaacggctcggcgctggacgccctccggcgggtggccggctaccccgaggagagcacgaactacgcccagtatatgtcgcgggcctatgcggagtttttgggggaggacccgggctccggcacggacgcgcgtccgtccctgttctggcgcctcgcggggctgctcgcctcgtcggggtttgcgttcgtcaacgcggcccacgcccacgacgcgattcgcctctccgacctgctgggctttttggcccactcgcgcgtgctggccggcctggccgcccggggagcagcgggctgcgcggccgactcggtgttcctgaacgtgtccgtgttggacccggcggcccgcctgcggctggaggcgcgcctcgggcatctggtggccgcgatcctcgagcgagagcagagcctggtggcgcacgcgctgggctatcagctggcgttcgtgttggacagccccgcggcctatggcgcggtggccccgagcgcggcccgcctgatcgacgccctgtacgccgagtttctcggcggccgcgcgctaaccgccccgatggtccgccgagcgctgttttacgccacggccgtcctccgggcgccgttcctggcgggcgcgccctcggccgagcagcgggaacgcgcccgccggggcctcctcataaccacggccctgtgtacgtccgacgtcgccgcggcgacccacgccgatctccgggccgcgctagccaggaccgaccaccagaaaaacctcttctggctcccggaccacttttccccatgcgcagcttccctgcgcttcgatctcgccgagggcgggttcatcctggacgcgctggccatggccacccgatccgacatcccggcggacgtcatggcacaacagacccgcggcgtggcctccgttctcacgcgctgggcgcactacaacgccctgatccgcgccttcgtcccggaggccacccaccagtgtagcggcccgtcgcacaacgcggagccccggatcctcgtgcccatcacccacaacgccagctacgtcgtcacccacacccccttgccccgcgggatcggatacaagcttacgggcgttgacgtccgccgcccgctgtttatcacctatctcaccgccacctgcgaagggcacgcgcgggagattgagccgaagcggctggtgcgcaccgaaaaccggcgcgacctcggcctcgtgggggccgtgtttctgcgctacaccccggccggggaggtcatgtcggtgctgctggtggacacggatgccacccaacagcagctggcccaggggccggtggcgggcaccccgaacgtgttttccagcgacgtgccgtccgtggccctgttgttgttccccaacggaactgtgattcatctgctggcctttgacacgctgcccatcgccaccatcgcccccgggtttctggccgcgtccgcgctgggggtcgttatgattaccgcggccctggcgggcatccttagggtggtccgaacgtgcgtcccatttttgtggagacgcgaataa

SEQ ID NO: 16 - DNA, 681 bp, Herpes simplex virus type 2 (strain HG52)
  Coding region of HSV strain HG52 glcyoprotein gL with Kozak sequence
ctcgctatggggttcgtctgtctgtttgggcttgtcgttatgggagcctggggggcgtggggtgggtcacaggcaaccgaatatgttcttcgtagtgttattgccaaagaggtgggggacatactaagagtgccttgcatgcggacccccgcggacgatgtttcttggcgctacgaggccccgtccgttattgactatgcccgcatagacggaatatttcttcgctatcactgcccggggttggacacgtttttgtgggataggcacgcccagagggcgtatctggttaacccctttctctttgcggcgggatttttggaggacttgagtcactctgtgtttccggccgacacccaggaaacaacgacgcgccgggccctttataaagagatacgcgatgcgttgggcagtcgaaaacaggccgtcagccacgcacccgtcagggccgggtgtgtaaactttgactactcacgcactcgccgctgcgtcgggcgacgcgatttacggcctgccaacaccacgtcaacgtgggaaccgcctgtgtcgtcggacgatgaagcgagctcgcagtcgaagcccctcgccacccagccgcccgtcctcgccctttcgaacgcccccccacggcgggtctccccgacgcgaggtcggcgccggcatactcgcctccgacgcaactag

SEQ ID NO: 17 - DNA, 2721 bp, Artificial Sequence
  Coding sequence for mutated glycoprotein gB for HSV2 strain HG52 with Kozak sequence A784G
cccgccatgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcgcgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 18 - DNA, 1188 bp, Herpes simplex virus type 2 (strain HG52)
  Coding sequence of HSV2 strain HG52 glycoprotein gD with own Kozak sequence
cacggcatggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgctaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggtgctggtcatcggcggtattgcgttttgggtacgccgccgcgctcagatggcccccaagcgcctacgtctcccccacatccgggatgacgacgcgcccccctcgcaccagccattgttttactag

SEQ ID NO: 19 - DNA, 2724 bp, Human gammaherpesvirus 4
  NC_007605.1c79865-77142 Human gammaherpesvirus 4tp1 genome gp350 BLLF1
atggaggcagccttgcttgtgtgtcagtacaccatccagagcctgatccatctcacgggtgaagatcctggttttttcaatgttgagattccggaattcccattttaccccacatgcaatgtttgcacggcagatgtcaatgtaactatcaatttcgatgtcgggggcaaaaagcatcaacttgatcttgactttggccagctgacaccccatacgaaggctgtctaccaacctcgaggtgcatttggtggctcagaaaatgccaccaatctctttctactggagctccttggtgcaggagaattggctctaactatgcggtctaagaagcttccaattaacgtcaccaccggagaggagcaacaagtaagcctggaatctgtagatgtctactttcaagatgtgtttggaaccatgtggtgccaccatgcagaaatgcaaaaccccgtgtacctgataccagaaacagtgccatacataaagtgggataactgtaattctaccaatataacggcagtagtgagggcacaggggctggatgtcacgctacccttaagtttgccaacgtcagctcaagactcgaatttcagcgtaaaaacagaaatgctcggtaatgagatagatattgagtgtattatggaggatggcgaaatttcacaagttctgcccggagacaacaaatttaacatcacctgcagtggatacgagagccatgttcccagcggcggaattctcacatcaacgagtcccgtggccaccccaatacctggtacagggtatgcatacagcctgcgtctgacaccacgtccagtgtcacgatttcttggcaataacagtatcctgtacgtgttttactctgggaatggaccgaaggcgagcgggggagattactgcattcagtccaacattgtgttctctgatgagattccagcttcacaggacatgccgacaaacaccacagacatcacatatgtgggtgacaatgctacctattcagtgccaatggtcacttctgaggacgcaaactcgccaaatgttacagtgactgccttttgggcctggccaaacaacactgaaactgactttaagtgcaaatggactctcacctcggggacaccttcgggttgtgaaaatatttctggtgcatttgcgagcaatcggacatttgacattactgtctcgggtcttggcacggcccccaagacactcattatcacacgaacggctaccaatgccaccacaacaacccacaaggttatattctccaaggcacccgagagcaccaccacctcccctaccttgaatacaactggatttgctgatcccaatacaacgacaggtctacccagctctactcacgtgcctaccaacctcaccgcacctgcaagcacaggccccactgtatccaccgcggatgtcaccagcccaacaccagccggcacaacgtcaggcgcatcaccggtgacaccaagtccatctccatgggacaacggcacagaaagtaaggcccccgacatgaccagctccacctcaccagtgactaccccaaccccaaatgccaccagccccaccccagcagtgactaccccaaccccaaatgccaccagccccaccccagcagtgactaccccaaccccaaatgccaccagccccaccttgggaaaaacaagtcctacctcagcagtgactaccccaaccccaaatgccaccagccccaccttgggaaaaacaagccccacctcagcagtgactaccccaaccccaaatgccaccagccccaccttgggaaaaacaagccccacctcagcagtgactaccccaaccccaaatgccaccggccctactgtgggagaaacaagtccacaggcaaatgccaccaaccacaccttaggaggaacaagtcccaccccagtagttaccagccaaccaaaaaatgcaaccagtgctgttaccacaggccaacataacataacttcaagttcaacctcttccatgtcactgagacccagttcaaacccagagacactcagcccctccaccagtgacaattcaacgtcacatatgcctttactaacctccgctcacccaacaggtggtgaaaatataacacaggtgacaccagcctctatcagcacacatcatgtgtccaccagttcgccagcaccccgcccaggcaccaccagccaagcgtcaggccctggaaacagttccacatccacaaaaccgggggaggttaatgtcaccaaaggcacgcccccccaaaatgcaacgtcgccccaggcccccagtggccaaaagacggcggttcccacggtcacctcaacaggtggaaaggccaattctaccaccggtggaaagcacaccacaggacatggagcccggacaagtacagagcccaccacagattacggcggtgattcaactacgccaagaccgagatacaatgcgaccacctatctacctcccagcacttctagcaaactgcggccccgctggacttttacgagcccaccggttaccacagcccaagccaccgtgccagtcccgccaacgtcccagcccagattctcaaacctctccatgctagtactgcagtgggcctctctggctgtgctgacccttctgctgctgctggtcatggcggactgcgcctttaggcgtaacttgtctacatcccatacctacaccaccccaccatatgatgacgccgagacctatgtataa

SEQ ID NO: 20 - DNA, 672 bp, Human gammaherpesvirus 4
  NC_007605.1c89828-89157 Human gammaherpesvirus 4tp1 genome, gp42 BZLF2
atggtttcatttaagcaggtgagggtgccattgtttaccgccatcgcacttgttattgttctactcctggcatactttttgccacccagggtaagaggaggagggcgggtggcagccgcggccatcacctgggtacccaaaccaaatgtagaggtctggccggtggatcctccaccgccggttaactttaacaagacggccgagcaggagtatggggacaaagaggtaaaactgccacattggacacccaccctgcacacatttcaggtaccccaaaactataccaaagctaactgtacatactgcaacaccagagaatacacattttcatataaaggatgctgtttttatttcaccaaaaagaagcacacctggaatgggtgtttccaagcctgtgcagagctatatccatgcacttatttttatgggccaacgcccgatattctacctgtggtaactagaaatctgaatgccattgagtccctttgggtcggggtgtacagggtgggagaagggaactggacatcattagatggggggacttttaaggtttatcaaatttttggctctcattgtacatatgtcagcaaatttagtacagttccagtctcacaccatgagtgttcattccttaaaccatgtttatgtgtcagtcaaagatcaaatagctaa

SEQ ID NO: 21 - DNA, 2574 bp, Human gammaherpesvirus 4
  NC_007605.1c158864-156291 Human gammaherpesvirus 4tp1 genome gB BALF4
atgactcggcgtagggtgctaagcgtggtcgtgctgctagccgccctggcgtgccgtctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagaactaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacacgctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgcccccagccccatccgcggcccgcgggagcacccccgccgccgttctgaggcgtcggaggcgggatgcggggaacgccaccacaccggtgccccccacggcccccgggaagtccctgggcaccctcaacaatcccgccaccgtccagatccaatttgcctacgactccctgcgccgccagatcaaccgcatgctgggagaccttgcgcgggcctggtgcctggagcagaagaggcagaacatggtgctgagagaactaaccaagattaatccaaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccactttaaaaccatcgagctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagttttaa

SEQ ID NO: 22 - DNA, 2121 bp, Human gammaherpesvirus 4
  NC_007605.1c130747-128627 Human gammaherpesvirus 4tp1 genome gH BXLF2
atgcagttgctctgtgttttttgcctggtgttgctatgggaggtgggggctgccagcctcagcgaggttaagctgcacctggacatagaggggcatgcttcgcattacaccatcccatggaccgaactgatggcaaaggtcccaggccttagcccagaggcgctgtggagagaggcaaatgtcaccgaagatttggcgtctatgcttaaccgctacaagttaatttacaagacgtctggtacccttggtattgcgctggccgagcctgtcgatatccctgctgtctctgaaggatccatgcaagtggatgcatctaaggtccatcccggagtcattagcggcctgaattcccctgcctgcatgcttagtgccccccttgagaagcagctcttctactatattggcaccatgctgcccaacacgcggccacacagctatgtcttttatcagctgcgctgtcacttgtcttatgtggccctgtccatcaacggggacaagtttcagtacacgggggccatgacttctaaatttctgatgggcacctacaagcgagtgaccgagaagggagatgagcatgtgttgagcctggtctttggcaagacgaaggacctgccggatctgagggggccttttagttacccatccttaaccagtgcccaaagcggggactattccctggtgattgttacaacctttgtgcattatgccaactttcacaactactttgtacccaacctgaaggatatgttttcccgagccgtcaccatgacagccgccagctacgctcgctacgttctccagaaactggtcctgctggagatgaagggaggctgccgggagccggaactggacacggaaacgctgactaccatgtttgaggtttctgtggccttctttaaggtgggtcatgctgtgggtgagactggcaatggctgcgtggacctccgctggttggccaagagcttctttgagctgactgtcctgaaagacatcatcggcatatgttatggggccactgtcaagggcatgcaatcctacgggctggagcgcttggccgccatgctgatggccacggtcaagatggaggagctgggtcacctgactactgagaaacaggagtacgcgctgaggttagccaccgtcggctaccccaaggccggggtttacagtggcctcattggaggcgccacatctgtgcttctctcggcctacaaccgccacccccttttccagcccctgcataccgtgatgagagagaccctgtttatcggcagccacgtggtgctacgcgagttgcggctgaacgtgactacccaggggcccaaccttgccctataccaactgctgtccaccgccctgtgctcggccctagagattggggaggttttgcgggggctagccctggggacagagagcgggctcttctcaccgtgctacctcagcctacgatttgacctcacacgagacaagctgctgagcatggccccccaggaggcaacgctggaccaggcggccgtttcaaatgctgtggatgggtttcttgggcggctctctttggagcgagaagacagggatgcgtggcatctccccgcctacaaatgcgtggacaggctcgacaaagttctgatgattatcccgctcatcaatgtgacattcataatctctagtgaccgtgaggtccgaggctcggcgctatacgaggccagcaccacctatctcagcagctctctctttctctcccccgttataatgaataaatgttcgcagggtgctgtggctggggagccccgccagattccaaagatccagaattttaccaggacgcagaaatcctgcattttttgtggctttgccctgctcagttatgatgaaaaggaaggcctggaaactacaacctacatcacctcccaggaagtccaaaactccatcttgagctccaactactttgattttgacaacctccacgttcactatctgctgctgaccaccaacgggactgtcatggaaattgcgggcctgtatgaagaaagagcacacgttgttttggcaataatcctgtactttattgcttttgctctgggtatctttctggttcacaagattgttatgtttttcctttag

SEQ ID NO: 23 - DNA, 672 bp, Human gammaherpesvirus 4; identical to SEQ ID NO: 20
  NC_007605.1c89828-89157 Human gammaherpesvirus 4tp1 genome, gL BZLF2

SEQ ID NO: 24 - DNA, 2661 bp, Human herpesvirus 4
  NC_009334.1c79936-77276 Human herpesvirus 4tp2 genome, gp350, BLLF1
atggaggcagccttgcttgtgtgtcagtacaccatccagagccttatccaactcacgcgtgatgatcctggttttttcaatgttgagattctggaattcccattttacccagcgtgcaatgtttgcacggcagatgtcaatgcaactatcaatttcgatgtcgggggcaaaaagcataaacttaatcttgactttggcctgctgacaccccatacaaaggctgtctaccaacctcgaggtgcatttggtggctcagaaaatgccaccaatctctttctactggagctccttggtgcaggagaattggctctaactatgcggtctaagaagcttccaattaacatcaccaccggagaggagcaacaagtaagcctggaatctgtagatgtctactttcaagatgtgtttggcaccatgtggtgccaccatgcagaaatgcaaaacccagtatacctaataccagaaacagtgccatacataaagtgggataactgtaattctaccaatataacggcagtagtaagggcacaggggctggatgtcacgctacccttaagtttgccaacatcagctcaagactcgaatttcagcgtaaaaacagaaatgctcggtaatgagatagatattgagtgtattatggaggatggcgaaatttcacaagttctgcccggagacaacaaatttaacatcacctgcagtggatacgagagccatgttcccagcggcggaattctcacatcaacgagtcccgtggccaccccaatacctggtacagggtatgcatacagcctgcgtctgacaccacgtccagtgtcacgatttcttggcaataacagtatactgtacgtgttttactctgggaatggaccgaaggcgagcgggggagattactgcattcagtccaacattgtgttctctgatgagattccagcttcacaggacatgccgacaaacaccacagacatcacatatgtgggtgacaatgctacctattcagtgccaatggtcacttctgaggacgcaaactcgccaaatgttacagtgactgccttttgggcctggccaaacaacactgaaactgactttaagtgcaaatggactctcacctcggggacaccttcgggttgtgaaaatatttctggtgcatttgcgagcaatcggacatttgacattactgtctcgggtcttggcacggcccccaagacactcattatcacacgaacggctaccaatgccaccacaacaacccacaaggttatattctccaaggcacccgagagcaccaccacctcccctaccttgaatacaactggatttgctgctcccaatacaacgacaggtctacccagctctactcacgtgcctaccaacctcaccgcacctgcaagcacaggccccactgtatccaccgcggatgtcaccagcccaacaccagccggcacaacgtcaggcgcatcaccggtgacaccaagtccatctccacgggacaacggcacagaaagtaaggcccccgacatgaccagccccacctcagcagtgactaccccaaccccaaatgccaccagccccaccccagcagtgactaccccaaccccaaatgccaccagccccaccttgggaaaaacaagtcccacctcagcagtgactaccccaaccccaaatgccaccagccccaccccagcagtgactaccccaaccccaaatgccaccatccccaccttgggaaaaacaagtcccacctcagcagtgactaccccaaccccaaatgccaccagccctaccgtgggagaaacaagtccacaggcaaataccaccaaccacacattaggaggaacaagttccaccccagtagttaccagcccaccaaaaaatgcaaccagtgctgttaccacaggccaacataacataacttcaagttcaacctcttccatgtcactgagacccagttcaatctcagagacactcagcccctccaccagtgacaattcaacgtcacatatgcctttactaacctccgctcacccaacaggtggtgaaaatataacacaggtgacaccagcctctaccagcacacatcatgtgtccaccagttcgccagcgccccgcccaggcaccaccagccaagcgtcaggccctggaaacagttccacatccacaaaaccgggggaggttaatgtcaccaaaggcacgccccccaaaaatgcaacgtcgccccaggcccccagtggccaaaagacggcggttcccacggtcacctcaacaggtggaaaggccaattctaccaccggtggaaagcacaccacaggacatggagcccggacaagtacagagcccaccacagattacggcggtgattcaactacgccaagaacgagatacaatgcgaccacctatctacctcccagcacttctagcaaactgcggccccgctggacttttacgagcccaccggttaccacagcccaagccaccgtgcctgtcccgccaacgtcccagcccagattctcaaacctctccatgctagtactgcagtgggcctctctggctgtgctgacccttctgctgctgctggtcatggcggactgcgccttcaggcgtaacttgtcgacatcccatacctacaccaccccaccatatgatgacgccgagacctatgtataa

SEQ ID NO: 25 - DNA, 672 bp, Human herpesvirus 4
  NC_009334.1c90630-89959 Human herpesvirus 4tp2 genome, gp42
atggtttcgtttaagcaggtgagggtgccattgtttaccgccatcgcacttgttattgttctactcctggcatactttttgccacccagggtaagaggaggagggcgggtgtcggcagcggccatcacctgggtacccaaaccaaatgtagaggtctggccggtggatcctccaccgccggttaactttaacaagacggccgagcaggagtatggggacaaagagataaaactgccacattggacacccaccctgcacacgtttcaggtacccaaaaactataccaaagctaactgtacatactgcaacacaagagaatacacattttcatataaagaacgctgtttttatttcaccaaaaagaagcacacctggaatgggtgtttccaagcctgtgcagagctatatccatgcacttatttttatgggccaacgcccgatattctacctgtggtaactagaaatctgaatgccattgagtccctttgggtcggggtgtacagggtgggagaagggaactggacatcattagatggggggacttttaaggtttatcaaatttttggctctcattgtacatatgtcagcaaatttagtacagttccagtctcacaccatgagtgttcattccttaaaccatgtttatgtgtcagtcaaagatcaaatagctaa

SEQ ID NO: 26 - DNA, 2574 bp, Human herpesvirus 4
  NC_009334.1c160348-157775 Human herpesvirus 4tp2 genome, glycoprotein B BALF4
atgactcggcgtagggtgctaagcgtggtcgtgctgctagccgccctggcgtgccgcctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagaactaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacaccctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgccccccgccccacccgcggcccgcgggagcacctccgccgccgttctgaggcgccggaggcgggatgcggggaatgccaccacaccggtgccccccgcggcccccgggaagtccctgggcaccctcaacaatcccgccaccgtccagatccaatttgcctacgattccctgcgccgccagatcaaccgcatgctgggagacctcgcgcgggcctggtgcctggagcagaagaggcagaacatggtgctgagagaactaaccaagattaatcccaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccactttaaaaccatcgagctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagttttaa

SEQ ID NO: 27 - DNA, 2121 bp, Human herpesvirus 4
  NC_009334.1c131574-129454 Human herpesvirus 4tp2 genome, Glycoprotein H BXLF2 gp85
atgcagttgctctgtgttttttgcctggtgttgctatgggaggtgggggctgccagtctcagcgaggttaagctgcacctggacatagaggggcatgcttcgcattacaccatcccatggaccgaactgatggcaaaggtcccaggccttagcccagaggcgctgtggagagaggcaaatgtcaccgaagatttggcgtctatgcttaaccgctacaagttaatttacaagacgtctggtacccttggtattgcgctggccgagcctgtcgatatccctgctgtctctgaaggatccatgcaagtggatgcatctaaggtccatcccggagtcattagcggcctgaattcccctgcctgcatgcttagtgccccccttgagaagcagctcttctactatattggcaccatgctgcccaacacgcggccacacagctatgtcttttatcagctgcgctgtcacttgtcttatgtggccctgtccatcaacggggacaagtttcagtacacgggggccatgacttctaaatttctgatgggcacctacaagcgagtgaccgagaagggagatgagcatgtgttgagcctgatctttggcaagacgaaggacctgccggatctgagggggccttttagttacccatccttaaccagtgcccaaagcggggactattccctggtgattgttacaacctttgtgcattatgccaactttcacaactactttgtacccaacctgaaggatatgttttcccgagccgtcaccatgacagccgccagctacgctcgctacgttctccagaaactggtcctgctggagatgaagggaggctgccgggagccagaactggacacggaaacgctgactaccatgtttgaggtttctgtggccttctttaaggtgggtcatgccgtgggtgagactggcaatggctgcgtggacctccgctggttggccaagagcttctttgagctgactgtcctgaaagacatcatcggcatatgttatggggccactgtcaagggcatgcaatcctacgggctggagcgcttggccgccgtgctgatggccacggtcaagatggaggagcttggtcacctgacgactgagaaacaggagtacgcgctgaggttagccaccgtcggctaccccaaggccggggtttacagtggcctcattggaggcgccacatctgtgcttctctcggcctacaaccgccacccccttttccagcccctgcataccgtgatgagagagaccctgtttatcggcagccacgtggtgctacgcgagttgcggctgaacgtgactacccaggggcccaaccttgccctataccaactgctgtccaccgccctgtgctcggccctagagattggggaggttttgcgggggctagccctggggacggagagcgggctcttctcaccgtgctacctcagcctacgatttgacctcacacgagacaagctgctgagcatggccccccaggaggcaatgctggaccaggcggccgtttcaaatgctgtggatgggtttcttgggcgtctctctttggagcgagaagacagggatgcgtggcatctccccgcctacaaatgcgtggacaggctcgacaaagttctgatgattatcccgctcatcaacgtgacattcataatctctagtgaccgtgaggtccgaggctcggcgctatacgaggccagcaccacctatctcagcagctctctctttctctcccccgttataatgaataaatgttcgcagggtgctgtggctggggagccccgccagattccaaagatccagaattttaccaggacgcagaaatcctgcattttttgtggctttgccctgctcagttatgatgaaaaggaaggcctggaaactacaacctacatcacctcccaggaagtccaaaactccatcttgagctccaactactttgattttgacaacctccacgttcactatctgctgctgaccaccaacgggactgtcatggaaattgcgggcctgtatgaagaaagagcacacgttgttttggcaataatcctgtactttattgcttttgctctgggtatctttctggttcacaagattgttatgtttttcctttag

SEQ ID NO: 28 - DNA, 414 bp, Human herpesvirus 4
  NC_009334.198500-98913 Human herpesvirus 4tp2 genome, glycoprotein gL BKRF2
atgcgtactgttggtgtatttctggccacctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagctggtcatcagccgctgcgcaaacggactcaacgtggtctccttctttatctccatcctgaagcgaagcagctccgccctcacgggccatctccgtgagttgttaaccaccctggagactctttacggttcattctcagtggaagacctgtttggtgccaacttaaacagatacgcatggcatcgcgggggctag

SEQ ID NO: 29 - DNA, 2724 bp, Human herpesvirus 5 strain Merlin
  NC_006273.2c84789-82066 Human herpesvirus 5 strain Merlin genome UL55 gB
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctga

SEQ ID NO: 30 - DNA, 2229 bp, Human herpesvirus 5 strain Merlin
  NC_006273.2c111452-109224 Human herpesvirus 5 strain Merlin genome, gH UL75
atgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacctgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcatgatgtccgtctacgcgctatcggccatcatcggcatctatctgctctaccgcatgctcaagacatgctga

SEQ ID NO: 31 - DNA, 837 bp, Human herpesvirus 5 strain Merlin
  NC_006273.2c165858-165022 Human herpesvirus 5 strain Merlin genome, gL UL115
atgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctaa

SEQ ID NO: 32 - DNA, 1419 bp, Human herpesvirus 5 strain Merlin
  NC_006273.2c108848-107430 Human herpesvirus 5 strain Merlin genome, gO5 UL74
atggggaaaaaagagatgataatggtgaaaggcattcctaaaattatgctcctgatctctataacgttcttgctcctttccctcataaattgtaatgtattggtaaactccagaggaacaagacgttcctggccgtataccgtgctatcttatcgaggtaaagagattctgaagaaacagaaggaagatatcttaaaacgattgatgtctacatcatctgacggataccggtttttaatgtaccccagtcagcaaaaatttcatgccatcgttattagcatggataaatttcctcaagactacattttagcgggtcccattagaaatgatagcattacccatatgtggtttgacttttacagtactcaactccgaaaaccagccaagtacgtatattccgaatataatcacacggcccacaaaataacgttacgacccccaccttgcggcacagtgccttctatgaactgcctatccgaaatgttaaatgtttccaaacgcaatgataccggcgaaaaaggttgcggtaatttcaccacgtttaatcctatgtttttcaacgtaccacgttggaacacaaaactgtacataggttccaacaaagtcaacgtggatagtcagacaatctactttttgggcctaaccgccctacttttacgatacgcgcaacgtaactgcactcgcagtttctacctggttaacgccatgagccgaaatttattccgcgttcccaagtatattaacggcaccaagttgaaaaacactatgcgaaaactcaaacgtaaacaagcgcttgtcaaagaacaaccacaaaaaaagaataagaaatctcaaagtactactacgccatatctttcctatacaacgtctaccgctttcaacgtcaccactaacgtgacttatagtgctaccgctgctgtaacgcgggttgccacatctacgacaggttatcgtcctgatagtaactttatgaaatccattatggccacgcagttaagagatctcgcaacatgggtatatactactctgcggtatcggaatgaacccttttgtaaaccagaccgtaaccgtaccgccgtgtcagaatttatgaaaaacacgcacgtactgattcgtaacgaaacgccgtacactatttatggcactcttgacatgagctccttatattacaacgaaaccatgtccgtggaaaacgaaacggcttccgataataacgaaactacacctacgtcaccatcgacgaggtttcagagaacgttcatagatcccctatgggactatctagactcgctgctgtttctagataaaatccgtaactttagcctccagttacccgcgtatggaaatcttaccccgccggaacaccgccgggctgcaaatctatccaccctcaatagcctttggtggtggtcgcagtaa

SEQ ID NO: 33 - DNA, 1401 bp, Human herpesvirus 5 strain Merlin
  NC_001347.5c108456-107056 Human herpesvirus 5 genome AD169 varUK gO1a UL74
atggggagaaaagagatgatggtgagagacgtccctaagatggtgtttctaatatctatatctttcttgcttgtttctttcataaactgtaaagttatgtcaaaagcgctttataatcgtccttggaggggcttggtactgtctaagataggcaaatataaattagatcagcttaagttagaaattttgagacaactagaaacgactatttctacaaaatacaatgtaagtaaacaaccggttaaaaatctcactatgaacatgacagagtttccacaatactacattttagcgggccccattcagaattatagtataacctatctgtggtttgatttttatagtacccagcttagaaaacccgcaaaatacgtttactcacagtacaatcatacggctaaaacgataacattcagacccccaccttgtggtactgtgccttccatgacttgtctttccgaaatgctaaacgtttccaaacgtaatgatactggcgaacaaggttgcggtaatttcaccacgttcaaccccatgtttttcaatgtaccgcgttggaacaccaaattgtacgtgggtccgactaaggttaacgtagatagtcaaacgatttattttctaggtttaaccgccctgcttttacgttacgcacaacgcaactgtacacacagtttctacctggttaacgccatgagccggaatctatttcgcgtccccaagtatattaacggcaccaagttaaaaaacactatgcgaaaactaaaacgtaaacaagcgcccgttaaggaacaattcgaaaaaaaagctaagaaaactcagagtactactacgccatacttttcctatacaacgtctgccgctctcaacgtcactactaacgtgacttatagtattactaccgccgcaaggcgggtttccacgtctacaattgcttatcgtcctgatagcagctttatgaagtccattatggccacacagttaagggacctagcaacgtgggtgtataccactctacgttaccggcaaaatcctttttgtgaaccaagccgcaaccgaaccgccgtgtcagaatttatgaaaaacacgcacgtactaatccgtaacgaaacgccgtacactatttacggtactctcgacatgagctccttatattacaacgaaaccatgttcgtggaaaacaaaacagcttccgatagtaacaaaactacacctacgtcaccatcaatggggtttcagagaacatttatagatcccctgtgggactatctagactcgctgctgtttctagatgagattcgtaactttagcctccggtcacccacgtatgtaaaccttaccccgccggaacaccgccgggctgtaaatctgtccaccctcaatagcctttggtggtggttgcagtaa

SEQ ID NO: 34 - DNA, 1392 bp, Human herpesvirus 5 strain DM7
  AF531334.1 Human herpesvirus 5 strain DM7 UL74 gO1b glycoprotein gene
atggggagaaaaggagagatgagaggtgtttttaatttatttttccttatgtcgctaactttcctgctattctctttcataaactgtagggctgcggtaagattatccgtaggacgttactggtcaggtaaagtactgtctacgataggcaagcaaagactagacaagttcaaattagagattttgaaacaactagaaaaggatatctatacgaaatactttaatatgaccagacaacacattaaaaatctgactatgaacatgaccgagtttccacggtattacattttagcgggacccattcaaaataacagtgttacctatctttggttcgatttttatagcactcagcttcgaaaaccggctaagtacgttttctcagaatacaatcatacggctaaaacaataacattcagacccccatcttgcggtacggtgccttccatgacttgtctttccgaaatgctaaacgtttccaaacgtaatgatactggcgaacaaggctgcggcaatttcaccacgttcaaccccatgtttttcaatgtgccacgttggaacacgaagttatatgtaggttccaagaaagtaaacgtggatagtcaaacgatctactttttaggcctaaccgccttgcttctacgttacgcgcaacgcaactgtacgcacagtttctacctggttaacgccatgagccgaaatttatttcgcgtccctaagtatattaacggtactaagttgaaaaacactatgcgaaaactcaagcgtaaacaagctccagtcaaagaacaatcagaaaaaaagagtaagaaatctcaaagtactactacgccatattctccctatacaacgtcaaccgctctgaacgtcactactaacgcgacttatagtgttactaccaccacaaggcgggtttccacgtctacaattgcttatcgtcctgatagtagctttatgaagtccattatgaccacgcaattaagggacctagcgacgtgggtgtataccactctacgttaccggcaaaaccctttttgtgaatcaagccgcaaccgaaccgccgtatcagaatttatgaaaaacacgcacgtactaatccgtaatgaaacaccgtacactatttacggtactcttgatatgagctccttatattacaacgaaaccatgttcgtggaaaacaaaacggcttccgaaactacacctacgtcaccatcaacgggatttcagagaacgtttatagatcccctgtgggactatctagactcgctgctatttctagatgagatccgtaactttagtctccagtcacccacttatggaaaccttaccccgccggaacaccgtcgagctgtaaatctgtccacccttaatagcctttggtggtggttgcagtaa

SEQ ID NO: 35 - DNA, 1395 bp, Human herpesvirus 5 strain Toledo
  AF531355.1 Human herpesvirus 5 strain Toledo UL74 gO1c glycoprotein gene
atggggagaaaaggagacatgagaagcatttctaaattattctttattatatcactgactgtcctgttattttctataataaactgtaaggtcgtcagaccaccgggacgttactggttaggtacagtactttctacgataggcaagcaaaaactagataaattcaagttagagattttaaaacaattagaaagggaaccttatacaaaatacttcaatatgactagacaacacgttaaaaatcttactatgaatatgacccagtttccacaatactacattctagcaggtcccattcgaaacgatagtataacctatctgtggttcgatttttatagtacccagcttagaaaacccgccaaatacgtctactcacagtacaatcatacggctaaaacgataacattcagacccccatcttgtggtacagtgccttcaatgacttgtctttccgaaatgttaaacgtttccaaacgtaatgatactggcgaacaaggctgcggtaattttaccacgttcaaccccatgtttttcaatgtaccacgttggaataccaaattgtacgtgggtccgactaaagttaacgtagatagtcaaacgatttattttttgggtttaaccgccctacttctacgttacgcacaacgcaactgcacacacagtttctacctggttaacgccatgagccggaatctattccgcgtccccaagtatattaacggcaccaagttgaaaaacactatgcgaaaactaaaacgtaaacaagcgccagtcaaagaacaattagaaaaaaagactaaaaaatctcagagtactactacgccatatttttcctatacaacgtctaccgctctcaacgtcactactaacgcgacttataaggttaccaccagcgcaaagcggattcccacatctacgattgcttatcgtcctgatagcagctttatgaagtccattatggccacgcagttaagggatctagcgacatgggtgtatactactctacggtatcggaatgaacccttttgtaaaccagaccgtaaccgtaccgccgtgtcagaatttatgaaaaacacgcacgtattgattcgtaacgaaacaccgtacactatttacggcactcttgacatgagctccttatactacaacgaaaccatgtccgtggaaaacgaaacggcttccgataataacgaaactacacctacgtcaccatcgacgaggtttcagaaaacgtttatagatcccttatgggactatctagactcgctgctgtttctagataaaatccgtaactttagcctccaattacccgcgtatggaaatcttaccccgccggaacaccgccgggctgtaaatctatccaccctcaatagcctttggtggtggtcgcagtaa

SEQ ID NO: 36 - DNA, 1389 bp, Human herpesvirus 5 PH-BAC isolate
  AC146904.1119468-120856 Human Herpesvirus 5 PH-BAC isolate gO2a gene UL74
atgtggggaaaaggagagatgagaggtgttaacttattgtttcttatatggctaactttcctgttgttctttttcataaactgtaagggtgcgcgatcacaacgagcaccatttcgtcctaggatatggcaccccacggtattaaaaaaactaaagcaattaaaattagaaattttaagacaactagaacctattccttacataaagtacccacaaatcaatacgactcgagttcaaagtcttactgttaatatgaccgagtttccttattattatattttagcaggtcctattcgcaacgaaagtgtaactcatttgtggtatgatttctatagcacacagcttaggaaaccagccaaatatgtatactctatgtataatcacacggcccgaaaaataacgttcaaacccccgtcgtgtggcgcagtgccttccatgacttgtctttctgaaatgctaaacgtttccaagcgtaatgataccggcgaagaaggctgcggtaactttaccacgttcaatcctatgtttttcaatgtaccgcgttggaacaccaaattgtacgtgggtccgactaaagttaacgtagatagtcaaacgatttattttttaggtttaaccgccctgcttctacgttacgcgcaacgcaactgcacacacagtttctacctggttaacgccatgagccggaatttatttcgcgtccccaagtatattaacggcaccaagttgaaaaacactatgcgaaaactaaaacgtaaacaagcgcccgtcaaagaacaattagaaaaaaagaccaagaaatctcagagtactactacgccatatttgtcctatacaacgtctaccgctctcaacgtcaccactaacgtgacttatagtgttaccaccaccgcaaagcgggttttcacatctacgattgcttatcgtcccgatagcagctttatgaagtccattatggccacgcagttaagggatctagcgacatgggtgtatactactctgcgctatcgagatgaacctttttgtaaaccaaaccgtaacctgaccgccgtgtcagagtttatgaagaacacgcacgtattgatccgtaacgaaacaccgtacactatttatggtactcttgacatgagttccttatattgtaacgaaaccatgtccgtggataacgcgacggctttcgatagtaacaaaacgacacccacaccgttaccggggtttcagagaacgtttatagatcccctgtgggactatctagactcgctgctgttcctagataaaatccgtaactttagcctccagttacccgggtatggaaatcttaccccgccggaacaccgccgagctgtaaatctgtccacccttaatagcctttggtggtggttgcagtaa

SEQ ID NO: 37 - DNA, 1398 bp, Human herpesvirus 5 strain SW1715
  AF531342.1 Human herpesvirus 5 strain SW1715 UL74 gO2b gene
atggggaaaaaaaaaatactggtgagaggcgttcctaggatatttatggtatctacattcctgcttatttttctcataaactgtaaaggtgcgttaaacgtgccccgaggacgtccctggataggtaaagtaccctctttaaaatggaaacttaaagaacaacaattgaaaattgaaattttaaagcaattacagagcgacatatacacaaaatatccccagatcaccaaaaattacactcagtttattactacagagcttaaaaaatttccactgtactatatcctagcgggtcctattcgaaacgaaagtgtaactcacttgtggtttgatttttatagcactcaacttcgaaaacctgccaaatatgtttactctatgtataatcaaacggctcagaaaatcacgtttaggcccccgccttgcggtacagtgccttctatgaattgtttatccgagatgttaaatgtttccaagcgtaatgataccggcgaagaaggctgcggtaacttcaccacgttcaatcctatgtttttcaatgtaccgcgttggaacaccaaattgtacgtgggtccgactaaagttaacgtagatagtcaaacgatttattttctaggtttagccgccctgcttttacgttacgcgcaacgcaactgcacacgcagtttctacctggttaacgccatgagccggaatctatttcgcgtccccaagtatattaacggcaccaagttaaaaaacactatgcgaaaactaaaacgtaaacaagcgcccgttaaagaacaattcgaaaaaaagattaagaaatctcagagtagtactacgccatacttttcctatacaacgtctaccgctcccaacgtcactactaacgtgacttacggtgttacaaccaccgcaaggcgggttcccacatctacgatcgcttatcgttctgatagcagctttatgaagtccattatggccacgcagttaagagatctagcgacgtgggtgtatactactttgcgctatcgggatgagcctttttgcaaaccaaaccgtaaccggaccgccgtgtcagaatttatgaaaaacacgcatgtactgatccgtaacgaaacaccttacactatttacggtactctcaacatgagctccttatattacaacgaaaccatgtccgtggaaaacaaaacggcttccgatagtaacaaaactacacctgcgtcaccatcaacagtgtttcagagaacgtttatagaccccctgtgggactatctagactcgctgttatttataaatgaaatccgtaactttagcctccagtcatctgcgtatagaaaccttacccctccggaacaccgccgggctgtaaatctttccaccctcaataacctttggtggtggttgcagtaa

SEQ ID NO: 38 - DNA, 1395 bp, Human herpesvirus 5 strain SW475
  AF531348.1 Human herpesvirus 5 strain SW475 UL74 gO3 gene
atggggagaaaaggagagatgagaggtgtttttaacttattgtttcttatatcgttaacttttctgctattttctcttttgaactgtaagagcgccgcaagagttttcagactaccgtttccctatggcagggtattatcaaaaaatggcaggctagctgaaataaagtggaaacaagaactgttaaagcagataggtgcgagccaggattactataaattttttaccattcctactaaacaaggactaaacgctgttgtaacaatggaacggtttcccgataactatatcttggctggtcctattcaaaatgatagtattacctatatgtggttcgatttctatagtacccaactgcgaaaaccggccaagtacgtttactcagagtataatcaaacaggccgaaagatgaggtttagacctccatcttgtggcactgtgccatctatgggctgtctatctgagatgttgaacgtatcctttcgtaataacaccggagaagaaagctgtgttaatttgaccacgtttaatcctatgttttttaacgtaccacgttggaacactaaactgtacgttggttccactaaagtcaatgtggatagtcagacgatctactttttaggcctagccgccctgcttctacgttacgcgcaacgtaactgcacacgcagtttctacctggttaacgccatgagccgaaatctattccgcgttcccaagtatattaacggcaccaagttaaaaaacaccatgcgaaagcttaaacgtaaacaagcacccgtcaaagaaccgcaaaaaaagagtaagaaatctcaaagtactactacgccatctttctatacaacgtctacgactttcaacgtcaccaacgtgacttatagtgctaccaccactgcacggcggactcccacatctacgattgcttatcgtcctgatagcagctttatgaagtccattatgaccacacagttaagggacttagcgacgtgggtatacaccacgctacgctatcggcaagatcctttctgtagatcagatcgtaatcggaccgccgtgtcagagtttatgaaaaatacgtacgtattgattcgcaacgaaacaccgtacactatttacggcactcttgacatgagctccttatattgcaacgaaaccatgtccgtggaaaacgaaacggcttccgataataacgaaactacacctacgtcaccatcgacggggtttcaaagaacgtttatagatcccctgtgggattacctagactcgctgctgtttctaaataaaatccgtaactttagcctccagttacccgcgtatggaaaccttaccccgccggaacaccgccgggctgtaaatctgtctaccctcaatagcctttggtggtggttgcagtaa

SEQ ID NO: 39 - DNA, 1374 bp, Human herpesvirus 5 strain Towne
  AF531356.1 Human herpesvirus 5 strain Towne UL74 gO4 gene
atggggagaaagggagagatgagaggtgtttttaatttatttttccttatgtcgctaactttcctgctattctctttcataaactgtaagatcacggtagcgcgttttcgagtaaagagtcagaaagcaaaagaggaagagaggcaactaaaattacgtatactgcaagaactagcgtcaaaaacaggtgattattacaagtttttcacatttcctagtcagcaaaagttgtataacataactgtagaaatgaaacagtttcccccgaattccattttagcaggacctattcgtaatcatagcattacccacctctggttcgattttcacacgacccaactccgtaaaccggccaaatatgtgtattcggaatataatcatacagggcaaaaaataacatttcgacccccatcctgtggtacgataccttccatgacttgcctttctgaaatgctaaatgtatcccggcgtaacaacaccggggaggaaaactgtggcaattttaccacatttaatcctatgttttttaacgtaccacgttggaacaccaaactgtacgtgggtccgagtaaagtcaatgtggatagccaaacaatttattttttaggtttagccgccctgcttctacgttacgcgcaacgtaactgtacacgcagcttctacctggttaacgccatgagccggaatatattccgcgttcccaagtatattaacagcaccaagctgaagaacactatgcgaaagcttaaacgtaaacaagcgcctgtcaaatcgatcagtaagaaaagtcgtgttagcaccaccacaccatattcttcctacacgtcaactatttttaacgtcagtactaatgtaacttatagtcctattgtcccaactcggattcccacatctacaattggttatcgtcctgacgaaaactttatgaaatccattctgaccacgcagttaaaagatctagcgacgtgggtgtatactactctgcgctatcgagatgaacctttttgtaaaccaaaccgtaaccggaccgccgtatcagaatttatgaaaaatacgcacgtattgattcgcaacgaaacaccgtacactatttacggtactcttgacatgagctccttatattacaacgacaccatgcccgtggaaaacgaaacggcttccgataataacaaaactacacctacgtcaccatcgacgaggtttcagagaacgtttatagatcccatgtgggattatctagactcgctgctgtttttaagtgaaatccgtaacttcagcctacagtcgtccacatatggaaaccttaccccgccggaacaccgccgggctgtgaacctgtccaccctcaatagcctttggtggtggttgcagtaa

SEQ ID NO: 40 - DNA, 448 bp, Human herpesvirus 5 strain K141
  EU686518.114-461 Human herpesvirus 5 strain K141 glycoprotein O5a (UL74) gene, partial cds showing internal methionine initiation from gO5 genotype Merlin
atgataatggtgaaaggcattcctaaaattatgctcctgatttctataacgttcttgctcctttccctcataaattgtaatgtattggtaaactccagaggaacaagacgttcctggccgtataccgtgctatcttatcgaggtaaagagattctgaagaaacagaaggaagatatcttaaaacgattgatgtctacatcatctgacggataccgttttttaatgtaccccagtcagcaaaaatttcatgccatcgttattagcatggataaatttcctcaagactacattttagcgggtcccattagaaatgatagcattacccatatgtggtttgacttttacagtactcaactccgaaaaccagccaagtacgtatattccgaatataatcacacggcccacaaaataacgttacgacccccaccttgcggcacagtgccttcta

SEQ ID NO: 41 - DNA, 2493 bp, Human herpesvirus 6A strain U1102
  NC_001664.4c62129-59637 Human betaherpesvirus 6A, variant A DNA, virion genome, strain U1102 glycoprotein gB U39 gene
atgagcaagatggcagtattattcctggctgtctttttgatgaatagtgttttaatgatatattgtgatccggatcattatatcagagcgggctataatcacaaatatccttttcggatttgttcgattgccaaaggcacggatttaatgcggttcgacagagatatttcgtgctcgccgtataagtctaatgcaaagatgtcggagggttttttcatcatttacaaaacaaatatcgagacctacacttttccagtgagaacgtataaaaaagagttgacgttccaaagtagttaccgtgatgtgggtgtggtttattttctggatcggacggtgatgggtttggccatgccggtgtacgaagcaaatttagttaattctcatgcgcagtgttattccgccgtagcgatgaaacgacccgatggtacggtgtttagtgcctttcatgaggataataataaaaacaatactctaaatttatttcctctgaatttcaaatctataactaataaaagatttatcactacgaaagaaccctactttgcaaggggtcctttgtggctatattctacatcaacgtctctcaattgtattgtgacggaggctacggctaaggcgaaatacccgtttagttactttgctttgacgactggcgaaatcgtggaagggtctccgttcttcaacggttcaaacggtaaacattttgcagagccgttagaaaaattgacaatcttggaaaactatactatgatagaagatctaatgaatggtatgaatggggccactacgttagtgaggaaaatcgcttttctcgagaaagcggatactttgttttcttgggaaatcaaggaagagaatgaatcggtgtgtatgctaaagcactggaccacggtgactcacgggcttcgagcggagacgaatgagacctatcactttatctctaaggagttgacagccgctttcgtcgcccccaaggagtccttaaatcttaccgatccgaaacaaacgtgtattaagaatgaatttgaaaaaataattaatgaagtctatatgtcagattataatgatacatatagcatgaatggtagttatcaaatttttaagactacgggagatttgattttgatttggcaacctcttgtgcaaaaatctcttatgtttcttgagcagggttcggaaaaaatacgtaggaggcgagatgtgggggatgttaagtctagacatgatattctttatgtgcaattacagtatctttatgatactttgaaagattatatcaatgatgcattggggaatttggcagaatcttggtgtctcgatcaaaagcgaacgataacgatgttgcacgaacttagtaagattagtccgtcgagtatcgtgtctgaggtttacggtcgtccgatatctgcacagttgcatggtgacgtgttagctatctcgaaatgcatagaggttaatcagtcatccgttcagcttcataagagtatgcgggtcgtcgatgcaaagggagtaaggagtgaaacgatgtgttataatcggcccttggtgacgtttagttttgtgaactcgacgcctgaggttgtccctggccagctagggttagataatgaaattctgttgggtgatcataggacagaggaatgtgaaatacctagtacaaagatctttttatctggaaatcatgcgcacgtgtataccgattatacgcatacgaattcgacgcccatagaagacattgaggtattggatgcttttattagactaaaaatcgatcctcttgaaaatgccgattttaaagtactcgatttatattcgccggacgaattgagtagagcaaacgttttcgatctagagaatattcttcgtgaatataactcatataagagcgcactatatactatagaagctaaaattgctacgaatacaccttcgtatgttaatgggattaattcttttttacaagggcttggggctataggcactggattgggctcggttataagtgttacggcaggagcgcttggggatattgtgggtggagtggtgtcttttctaaaaaatccattcgggggtggtctcatgttgattttagcgatagtagttgttgtgataataattgtggttttcgttagacaaagacatgtgcttagtaagcctattgacatgatgtttccttatgccaccaatccggtgactactgtgtccagtgttacggggaccactgtcgtcaagacacctagtgttaaggatgttgacgggggcacatctgttgcggtttcggaaaaggaggagggtatggctgacgtcagtgggcaagtaagtgacgatgaatattcacaagaagatgctttaaaaatgctcaaggccataaagtccttagacgagtcctacagaagaaaaccttcgtcttctgagtctcatgcctcaaaacctagtttgatagacaggatcaggtatagaggttataagagtgtaaatgtagaagaagcgtga

SEQ ID NO: 42 - DNA, 2085 bp, Human herpesvirus 6A strain U1102
  NC_001664.4c80170-78086 Human betaherpesvirus 6A virion genome, strain U1102, glycoprotein gH U48
atgctcctccgactctgggtctttgtcctgttgactccctgttacggttggagaccgttgaacatatcgaactcgagccattgtagaaatggaaactttgaaaatccaatcgttcgccccggctttataacatttaacttttatacaaaaaacgacactcggatatatcaagtccctaaatgcttactcggctccgatatcacataccacctgtttgatgccatcaacacgacagaatcgttaacaaattatgaaaaacgcgttacacgtttttatgagccgccaatgaacgatattttaagactttctcctgtaccttcggtcaaacaatttaacttagatcgctctatccaaccgcaggttgtgtattccttaaacatgtacccttcacagggaatttattacgtcagggtcgtagaagttcgacagatgcaatacgacaacgtttcctgtaagctgcctaattctctcaaggaactaatatttccagtccaagtcagatgcgctaaaattacgcgctatgtgggcgaagacatctatacccatttctttactccggactttatgatactgtacatccagaatcccgcgggagatctgactatgatgtacggaaacaccaccagcataaactttaaagccccttataagaaaagttcattcatattcaaacagacattgacagacgatttactattgatagtcgaaaaagacgtaatagatgtacaataccgtttcatatcagatgcgacattcgtagacgaaacgttgaatgacgtagatgaagtagaagctctactactcaaatttaataacctaggaatccaaaccctattaagaggagactgtaaaaaacccaactatgccggcataccgcagatgatgtttctttacggtatcgtacatttctcatatagcacaaaaaacacaggaccaatgcccgtgttaagagtgttaaagacacacgaaaatctcctgtccatcgactcatttgtcaaccgatgtgtgaacgtatcggaaggtacgttacaatacccaaaaatgaaggaatttttaaaatacgagccctcggactatagctacataaccaaaaacaaatccatttccgtatctacgctgctcacgtacttagcgacagcgtacgaatccaatgtaacgatttccaagtacaagtggactgacattgccaacactctacaaaacatctatgaaaaacacatgttttttactaatctgacattttccgatagggaaactctattcatgctagccgaaatagcgaatatcatccctaccgatgaacgcatgcagaggcacatgcaactactaattggaaacctgtgtaaccccgtagaaatagtttcatgggcccgcatgcttacagctgacagggcaccgaatctagaaaatatttattcgccttgtgcctcccccgtacgcagagatgtgacaaattcttttctaaaaacagttctcacgtacgcttcccttgaccgttatcgatcagacatgatggagatgctatccgtatacagaccgccaaatatggagagagtagcggctattcagtgtctctccccaagtgaaccagcagcttctctgactctgccgaatgtgacatttgtaatttctccctcttacgtgattaaaggagtgagcctaacaattacaacgacaattgtggctacgagtataataatcacagccatacctctcaattccacctgcgtttcaaccaactataaatatgcaggacaagatctgcttgtgctacgaaacatctcatctcaaacatgcgagttctgtcagagcgtagtcatggaatatgatgatatcgacggtcccttacaatacatttacataaaaaacatagacgaactaaaaacattgacagatcccaacaacaatttacttgttcccaacaccaggacgcactatcttttgttagccaaaaacggctctgtttttgagatgtctgaagtcggaatcgatatagaccaagtgtctatcatattggttatcatttatattctgatcgcaataattgctttattcggattatatagacttatcagattgtgttga

SEQ ID NO: 43 - DNA, 753 bp, Human herpesvirus 6A strain U1102
  NC_001664.4c123457-122705 Human betaherpesvirus 6A virion genome, strain U1102 glycoprotein gL U82 gene
atggaacttttactatttgtaatgtcactcatattactaaccttctcaaaagcaatacctcttttcaaccataattctttctattttgaaaaacttgacgactgtatcgcagctgtaataaactgcacgaaatccgaagtgcctctattactagaaccaatctaccagcctccggcatataatgaagacgttatgtcaatactgctacaacccccgacaaaaaaaaagccttttagccgtataatggtaaccgatgaatttctcagcgacttcttactcctacaggataacccagaacaactacgcacattgtttgcgctaataagggacccagaatctcgggacaactggctaaactttttcaatggcttccagacatgttcaccttccgtcggaataacaacctgcatcagagataactgtagaaaatattcgcccgaaaaaattacgtacgtcaataacttttttgttgataacattgcgggtctcgagtttaacatttcagaaaacacagacagtttttacagcaacattggttttttattatacttggagaatcctgctaaaggcgtcacaaaaattattaggttcccttttaactctttgactctttttgatacgattttgaattgtttaaagtatttccacttgaaaaccggagtagagctcgacctgctaaaacatatggaaacctacaattctaaactacctttccgaagttcccgccctacaattctgattagaaacacataa

SEQ ID NO: 44 - DNA, 1956 bp, Human herpesvirus 6A strain U1102
  NC_001664.4c77919-75964 Human betaherpesvirus 6A virion genome, strain U1102 gO U47 gene
atgcatttggaggtcatcgtccaatcctataaaaaaagcaaatactattttagtcacacattctacttgtacaaattcattgtggtcaactcacccgacatgcttcacatctcgcgactcggcctttttctgggccttttcgcgatagtcatgcactccgttaatctaataaaatacacgtctgatcccttagaagctttcaaaaccgtcaaccgccacaattggagcgacgaacaaagagaacatttttacgacctccgaaacttatatacaagtttttgtcagacaaatctatccctcgactgcttcactcaaattttaactaacgttttctcttgggacattcgagattcacaatgcaagtctgcggttagcttgtctccattacagaatttaccgcggacagaaatcaaaatagtgctatcctcgacgactgcaaacaaatctatcatcgctagcagtttttctctattttatcttctgtttgccacactatctacatataccgcagatccgccatgcgtagagttactaccatttaaaattctgggagcacagttatttgacataaaactaaccgaagaatccttgcggatggcaatgagcaaattttccaactcgaatctgacacggtcattgacttccttcacgtcaaaaaacttctttaattacaccagctttgtttacttcttgctctataacacaacatcatgcgtcccttcaaatgatcaatatttcaaacagtcgccaaaacctataaatgttaccacttcctttggacgagccatcgtaaactttgattcgatactaactactacaccatcatcgacgtcagcgtctctcacatcaccacatatccctagtaccaacataccaaccccagcacctccccccgtaacaaaaaactctacaaaactgcatacagacaccataaaagttacaccgaacacacccactataacaacgcaaacaacggaaagcatcaaaaaaatagttaaacgttcagattttcctcgacccatgtacaccccaaccgacattccaactcttacaatccgtcttaacgccactattaaaaccgaacaaaacaccgaaaacccaaaaagtccaccaaaaccaacaaattttgaaaataccacaatcagaattcccaaaacccttgagagcgctacagcaacaacaaacgcaacccaaaagatcgaaagcaccaccttcacaacaataggaatcaaggaaattaacggcaatacctattcttcaccaaaaaactctatttatcttaagagcaaatcacagcagagtacaacaaaattcaccgacgccgaacacaccactccgattttaaagtttaccacttggcaaaacacggtacgcacatacatgagtcacaacacagaagtacaaaacatgaccgacaaattccagaggacaaccttgaaatcctcaaacgagctacctaccattcagacgttgtctgtcactccaaaacaaaaactaccgtcgaatgtaactgccaaaactgaagtacacataactaacaacgctttaccatctagtaattcatcatactcaatcactgaagtcactaaagaggtaaagcatactagaatgtcagcgtccactcacgaacagataaaccacacagaaatagcacaaataacaccaattcttaacgctcacacatcggaaaaatcaactacacctcaacggtccttcaccgctgaaacgttcttaacgacatcttcaaagcctaacatcataacctggtcaaacttactaacaacaacgcctaaggaaccattaacgaatacaagtctaaggtggacagatcatatcacaacacagctaacgactagcaatagaactcaatcagccaaactaacaaaagctaacatctcgtcacaaacgactaacatctacccccaaacaatcacgggacgatctacagaggtttaa

SEQ ID NO: 45 - DNA, 2493 bp, Human herpesvirus 6B strain Z29
  NC_000898.1c63200-60708 Human herpesvirus 6B, strain Z29 genome gB U39 gene
atgagcaagatgagagtattattcctggctgtctttttgatgaatagtgttttaatgatatattgcgattcggatgattatatcagagcgggctataatcacaaatatccttttcggatttgttcgattgccaaaggcactgatttgatgcggttcgacagagatatttcgtgttcgccatataagtctaatgcaaagatgtcggagggttttttcatcatttacaaaacaaatatcgagacctacacttttccagtgagaacatataaaaacgagctgacgttcccaaccagttaccgtgatgtgggtgtggtttattttctggatcggacggtgatgggtttggccatgccggtgtacgaagcaaatttagttaattctcgtgcgcagtgttattcagccgtagcgataaaacgacccgatggtacggtgtttagtgcctatcatgaggataataataaaaacgaaactctagaattatttcctctgaatttcaagtctgttactaataaaagatttatcactacgaaagaaccctactttgcaaggggtcctttgtggctctattctacatcgacgtctctcaattgtattgtgacggaggctacggctaaggcgaaatatccgtttagttactttgctttgacgactggtgaaatcgtggaagggtctccgttcttcgacggttcaaacggtaaacattttgcagagccgttagaaaaattgacaatcttggaaaactatactatgatagaagatctaatgaatggtatgaatggggctactacgttagtaaggaagatcgcttttctggagaaaggggatactttgttttcttgggaaatcaaggaagagaatgaatcggtgtgtatgctaaagcactggactacggtgactcacgggcttcgagcggagacggatgagacttatcactttatttctaaggagttgacagccgctttcgtcgcctccaaggagtctttaaatcttaccgatcccaaacaaacgtgtattaagaatgaatttgagaagataattacagatgtctatatgtcagattataatgatgcatacagcatgaacggtagttatcaaatttttaagactacgggagatctgattttgatttggcagcctcttgtgcaaaaatctcttatggttcttgagcagggttcagtaaacttacgtaggaggcgagatttggtggatgtcaagtctagacatgatattctttatgtgcaattacagtacctctatgatactttgaaagattatatcaacgatgccttggggaatttggcagaatcttggtgcctcgatcaaaaacgaacgataacgatgttgcacgaacttagtaagatcagtccatcgagtatcgtgtctgaggtttacggtcgtccgatatctgcacagttgcatggtgatgtgttagctatctcgaaatgcatagaagttaatcaatcatccgttcagctttataagagtatgcgggtcgtcgatgcgaagggagtaaggagtgaaacgatgtgttataatcggcccttggtgacgtttagctttgtgaactccacgcctgaggttgtccttggtcagctagggttagataatgagattctgttgggtgatcataggacagaggaatgtgagatacctagtacaaagatatttctatctggaaatcatgcacacgtgtataccgattatacgcatacgaattcgacgcccatagaagacattgaggtattggatgcttttattagactaaagatcgaccctctcgaaaatgctgattttaaactacttgatttatattcgccggacgaattgagtagagcaaacgttttcgatttagagaatattcttcgtgaatataactcatataagagcgcactatatactatagaagctaaaattgctactaatacgccgtcgtatgtcaatgggattaattcttttttacaagggcttggggctataggcactggattgggctcggttataagtgttacggcaggagcacttggggatattgtgggtggagtggtgtcttttttaaaaaatccattcgggggtggtctcatgttgattttagcgatagtagttgtcgttataataattgtggttttcgttagacaaaaacatgtgcttagtaagcctattgacatgatgtttccttatgccaccaatccggtgactactgtgtccagtgttacggggaccactgtcgtcaagacgcctagtgttaaagatgctgacgggggcacatctgttgcggtttcggaaaaagaggagggtatggctgacgtcagtggacaaataagtggtgatgaatattcacaagaagatgctttaaaaatgctcaaggccataaagtctttagacgagtcctacagaagaaaaccttcgtcttctgagtctcatgcctcaaaacctagtttgatagacaggatcaggtatagaggttataagagtgtaaatgtagaagaagcgtga

SEQ ID NO: 46 - DNA, 2085 bp, Human herpesvirus 6B strain Z29
  NC_000898.1c81349-79265 Human herpesvirus 6B, strain Z29 genome gH U48 gene
atgctcttccgactctgggtctttgttctgttgactccctgttacagttggagaccgtggaccatatcggacgagagccattgtaaaaatggaaactctgaaaatccaattgttcgcccgggctttataacttttaacttttatacaaaaaacgacactcggatatatcaagtccctaaatgcttactcggatccgatatcacgtaccatctgtttgatgccatcaacacgacagaatcgttaaccaattatgaaaaacgcgtcacacgtttctatgagccgccaatgaacgatattttaagactttctaccgtacctgcggtcaagcaatttaacctggatcactctatccaaccgcagattgtctattccttaaacctgtacccttcacacggaatttattacatcagggttgtggaagtccgacagatgcaatacgacaacgtttcctgtaagctgcctaattctctcaacgaactgatctttccagtccaagttagatgcgctaaaattacacgctatgcgggcgaaaacatctatacccatttctttactccggactttatgatactgtacatccaaaatcccgcaggagatctgactatgatgtatggaaataccaccgacataaactttaaagccccttataggaaaagttcattcatattcaaacagacattgacagacgatctactattgatagtcgaaaaagacgtagtcgatgaagaataccgtttcatatcagatgcgacattcgtagacgaaacgttggatgacgtagatgaagtagaagctctactactcaaatttaataacctaggaatccaaaccctattaaggggagactgtaaaaaacccgactatgccggcataccgcagatgatgtttctttacggtatcgtacatttctcatatagcacaaaaaacacaggaccgatgcccgtgttaagagtgttaaagacacacgaaaatctcttgtccatcgactcatttgtcaaccgatgtgtgaacgtctcggaaggtacgatacaatacccaaaaatgaaggaatttttaaaatacgagccctcggactatagctacatcaccaaaaacaaatccattcccgtatctacgctgctcacgtacttagcgacagcgtacgaaaccaatgtaacgatttccaggtacaagtggtctgacattgccaacactctacaaaagatctatgaaaaacacatgttttttacgaatctgacattttccgatagggaaactctattcatgctagccgaaatagcgaatttcatccctgccgatgaacgcatgcagaggcacatgcaactactaattggaaacctgtgtaaccccgtagaaatagtttcatgggcccacatgcttacagctgacaaggcaccgaatctagaaaatatttattcgccttgtgcctctcccgtacgcagagatgtaacaaattcttttgtaaaaacagttctcacgtacgcttccctcgaccgttatcgatcagacatgatggagatgctatccgtatacagaccgccagatatggcgagagtagcggccattcagtgtctctctccaagtgagccagcagcttctctgcctctgccgaatgtgacatttgtaatttctccctcttatgtgattaaaggagtgagtttaacaattacaacgacaatcgtggctacgagtataataatcacagccatacctctcaattctacctgcgtttcaacaaattataaatatgcaggacaagatctgcttgtgctacgaaacatatcatctcaaacatgcgagttttgtcagagcgtagtcatggaatatgatgatatcgacggtcccttacaatacatctacataaaaaacatagacgaactaaaaacactgaccgatcccaacaacaatttacttgttcccaacaccaggacgcactaccttttgttagccaaaaacggctctgtttttgagatgtctgaagtcggaatcgatatagaccaagtatctatcatattggttatcatttatgttctgatcgcaataattgctttattcggcttatatagactcatcagattgtgttga

SEQ ID NO: 47 - DNA, 753 bp, Human herpesvirus 6B strain Z29
  NC_000898.1c124745-123993 Human herpesvirus 6B, strain Z29 gL U82 gene
atggaacttttactatttgtaatgtcactcatattactaaccttctcgaaagcgatgcctcttttcgaccacaattctttctattttgaaaaacttgacgactgtatcgcagctgtaataaattgcacgagatccgaagtacctctattactagaaccaatctaccagcctccggtatataatgaagacgttatgtcaatactgctaaaacccccaacaaaaaaaaagccttttagccgtataatggtaaccaatgaattcctcagcgacttcttacttctgcaggataacccagagcaactacgcacattgtttgcgctgataggggacccagaatctcgggacaattggctaaactttttcaatggcttccagacatgttcgccttccgtcggaataacaacctgcatcagcgataactgtagaaaatatttgcctgaaagaattacgtacgtcaataacttttttgttgataacattgcaggtctcgagtttaacatttcagaaaacacagacagtttttacagcaacattggttttttattatacttggagaatcctgctacaggcatcacaaaaattatcaggttcccttttaactctttgactctctttgatacgattttgaattgtttaaagtatttccacttgaaaaccggagtagaattcgacctgctaaaacagatggaagcctacaattctaaactacctttccgaagttcccgccctacgattctgattagaaacacataa

SEQ ID NO: 48 - DNA, 2217 bp, Human herpesvirus 6B strain Z29
  NC_000898.1c78999-76783 Human herpesvirus 6B, strain Z29 glycoprotein gO U47 gene
atgcttcacatctcgcgactcggcctttttctggcccttttcgcgatagtcatgcactccgttaatctaataaaatacacatctgatcccttagaagctttcaaaaccgtcaaccgccacaattggagcgacgaacaaagagaacatttttacgacctccgaaacttgtatacaactttttgtcagagaaatctatccctcgactgcttcactcaaattttaactaacgttttctcttggaacattcgagatttacaatgcaagtctgcggttaacttgtctccattacagaatttaccgcgggcagaaaccaaaatagtgctatcctcaacagctgcaaacaaatctatcgtcgctagcagtttttctctattttatcttctgtttgccacactatctacatataccgcagatccaccatgcgtagagctactaccgtttaaaattctgggaacacagttatttgacataaaactgaccgacgaatccttgcagatggcaatcagtaaattttccaactcgaatctgacacggtcattgactccgttcactcctgaaatcttctttaattacaccagctttgtttacttcttgctctataacacaacatcatgcatccgttcaaatgatcaatatttcgaacattcgccaaaacctataaatgttaccacttcctttggacgagccatcgtaaactttcattcgatactgactacgacgccatcatcgacgccatcatcgacgtcagcgtctatcacatcaccacatatccctagtaccaacacaccaaccccagaaccttcccccgtaacaaaaaactttacagaattgcagacagacaccataaaagttacaccgaacacacccaccataacagcgcaaacaacggaaagcatcaaaaaagtagttaaacgttcagattttcctcgaccgatgtacaccccgactgacattccaactcttacaatccgtcgtaacgccactattaaaaccgaacaaaacaccgaaaaccctaccgaaaacccaaaaagtccaccaaaaccaacaaattttgaaaataccacaatcagaattcccgaaacgtttgagagcactacagtggcaacaaacacgactcaaaagctcgaaagcaccaccttcgcaacaacaataggaatcgaggaaattagcgacaatatctattcttcaccaaaaaactctatttatcttaagagcaaatcacagcagagcacgacaaaattcaccgacaccgaacacaccactccgattttaaagtttaccacttggcaagacgcggcgcgcacatacatgagccacaacacggaagtacaaaacatgaccgaaaattttattaaaatttctcttggagaaacgatgggaatcacgcctaaagaacccacaaatcctacccaacttctcaacgtaaaaaaccaaacagaatacgcaaacgaaacccatagcacagaagtgcagaccgtcaaaaccttcaaagaggacagattccagaggacaactttgaaatcctcaagcgagccacctaccgttcagacgttgtctgtcactccaaaaaaaaaactaccgtcaaatgtaactgccaaaactgaagtacaggtaactaacaatgctttaccatctagtaattcatcacactcaatcactaaagtcaccgaagaaccaaagcaaaatagaatgtcggcgtccactcacggagagatcaaccacacagaaataccacgaatgacaccaattcttaacgctcacacatgggaaaaatcgactacacctcaatggcccttcaccgctgaaacgtccttaacgacatcttcaaagtctgccattctaacctggtcaaacttactaacaacgccaaaggaaccattaacgaatacaagtctaaggtcgacaaatcatatcacaacacagctaacgactagcaacagaactcaatcagccaaactaacaaaagctcacgtctcgtcacaaacgactaacatctacccccaaacaatcacggaacgatctacggacgttaaaaaaaaaagttccacagaaagtcgggaagctaacaaaactctgcctggaaacgactaccgcgtcaccgataaaaactcacacaaccatccagataacctcactaccaaagcatactcaacccaaaatgcaacgcattacacatacaacgaacgacacgatttaaacaacacagatagcacataa

SEQ ID NO: 49 - DNA, 2469 bp, Human herpesvirus 7
  NC_001716.2c61093-58625 Human herpesvirus 7 genome, glycoprotein gB U39
atgaaaattctattcctgagtgtttttataacttttagcttacagctatctctacaaacagaagctgactttgtcatgactggacacaatcagcatttaccatttcgaatttgttcaattgccaccgggacagatttagtgcgttttgacagggaggtttcttgcgcgtcttacggctctaacattaaaactaccgaaggtattttgataatttacaaaacaaagattgaagcacacacgttttctgtcagaacttttaaaaaagaacttacatttcaaacaacatatcgcgatgttggcactgtgtatttcttagatcgaactgttacaactttgccaatgccaattgaagaagtgcacatggtaaacaccgaggcgcgttgtttgtcctctatctctgtaaaacgttcggaggaagaggagtatgttgcatatcataaagatgaatatgtgaataaaacgttggatttgattccgttaaattttaaatctgatactgtcagaagatatataactactaaagaaccatttttaagaaatggtcccctctggttctattcaacatctacatcgataaattgcatagttacagactgcattgcaaagactaaatatccttttgatttttttgctttatcaacaggggaaaccgtggaagggtcaccgttttataatggtattaattctaaaacatttaatgaaccaacggaaaaaattttgtttagaaataattatactatgctgaaaacgtttgatgatggatcaaaaggaaattttgtgacgttaactaaaatggcttttctggaaaagggaaatactattttttcatgggaagtgcagaatgaagagtcttccatttgtttgttaaagcattggatgacaatcccacacgctttacgggcagaaaatgctaacagttttcactttattgcgcaggaactaactgcttcttttgtcacaggaaaaagtaattatacgctttctgattcaaaatataattgtattaacagcaattatacttcaattttggatgagatttaccaaacgcaatacaacaattcgcatgacaaaaatggtagttatgaaatttttaaaactgagggagatttaattctgatttggcaaccgttaatacaacggaaattaacggttttggaaaatttttctaatgcttctagaaaaaggaggaagagggaattagagactaacaaagacatcgtatatgttcaactacaatacctatacgacactctgaaagattacattaacacagcactaggtaagcttgctgaagcctggtgtttagatcaaaaacgcactatcacagtgttgcacgagcttagtaaaataagtccttctggaatcatttcagcagtgtacggtaaacccatgtccgctaaattaattggtgatgtattagcagtctcaaagtgcattgaagtcaaccagacttctgttcagctacataaaagtatgagattgacaaaagattcaagttacgatgctctaagatgttattctcggccattgttaacatattcatttgcaaattcttcgaaggaaacttatttaggacaacttggtttagacaatgagatattactgggaaatcacagaacagaagaatgtgaacaatctaacacgaaaatttttttgtctggtaaattcgctcatatttttaaagactatacgtacgttaattctagtttgataacagaaatagaagctttagatgcgtttgttgacttaaacatagatcctttagaaaatgcagattttacattattggaattgtatacaaaagatgaactgagcaaagcgaacgtttttgatttggaaaccattcttagagaatacaattcttacaaaagtgcgctacaccatatagaaacaaaaattgcaactgttactccaacatatataggagggattgacacatttttcaaaggtcttggcgctcttggtcttggtttgggggctgtgctgggtgtaacggctggtgctttgggagatgtggtgaatggagttttctcttttcttaaaaatccatttggtggagcactaactatcttactaactttgggagtgattggcttagttattttcttatttttaagacataaacgattagcacaaacaccgattgatattttatttccttatacatcaaaatcaacaaattcggtacttcaagcaacgcaatcagttcaagcgcaagtgaaagaacctttagattcatctccgccttatttaaaaactaacaaagacacagaaccgcaaggtgatgacataacacacactaatgaatattcacaagtggaagctttaaaaatgttgaaagctattaaattattagatgagtcatataaaaaagcggaaatcgctgaggcaaaaaaatcacagagaccaagcctacttgaaaggattcaatacaggggctatcagaaactttcaacagaagaactgtga

SEQ ID NO: 50 - DNA, 2073 bp, Human herpesvirus 7
  NC_001716.2c77113-75041 Human herpesvirus 7genome glycoprotein gH U48 gene
atgtatttttacataaatagtctacttctcatcgtgtcaataaatggatggaaacactggaatatactcaattcttcaatttgcgtaaatgaaaaaacaaatcaaactattattcaacctggtttaataacgtttaatttccatgattataacgaaactcgggtttatcagattcccaaatgcttattcggatatacttttgtctcgaatttatttgattctgtgaatttcgatgaatcatttgatcaatataaacatcggattacacgtttttttaacccatcaacagagaaagctgtgaaaatctatgcacaaaaatttcagacaaatatcaaacctgtttcgcatacaaaaacgattacagtgtctttattacctttattttatgaaaaggatgtctactttgcaaatgtttcggaaattcgcaaactctattacaaccaatacatttgcacactttcaaatggcttgactgattatctattcccaataacagaaagatgtgttatgagacattataattacttaaatactgtgttcatgctagctttgacaccatcgttttttataatttcagtggaaacaggaatggatgatgtcgtttttatctttggaaatgttagtcgtattttttttaaagcaccttttcgaaaaagttcattcatatatagacagactgtctcagacgatctcctgttaattacaaaaaaaacaacaattgaaagattttatccgtttctcaaaatagactttctggatgatatttggaaacaaaattatgacatctcgtttttaatagccaaattcaataagttagcaactgtttatatcatggagggattttgcggaaaaccagttaacaaagacacattccatttaatgtttttattcggcttaactcattttctatatagtactcgaggagatggactcctccctctgttggaaatccttaatactcaccaaagcatcattacgatgggtaggtttttagaaaagtgcttcaaaatgactaagagtcatttattatatcctgagatggaaaaacttcagaatttccagttagtagattattcttatattacatctgatctgacaattcctatctctgctaagctagcatttttatctttagcagatggaagaatcgttacagttccacaaaacaaatggaaagaaatagaaaacaatattgaaacattatatgaaaaacataaattgtttacaaatttaacccaaccggaacgtgcaaatttgtttttactctcagaaattggaaacagtctagtcttccaagaaaagatcaagcgaaagattcatgttttgctcgcaagtctgtgcaatcctttggaaatgtatttttggacacatatgcttgataatgtcatggacattgaaacgatgttttctccttgtgcaacagccacgcgcaaagatttaactcaaagagttgtcaacaacattttatcctataaaaacctagacgcatacacaaacaaggtgatgaacacactttctgtatatagaaaaaagagattagatatgtttaaaagcatttcctgtgtttcaaatgaacaagctgccttcctcacattaccaaatattacttacaccatctcttcaaaatatattttagccggtactagtttcagtgtcacgagtactgtcataagcactacaattattatcactgttgttcccttaaactctacctgcacacctactaattataaatattcggtgaaaaatataaaaccaatctataatatttcatcccacgattgtgtgttttgtgaaagcttggttgtggaatatgacgacatagacggaattatccaatttgtatacataatggatgacaagcaactattaaaattaatagatccggacacaaattttatcgatgtgaatccacgaacacattatttattgtttttaagaaatggctctgttttcgaaattaccgcattagatctaaaatcaagtcaagtttcaatcatgttagttcttctgtatttaattatcataattatagttttgtttggtatttatcatgtatttagattgttttga

SEQ ID NO: 51 - DNA, 741 bp, Human herpesvirus 7
  NC_001716.2c121009-120269 Human herpesvirus 7genome, glycoprotein gL U82
atgaaaactaacatcttttttatatttcttatctccattctaaaccaaatatatgctttatttaataactcttattattctaatttagaacaagaatgtatcaaaaacattctaaattgtacccaatcaaaaactctgtcgcttctggaacctattgatcaggcacctataccaaattcggatataatttctcgcttattatatcatactccctatatctcaaggagagatcaagttttaattgatgaagattttttagaaacattttacctactgtataataatcctaatcaactacacaccttactttccttaattaaagattcagaatcagggcacaactggcttggctttttaaataactttgaaagatgtctttcagataacacacttttaacctgtagagataatgtgtgtaaatcatactcttatgaaaaactcaaattcacgggcaacatctttgtagaaaatataatcggatttgaattcaatataccatcaaatatgataaattttaacatgtcaattttgatttaccttgaaaatgaagagactcgcacacagagaattgtgagaattgatcaccatggaattaatgtttttgatgctttgttaaattgtttgagatatttttctaggtattacaactttagtttcccgttaattcaagaaatggaaaaatataatgaagtgcttccattccgaagcgagttttcgaatttgctcattagaacttactga

SEQ ID NO: 52 - DNA, 942 bp, Human herpesvirus 7
  NC_001716.2c74803-73862 Human herpesvirus 7genome, glycoprotein gO U47 gene
atgaaaaacaaaatgtattcaatgctagtgttcacgttgatttcttttcttttttatagttatctgatcatatggactcctgtattaagtgtttgctctgaaaaaatctcgtttgtaaatttaacgaatttttccatgtggccgaaattcgcaaaatttcattatgagtcttttgaaaaaacatatattcagagttgcgacatacccatctctaagaactgttttaaacagattttattttggtcattccgtctatcccagaaaaagaatatttgcctttctaaaataaatatgctctatctagataattttccgcgatggacattacattttgaatttcccactaaaacaaacagaaaacgtcacgtttacttagataatatttcctttttgtatttagtttttgctacactagcattcaaaacgatggatgactcttgtgtcaataaaataccgtttgaagtacttgcttcacatttgtttaaaattaactttagccaaaacaagattgaaaccattttccaagatctccaaaaccatactgaagctcaattattttcacgcgaaaattctgagcaacttttttcttttacgaattttatttactactttgtttacaatcgaacagattgcaagaattctatttcaaaatttttctataattctgtgaacacgcggaatgtgtcaacaccatttggtgttaccaacttctccttggtaaggggaatcatgtcaccaatacaaagttttaaggggaacttgatgttcttagaaaataagaccaaagttactaagcctacagcaattccgaacgctgttacttccgaaccaacaaaattttttccatctactcgaggcacctcaagcatgcaagcttcacaacagacttcatcatttcctacgacctttttcacgacaaaccatagtaacacaagcacataa

SEQ ID NO: 53 - DNA, 689 bp, Human herpesvirus 8 strain GK18
  NC_009333.176014-76437,76532-76794 Human herpesvirus 8 strain GK18genome glycoprotein K8.1 with transmembrane domain
atgagttccacacagattcgcacagaaatccctgtggcgctcctaatcctatgcctttgtctggtggcgtgccatgccaattgtcccacgtatcgttcgcatttgggattctggcaagagggttggagtggacaggtttatcaggactggctaggcaggatgaactgttcctacgagaatatgacggccctagaggccgtctccctaaacgggaccagactagcagctggatctccgtcgagtgagtatccaaatgtctccgtatctgttgaagatacgtctgcctctgggtctggagaagatgcaatagatgaatcggggtcgggggaggaagagcgtcccgtgacctcccacgtgacttttatgacacaaagcgtccaggccaccacagaactgaccgatgccttaatatcagccttttcaggatcatattcatctggggaaccatccaggaccacgcgaattcgcgtatcaccggtcgcagaaaacggcagaaatagtggtgctagtaaccgtgtgccattttctgccaccactacaacgactagaggaagagacgcgcactacaatgcagaaatacggacccatctttacatactatgggctgtgggtttattgctgggacttgtccttatactttacctgtgcgttccacgatgccggcgtaagaaaccctacatagtgtaaca

SEQ ID NO: 54 - DNA, 504 bp, Human herpesvirus 8
  AF068830.11-504 Kaposi′s sarcoma-associated herpesvirus glycoprotein K8.1B mRNA, complete cds BCBL1 strain
atgagttccacacagattcgcacagaaatccctgtggcgctcctaatcctatgcctttgtctggtggcgtgccatgccaattgtcccacgtatcgttcgcatttgggattctggcaagagggttggagtggacaggtttatcaggactggctaggcaggatgaactgttcctacgagaatatgacggccctagaggccgtctccctaaacgggaccagactagcagctggatctccgtcgagatcatattcatctggggaaccatccaggaccacgcgaattcgcgtatcaccggtcgcagaaaacggcagaaatagtggtgctagtaaccgtgtgccattttctgccaccactacaacgactagaggaagagacgcgcactacaatgcagaaatacggacccatctttacatactatgggctgtgggtttattgctgggacttgtccttatactttacctgtgcgttccacgatgccggcgtaagaaaccctacatagtgtaa

SEQ ID NO: 55 - DNA, 687 bp, Human herpesvirus 8
  AF068829.11-687 Kaposi′s sarcoma-associated herpesvirus glycoprotein K8.1A mRNA, complete cds
atgagttccacacagattcgcacagaaatccctgtggcgctcctaatcctatgcctttgtctggtggcgtgccatgccaattgtcccacgtatcgttcgcatttgggattctggcaagagggttggagtggacaggtttatcaggactggctaggcaggatgaactgttcctacgagaatatgacggccctagaggccgtctccctaaacgggaccagactagcagctggatctccgtcgagtgagtatccaaatgtctccgtatctgtcgaagatacgtctgcctctgggtctggagaagatgcaatagatgaatcggggtcgggggaggaagagcgtcccgtgacctctcacgtgacttttatgacacaaagcgtccaggccaccacagaactgaccgatgccttaatatcagccttttcaggatcatattcatctggggaaccatccaggaccacgcgaattcgcgtatcaccggtcgcagaaaacggcagaaatagtggtgctagtaaccgtgtgccattttctgccaccactacaacgactagaggaagagacgcgcactacaatgcagaaatacggacccatctttacatactatgggctgtgggtttattgctgggacttgtccttatactttacctgtgcgttccacgatgccggcgtaagaaaccctacatagtgtaa

SEQ ID NO: 56 - DNA, 504 bp, Human herpesvirus 8; identical to SEQ ID NO: 54
  AF068830.11-504 Kaposi′s sarcoma-associated herpesvirus glycoprotein K8.1B mRNA, complete cds BCBL1 strain

SEQ ID NO: 57 - DNA, 2538 bp, Human herpesvirus 8 strain GK18
  NC_009333.18665-11202 Human herpesvirus 8 strain GK18 genome glycoprotein gB gene ORF8
atgactcccaggtctagattggccaccctggggactgtcatcctgttggtctgcttttgcgcaggcgcggcgcactcgaggggtgacacctttcagacgtccagttcccccacacccccaggatcttcctctaaggcccccaccaaacctggtgaggaagcatctggtcctaagagtgtggacttttaccagttcagagtgtgtagtgcatcgatcaccggggagctttttcggttcaacctggagcagacgtgcccagacaccaaagacaagtaccaccaagaaggaattttactggtgtacaaaaaaaacatagtgcctcatatctttaaggtgcggcgctataggaaaattgccacctctgtcacggtctacaggggcttgacagagtccgccatcaccaacaagtatgaactcccgagacccgtgccactctatgagataagccacatggacagcacctatcagtgctttagttccatgaaggtaaatgtcaacggggtagaaaacacatttactgacagagacgatgttaacaccacagtattcctccaaccagtagaggggcttacggataacattcaaaggtactttagccagccggtcatctacgcggaacccggctggtttcccggcatatacagagttaggaccaccgtcaattgcgagatagtggacatgatagccaggtctgctgaaccatacaattactttgtcacgtcactgggtgacacggtggaagtctcccctttttgctataacgaatcctcatgcagcacaacccccagcaacaaaaatggccttagcgtccaagtagttctcaaccacactgtggtcacgtactctgacagaggaaccagtcccactccccaaaacaggatctttgtggaaacgggagcgtacacgctttcgtgggcctccgagagcaagaccacggccgtgtgtccgctggcactgtggaaaaccttcccgcgctccatccagactacccacgaggacagcttccactttgtggccaacgagatcacggccaccttcacggctcctctaacgccagtggccaactttaccgacacgtactcttgtctgacctcggatatcaacaccacgctaaacgccagcaaggccaaactggcgagcactcacgtccctaacgggacggtccagtacttccacacaacaggcggactctatttggtctggcagcccatgtccgcgattaacctgactcacgctcagggcgacagcgggaaccccacgtcatcgccgcccccctccgcatcccccatgaccacctctgccagccgcagaaagagacggtcagccagtaccgctgctgccggcggcggggggtccacggacaacctgtcttacacgcagctgcagtttgcctacgacaaactgcgggatggcattaatcaggtgttagaagaactctccagggcatggtgtcgcgagcaggtcagggacaacctaatgtggtacgagctcagtaaaatcaaccccaccagcgttatgacagccatctacggtcgacctgtatccgccaagttcgtaggagacgccatttccgtgaccgagtgcattaacgtggaccagagctccgtaaacatccacaagagcctcagaaccaatagtaaggacgtgtgttacgcgcgccccctggtgacgtttaagtttttgaacagttccaacctattcaccggccagctgggcgcgcgcaatgagataatactgaccaacaaccaggtggaaacctgcaaagacacctgcgaacactacttcatcacccgcaacgagactctggtgtataaggactacgcgtacctgcgtactataaacaccactgacatatccaccctgaacacttttatcgccctgaatctatcctttattcaaaacatagacttcaaggccatcgagctgtacagcagtgcagagaaacgactcgcgagtagcgtgtttgacctggagacgatgttcagggagtacaactactacacacatcgtctcgcgggtttgcgcgaggatctggacaacaccatagatatgaacaaggagcgcttcgtaagggacttgtcggagatagtggcggacctgggtggcatcggaaaaacggtggtgaacgtggccagcagcgtggtcactctatgtggctcattggttaccggattcataaattttattaaacaccccctaggtggcatgctgatgatcattatcgttatagcaatcatcctgatcatttttatgctcagtcgccgcaccaataccatagcccaggcgccggtgaagatgatctaccccgacgtagatcgcagggcacctcctagcggcggagccccaacacgggaggaaatcaaaaacatcctgctgggaatgcaccagctacaacaagaggagaggcagaaggcggatgatctgaaaaaaagtacaccctcggtgtttcagcgtaccgcaaacggccttcgtcagcgtctgagaggatataaacctctgactcaatcgctagacatcagtccggaaacgggggagtga

SEQ ID NO: 58 - DNA, 2193 bp, Human herpesvirus 8 strain GK18
  NC_009333.137212-39404 Human herpesvirus 8 strain GK18 genome glycoprotein gH ORF22 gene
atgcagggtctagccttcttggcggcccttgcatgctggcgatgcatatcgttgacatgtggagccactggcgcgttgccgacaacggcgacgacaataacccgctccgccacgcagctcatcaatgggagaaccaacctctccatagaactggaattcaacggcactagtttttttctaaattggcaaaatctgttgaatgtgatcacggagccggccctgacagagttgtggacctccgccgaagtcgccgaggacctcagggtaactctgaaaaagaggcaaagtctttttttccccaacaagacagttgtgatctctggagacggccatcgctatacgtgcgaggtgccgacgtcgtcgcaaacttataacatcaccaagggctttaactatagcgctctgcccgggcaccttggcggatttgggatcaacgcgcgtctggtactgggtgatatcttcgcatcaaaatggtcgctattcgcgagggacaccccagagtatcgggtgttttacccaatgaatgtcatggccgtcaagttttccatatccattggcaacaacgagtccggcgtagcgctctatggagtggtgtcggaagatttcgtggtcgtcacgctccacaacaggtccaaagaggctaacgagacggcgtcccatcttctgttcggtctcccggattcactgccatctctgaagggccatgccacctatgatgaactcacgttcgcccgaaacgcaaaatatgcgctagtggcgatcctgcctaaagattcttaccagacactccttacagagaattacactcgcatatttctgaacatgacggagtcgacgcccctcgagttcacgcggacgatccagaccaggatcgtatcaatcgaggccaggcgcgcctgcgcagctcaagaggcggcgccggacatattcttggtgttgtttcagatgttggtggcacactttcttgttgcgcggggcattgccgagcaccgatttgtggaggtggactgcgtgtgtcggcagtatgcggaactgtattttctccgccgcatctcgcgtctgtgcatgcccacgttcaccactgtcgggtataaccacaccacccttggcgctgtggccgccacacaaatagctcgcgtgtccgccacgaagttggccagtttgccccgctcttcccaggaaacagtgctggccatggtccagcttggcgcccgtgatggcgccgtcccttcctccattctggagggcattgctatggtcgtcgaacatatgtataccgcctacacttatgtgtacacactcggcgatactgaaagaaaattaatgttggacatacacacggtcctcaccgacagctgcccgcccaaagactccggagtatcagaaaagctactgagaacatatttgatgttcacatcaatgtgtaccaacatagagctgggcgaaatgatcgcccgcttttccaaaccggacagccttaacatctatagggcattctccccctgctttctaggactaaggtacgatttgcatccagccaagttgcgcgccgaggcgccgcagtcgtccgctctgacgcggactgccgttgccagaggaacatcgggattcgcagaattgctccacgcgctgcacctcgatagcttaaatttaattccggcgattaactgttcaaagattacagccgacaagataatagctacggtacccttgcctcacgtcacgtatatcatcagttccgaagcactctcgaacgctgttgtctacgaggtgtcggagatcttcctcaagagtgccatgtttatatctgctatcaaacccgattgctccggctttaacttttctcagattgataggcacattcccatagtctacaacatcagcacaccaagaagaggttgccccctttgtgactctgtaatcatgagctacgatgagagcgatggcctgcagtctctcatgtatgtcactaatgaaagggtgcagaccaacctctttttagataagtcacctttctttgataataacaacctacacattcattatttgtggctgagggacaacgggaccgtagtggagataaggggcatgtatagaagacgcgcagccagtgctttgtttctaattctctcttttattgggttctcgggggttatctactttctttacagactgttttccatcctttattag

SEQ ID NO: 59 - DNA, 504 bp, Human herpesvirus 8 strain GK18
  NC_009333.1c70014-69511 Human herpesvirus 8 strain GK18genome, glycoprotein gL gene ORF47
atggggatctttgcgctatttgccgtcctgtggaccaccctattggtcacatctcacgcatacgtcgccttaccatgttgcgcaattcaggcatcggcagcctctaccctgccgttgttctttgcggtccactctatccacttcgccgatccgaatcactgcaacggggtctgtatagccaagctgcgaagcaaaacaggcgacattaccgtggaaacatgcgtgaatgggtttaatctgaggtcatttttagtcgcggtcgttcgaagattggggtcctgggcgtcgcaggaaaacctgaggttgttgtggtatttacaacgaagtttgacggcctatactgtaggttttaacgcgaccactgcagatagctctattcacaacgtaaacataattataataagcgtgggaaaggccatgaaccggacaggttctgttagcggaagtcagactcgggctaaaagcagcagccggagagcgcacgcaggtcaaaagggaaaataa

SEQ ID NO: 60 - DNA, 1185 bp, Human herpesvirus 1 strain 17
  NC_001806.2138423-139607 Human herpesvirus 1 strain 17,glycoprotein gD-US6
atggggggggctgccgccaggttgggggccgtgattttgtttgtcgtcatagtgggcctccatggggtccgcggcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactag

SEQ ID NO: 61 - DNA, 2715 bp, Human herpesvirus 1 strain 17
  NC_001806.2c55795-53081 Human herpesvirus 1 strain 17,glycoprotein gB-UL27
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 62 - DNA, 2450 bp, Human herpesvirus 1 strain 17
  NC_001806.2c46383-43867 Human herpesvirus 1 strain 17,glycoprotein gH-UL22
atggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgccgccggacgaactaaacctgactacggcatctctgccccttcttcgctggtacgaggagcgcttttgttttgtattggtcaccacggccgagtttccgcgggaccccggccagctgctttacatcccgaagacctacctgctcggccggcccccgaacgcgagcctgcccgcccccaccacggtcgagccgaccgcccagcctcccccctcggtcgccccccttaagggtctcttgcacaatccagccgcctccgtgttgctgcgttcccgggcctgggtaacgttttcggccgtccctgaccccgaggccctgacgttcccgcggggagacaacgtggcgacggcgagccacccgagcgggccgcgtgatacaccgcccccccgaccgccggttggggcccggcggcacccgacgacggagctggacatcacgcacctgcacaacgcgtccacgacctggttggccacccggggcctgttgagatccccaggtaggtacgtgtatttctccccgtcggcctcgacgtggcccgtgggcatctggacgacgggggagctggtgctcgggtgcgatgccgcgctggtgcgcgcgcgctacgggcgggaattcatggggctcgtgatatccatgcacgacagccctccggtggaagtgatggtggtccccgcgggccagacgctagatcgggtcggggaccccgcggacgaaaaccccccgggggctcttcccgggcccccgggcggcccccggtatcgggtctttgtcctagggtccctgacgcgggccgacaacggctccgcgctggacgccctccgccgcgtgggcggctacccggaggagggcacgaactacgcccagttcctgtcgcgggcatacgcggagtttttctcgggggacgcgggcgccgagcagggcccgcgcccccctctcttctggcgcctaacggggctgctcgcgacgtcgggttttgctttcgtgaacgccgcccacgcaaacggcgcggtctgcctctccgacctgctaggctttttggcccactcgcgcgcgcttgccgggttggccgcccgcggggccgcgggctgtgccgcggattctgtgttttttaatgtgtcagtcttggatcccacggcccgcctgcagctagaggctcggctccagcacctggtggccgagattctggagcgcgaacagagcttggcattacacgcgctgggctatcagctggccttcgtgctggatagcccctcggcgtacgacgcagtggcgcccagcgcagcccatctcatcgacgccctgtatgccgagtttctagggggccgcgtgctgaccaccccggtcgtccaccgggcgctattttacgcctcggctgtcctccggcagccgttcttggctggcgtcccctcggcggtgcagcgggaacgcgcccgccggagccttctgatagcctcggccctgtgtacgtccgacgtcgccgcagcgaccaacgccgacctccggaccgcgctggcccgggccgaccaccagaaaaccctcttttggcttccggaccacttttcgccatgcgcggcctccctgcgctttgatctagacgagagcgtgtttatcctggacgcgctggctcaagccacccgatccgagaccccggtcgaagtcctggcccagcagacccacggcctcgcctcgaccctgacgcgttgggcacactacaacgccctgatccgcgccttcgtccctgaggcctcacatcggtgcggggggcagtctgccaacgtcgagccacggatcctggtacccatcacccacaacgccagctacgtcgtcacccactcccctctgccccgggggatcggctacaagctcaccggcgtcgacgtccgacgcccactgttcctaacctacctcaccgcgacatgcgaaggctccacccgggatatcgagtccaagcggctggtgcgcacccaaaaccagcgcgacctggggctcgtgggggccgtgtttatgcgctacaccccggccggggaggtcatgtctgtgttgctggtggatacggacaacacacagcagcaaatcgccgccgggccgacggagggcgccccaagcgtgttttcgagcgacgtgccgtccacggccttgttgctatttccaaacggaaccgtcattcatttgctagcctttgacacgcagcccgtggccgcaattgcgcccgggtttctggccgcctctgcgctgggcgtggttatgattac

SEQ ID NO: 63 - DNA, 1185 bp, Artificial Sequence
  Rid1 based SNP in gD NC_001806.2138423-139607 Human herpesvirus 1 strain 17, glycoprotein gD-US6 Gln52Pro (mature Gln27) A155C
atggggggggctgccgccaggttgggggccgtgattttgtttgtcgtcatagtgggcctccatggggtccgcggcaaatatgccttggtggatgcctctctcaagatggccgaccccaatcgctttcgcggcaaagaccttccggtcctggacccgctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactag

SEQ ID NO: 64 - protein, 394 aa, Artificial Sequence
  Rid1 based SNP encoded amino acid sequence of SEQUENCE ID 63 with Gln52Pro
MGGAAARLGAVILFVVIVGLHGVRGKYALVDASLKMADPNRFRGKDLPVLDPLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 65 - DNA, 1182 bp, Artificial Sequence
  Rid 1 based SNP in NC_001798.2141016-142197 in Human herpesvirus2 strain HG52,glycoprotein D-US6 A155C
atggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggacccgctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgctaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggtgctggtcatcggcggtattgcgttttgggtacgccgccgcgctcagatggcccccaagcgcctacgtctcccccacatccgggatgacgacgcgcccccctcgcaccagccattgttttactag

SEQ ID NO: 66 - protein, 393 aa, Artificial Sequence
  Rid 1 based SNP encoded amino acid sequence of SEQUENCE ID 65 Gln52Pro
MGRLTSGVGTAALLVVAVGLRVVCAKYALADPSLKMADPNRFRGKNLPVLDPLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 67 - DNA, 2715 bp, Artificial Sequence
  Beta23 fold double amino acid SNPs in >NC_001806.2c55795-53081 Human herpesvirus 1 strain 17, glycoprotein gB-UL27 Q584A N585A 1750 CA-GC 1753 AA-GC
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtcgcagcctcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 68 - DNA, 2715 bp, Artificial Sequence
  Beta23 fold single amino acid SNPs encoded in >NC_001806.2c55795-53081 Human herpesvirus 1 strain 17,glycoprotein gB-UL27 Q584A N585A 1750 CA-GC 1753 AA-GC
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaagcctcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 69 - DNA, 2715 bp, Artificial Sequence
  Beta30 fold double amino acid SNPs on >NC_001806.2c55795-53081 Human herpesvirus 1 strain 17, glycoprotein gB-UL27 Y555A Q558A 1963TA-GC 1972-TA-GC
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcggcctcccacgcgctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 70 - DNA, 2715 bp, Artificial Sequence
  Beta30 fold single amino acid SNPs on >NC_001806.2c55795-53081 Human herpesvirus 1 strain 17, glycoprotein gB-UL27 Q558A 1972-TA-GC
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccacgcgctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 71 - DNA, 2715 bp, Artificial Sequence
  Beta23 fold double amino acid SNPS in NC_001798.2c56152-53438 Human herpesvirus2 strain HG52, glycoprotein B-UL27 1682 CA-GC 1685 AA-GC 581QN582 ?? AA
atgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtggcggcctcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 72 - DNA, 2715 bp, Artificial Sequence
  Beta23 fold single amino acid SNPs in NC_001798.2c56152-53438 Human herpesvirus2 strain HG52, glycoprotein B-UL27 ?? 1685 AA-GC N582A
atgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcaggcctcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 73 - DNA, 2715 bp, Artificial Sequence
  Beta30 fold double amino acid SNPs in NC_001798.2c56152-53438 Human herpesvirus2 strain HG52, glycoprotein B-UL27 Y652A Q655A 1954TA-GC 1963CA-GC
atgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcggcctctcacgcgctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 74 - DNA, 2715 bp, Artificial Sequence
  Beta30fold single amino acid SNPs in NC_001798.2c56152-53438 Human herpesvirus2 strain HG52, glycoprotein B-UL27 Q655A 1963CA-GC
atgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcacgcgctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 75 - DNA, 2796 bp, Artificial Sequence
  Beta23 fold double amino acid SNPS in NC_001348.156819-59614 Human herpesvirus 3 genome, glycoprotein gB Q596A N597A 1786CA-GC 1788AA-GC
atgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttgcagcctctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgaaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 76 - DNA, 2796 bp, Artificial Sequence
  Beta23 fold single amino acid SNPs in NC_001348.156819-59614 Human herpesvirus 3 genome, glycoprotein gB N597A 1789AA-GC
atgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttcaagcctctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgaaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 77 - DNA, 2796 bp, Artificial Sequence
  Beta30 fold double amino acid SNPS in NC_001348.156819-59614 Human herpesvirus 3 genome, glycoprotein gB Y667A E670A 1999TA-GC 2009A-C
atgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttcaaaactctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgtgccgtccgtgcaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 78 - DNA, 2796 bp, Artificial Sequence
  Beta30 fold single amino acid SNPs in NC_001348.156819-59614 Human herpesvirus 3 genome, glycoprotein gB E670A 2009A-C
atgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttcaaaactctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgcaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 79 - DNA, 2574 bp, Artificial Sequence
  Beta30 fold double amino acid SNPs in NC_009334.1c160348-157775 Human herpesvirus 4 genome, glycoprotein B BALF4 strain AG76 1900tt-gc 2013a-c F611A E615A
atgactcggcgtagggtgctaagcgtggtcgtgctgctagccgccctggcgtgccgcctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagaactaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacaccctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgccccccgccccacccgcggcccgcgggagcacctccgccgccgttctgaggcgccggaggcgggatgcggggaatgccaccacaccggtgccccccgcggcccccgggaagtccctgggcaccctcaacaatcccgccaccgtccagatccaatttgcctacgattccctgcgccgccagatcaaccgcatgctgggagacctcgcgcgggcctggtgcctggagcagaagaggcagaacatggtgctgagagaactaaccaagattaatcccaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccacgctaaaaccatcgcgctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagttttaa

SEQ ID NO: 80 - DNA, 2574 bp, Artificial Sequence
  Beta30fold single amino acid SNPs in NC_009334.1c160348-157775 Human herpesvirus 4 genome, glycoprotein B BALF4 strain AG76 2013a-c E615A
atgactcggcgtagggtgctaagcgtggtcgtgctgctagccgccctggcgtgccgcctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagaactaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacaccctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgccccccgccccacccgcggcccgcgggagcacctccgccgccgttctgaggcgccggaggcgggatgcggggaatgccaccacaccggtgccccccgcggcccccgggaagtccctgggcaccctcaacaatcccgccaccgtccagatccaatttgcctacgattccctgcgccgccagatcaaccgcatgctgggagacctcgcgcgggcctggtgcctggagcagaagaggcagaacatggtgctgagagaactaaccaagattaatcccaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccactttaaaaccatcgcgctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagttttaa

SEQ ID NO: 81 - DNA, 2724 bp, Artificial Sequence
  Beta30 fold double amino acid SNPs in NC_006273.2c84789-82066 Human herpesvirus 5 strain Merlin genome UL55 gB 1896tt-gc 1913a-c F633A D638A
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcgccaaacgcatgattgccctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctga

SEQ ID NO: 82 - DNA, 2724 bp, Artificial Sequence
  Beta30 fold single amino acid SNPs in NC_006273.2c84789-82066 Human herpesvirus 5 strain Merlin genome UL55 gB 1913a-c D638A
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgccctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctga

SEQ ID NO: 83 - DNA, 2423 bp, Artificial Sequence
  Beta30 fold double amino acid SNPs in NC_001664.4c62129-59637 Human betaherpesvirus 6A, variant A genome, strain U1102 gB U39 gene 1708ca-gc 1730a-c H671A E678A
cggatcattatatcagagcgggctataatcacaaatatccttttcggatttgttcgattgccaaaggcacggatttaatgcggttcgacagagatatttcgtgctcgccgtataagtctaatgcaaagatgtcggagggttttttcatcatttacaaaacaaatatcgagacctacacttttccagtgagaacgtataaaaaagagttgacgttccaaagtagttaccgtgatgtgggtgtggtttattttctggatcggacggtgatgggtttggccatgccggtgtacgaagcaaatttagttaattctcatgcgcagtgttattccgccgtagcgatgaaacgacccgatggtacggtgtttagtgcctttcatgaggataataataaaaacaatactctaaatttatttcctctgaatttcaaatctataactaataaaagatttatcactacgaaagaaccctactttgcaaggggtcctttgtggctatattctacatcaacgtctctcaattgtattgtgacggaggctacggctaaggcgaaatacccgtttagttactttgctttgacgactggcgaaatcgtggaagggtctccgttcttcaacggttcaaacggtaaacattttgcagagccgttagaaaaattgacaatcttggaaaactatactatgatagaagatctaatgaatggtatgaatggggccactacgttagtgaggaaaatcgcttttctcgagaaagcggatactttgttttcttgggaaatcaaggaagagaatgaatcggtgtgtatgctaaagcactggaccacggtgactcacgggcttcgagcggagacgaatgagacctatcactttatctctaaggagttgacagccgctttcgtcgcccccaaggagtccttaaatcttaccgatccgaaacaaacgtgtattaagaatgaatttgaaaaaataattaatgaagtctatatgtcagattataatgatacatatagcatgaatggtagttatcaaatttttaagactacgggagatttgattttgatttggcaacctcttgtgcaaaaatctcttatgtttcttgagcagggttcggaaaaaatacgtaggaggcgagatgtgggggatgttaagtctagacatgatattctttatgtgcaattacagtatctttatgatactttgaaagattatatcaatgatgcattggggaatttggcagaatcttggtgtctcgatcaaaagcgaacgataacgatgttgcacgaacttagtaagattagtccgtcgagtatcgtgtctgaggtttacggtcgtccgatatctgcacagttgcatggtgacgtgttagctatctcgaaatgcatagaggttaatcagtcatccgttcagcttcataagagtatgcgggtcgtcgatgcaaagggagtaaggagtgaaacgatgtgttataatcggcccttggtgacgtttagttttgtgaactcgacgcctgaggttgtccctggccagctagggttagataatgaaattctgttgggtgatcataggacagaggaatgtgaaatacctagtacaaagatctttttatctggaaatcatgcgcacgtgtataccgattatacggctacgaattcgacgcccatagcagacattgaggtattggatgcttttattagactaaaaatcgatcctcttgaaaatgccgattttaaagtactcgatttatattcgccggacgaattgagtagagcaaacgttttcgatctagagaatattcttcgtgaatataactcatataagagcgcactatatactatagaagctaaaattgctacgaatacaccttcgtatgttaatgggattaattcttttttacaagggcttggggctataggcactggattgggctcggttataagtgttacggcaggagcgcttggggatattgtgggtggagtggtgtcttttctaaaaaatccattcgggggtggtctcatgttgattttagcgatagtagttgttgtgataataattgtggttttcgttagacaaagacatgtgcttagtaagcctattgacatgatgtttccttatgccaccaatccggtgactactgtgtccagtgttacggggaccactgtcgtcaagacacctagtgttaaggatgttgacgggggcacatctgttgcggtttcggaaaaggaggagggtatggctgacgtcagtgggcaagtaagtgacgatgaatattcacaagaagatgctttaaaaatgctcaaggccataaagtccttagacgagtcctacagaagaaaaccttcgtcttctgagtctcatgcctcaaaacctagtttgatagacaggatcaggtatagaggttataagagtgtaaatgtagaagaagcgtga

SEQ ID NO: 84 - DNA, 2493 bp, Artificial Sequence
  Beta30 fold single amino acid SNPs in NC_001664.4c62129-59637 Human betaherpesvirus 6A, variant A DNA, virion genome, strain U1102 glycoprotein gB U39 gene 1730a-c E678A
atgagcaagatggcagtattattcctggctgtctttttgatgaatagtgttttaatgatatattgtgatccggatcattatatcagagcgggctataatcacaaatatccttttcggatttgttcgattgccaaaggcacggatttaatgcggttcgacagagatatttcgtgctcgccgtataagtctaatgcaaagatgtcggagggttttttcatcatttacaaaacaaatatcgagacctacacttttccagtgagaacgtataaaaaagagttgacgttccaaagtagttaccgtgatgtgggtgtggtttattttctggatcggacggtgatgggtttggccatgccggtgtacgaagcaaatttagttaattctcatgcgcagtgttattccgccgtagcgatgaaacgacccgatggtacggtgtttagtgcctttcatgaggataataataaaaacaatactctaaatttatttcctctgaatttcaaatctataactaataaaagatttatcactacgaaagaaccctactttgcaaggggtcctttgtggctatattctacatcaacgtctctcaattgtattgtgacggaggctacggctaaggcgaaatacccgtttagttactttgctttgacgactggcgaaatcgtggaagggtctccgttcttcaacggttcaaacggtaaacattttgcagagccgttagaaaaattgacaatcttggaaaactatactatgatagaagatctaatgaatggtatgaatggggccactacgttagtgaggaaaatcgcttttctcgagaaagcggatactttgttttcttgggaaatcaaggaagagaatgaatcggtgtgtatgctaaagcactggaccacggtgactcacgggcttcgagcggagacgaatgagacctatcactttatctctaaggagttgacagccgctttcgtcgcccccaaggagtccttaaatcttaccgatccgaaacaaacgtgtattaagaatgaatttgaaaaaataattaatgaagtctatatgtcagattataatgatacatatagcatgaatggtagttatcaaatttttaagactacgggagatttgattttgatttggcaacctcttgtgcaaaaatctcttatgtttcttgagcagggttcggaaaaaatacgtaggaggcgagatgtgggggatgttaagtctagacatgatattctttatgtgcaattacagtatctttatgatactttgaaagattatatcaatgatgcattggggaatttggcagaatcttggtgtctcgatcaaaagcgaacgataacgatgttgcacgaacttagtaagattagtccgtcgagtatcgtgtctgaggtttacggtcgtccgatatctgcacagttgcatggtgacgtgttagctatctcgaaatgcatagaggttaatcagtcatccgttcagcttcataagagtatgcgggtcgtcgatgcaaagggagtaaggagtgaaacgatgtgttataatcggcccttggtgacgtttagttttgtgaactcgacgcctgaggttgtccctggccagctagggttagataatgaaattctgttgggtgatcataggacagaggaatgtgaaatacctagtacaaagatctttttatctggaaatcatgcgcacgtgtataccgattatacgcatacgaattcgacgcccatagcagacattgaggtattggatgcttttattagactaaaaatcgatcctcttgaaaatgccgattttaaagtactcgatttatattcgccggacgaattgagtagagcaaacgttttcgatctagagaatattcttcgtgaatataactcatataagagcgcactatatactatagaagctaaaattgctacgaatacaccttcgtatgttaatgggattaattcttttttacaagggcttggggctataggcactggattgggctcggttataagtgttacggcaggagcgcttggggatattgtgggtggagtggtgtcttttctaaaaaatccattcgggggtggtctcatgttgattttagcgatagtagttgttgtgataataattgtggttttcgttagacaaagacatgtgcttagtaagcctattgacatgatgtttccttatgccaccaatccggtgactactgtgtccagtgttacggggaccactgtcgtcaagacacctagtgttaaggatgttgacgggggcacatctgttgcggtttcggaaaaggaggagggtatggctgacgtcagtgggcaagtaagtgacgatgaatattcacaagaagatgctttaaaaatgctcaaggccataaagtccttagacgagtcctacagaagaaaaccttcgtcttctgagtctcatgcctcaaaacctagtttgatagacaggatcaggtatagaggttataagagtgtaaatgtagaagaagcgtga

SEQ ID NO: 85 - DNA, 2493 bp, Artificial Sequence
  Beta30 fold double amino acid SNPs in NC_000898.1c63200-60708 Human herpesvirus 6B, strain Z29 genome gB U39 gene 1708ca-gc 1730a-c H670A E677A
atgagcaagatgagagtattattcctggctgtctttttgatgaatagtgttttaatgatatattgcgattcggatgattatatcagagcgggctataatcacaaatatccttttcggatttgttcgattgccaaaggcactgatttgatgcggttcgacagagatatttcgtgttcgccatataagtctaatgcaaagatgtcggagggttttttcatcatttacaaaacaaatatcgagacctacacttttccagtgagaacatataaaaacgagctgacgttcccaaccagttaccgtgatgtgggtgtggtttattttctggatcggacggtgatgggtttggccatgccggtgtacgaagcaaatttagttaattctcgtgcgcagtgttattcagccgtagcgataaaacgacccgatggtacggtgtttagtgcctatcatgaggataataataaaaacgaaactctagaattatttcctctgaatttcaagtctgttactaataaaagatttatcactacgaaagaaccctactttgcaaggggtcctttgtggctctattctacatcgacgtctctcaattgtattgtgacggaggctacggctaaggcgaaatatccgtttagttactttgctttgacgactggtgaaatcgtggaagggtctccgttcttcgacggttcaaacggtaaacattttgcagagccgttagaaaaattgacaatcttggaaaactatactatgatagaagatctaatgaatggtatgaatggggctactacgttagtaaggaagatcgcttttctggagaaaggggatactttgttttcttgggaaatcaaggaagagaatgaatcggtgtgtatgctaaagcactggactacggtgactcacgggcttcgagcggagacggatgagacttatcactttatttctaaggagttgacagccgctttcgtcgcctccaaggagtctttaaatcttaccgatcccaaacaaacgtgtattaagaatgaatttgagaagataattacagatgtctatatgtcagattataatgatgcatacagcatgaacggtagttatcaaatttttaagactacgggagatctgattttgatttggcagcctcttgtgcaaaaatctcttatggttcttgagcagggttcagtaaacttacgtaggaggcgagatttggtggatgtcaagtctagacatgatattctttatgtgcaattacagtacctctatgatactttgaaagattatatcaacgatgccttggggaatttggcagaatcttggtgcctcgatcaaaaacgaacgataacgatgttgcacgaacttagtaagatcagtccatcgagtatcgtgtctgaggtttacggtcgtccgatatctgcacagttgcatggtgatgtgttagctatctcgaaatgcatagaagttaatcaatcatccgttcagctttataagagtatgcgggtcgtcgatgcgaagggagtaaggagtgaaacgatgtgttataatcggcccttggtgacgtttagctttgtgaactccacgcctgaggttgtccttggtcagctagggttagataatgagattctgttgggtgatcataggacagaggaatgtgagatacctagtacaaagatatttctatctggaaatcatgcacacgtgtataccgattatacggctacgaattcgacgcccatagcagacattgaggtattggatgcttttattagactaaagatcgaccctctcgaaaatgctgattttaaactacttgatttatattcgccggacgaattgagtagagcaaacgttttcgatttagagaatattcttcgtgaatataactcatataagagcgcactatatactatagaagctaaaattgctactaatacgccgtcgtatgtcaatgggattaattcttttttacaagggcttggggctataggcactggattgggctcggttataagtgttacggcaggagcacttggggatattgtgggtggagtggtgtcttttttaaaaaatccattcgggggtggtctcatgttgattttagcgatagtagttgtcgttataataattgtggttttcgttagacaaaaacatgtgcttagtaagcctattgacatgatgtttccttatgccaccaatccggtgactactgtgtccagtgttacggggaccactgtcgtcaagacgcctagtgttaaagatgctgacgggggcacatctgttgcggtttcggaaaaagaggagggtatggctgacgtcagtggacaaataagtggtgatgaatattcacaagaagatgctttaaaaatgctcaaggccataaagtctttagacgagtcctacagaagaaaaccttcgtcttctgagtctcatgcctcaaaacctagtttgatagacaggatcaggtatagaggttataagagtgtaaatgtagaagaagcgtga

SEQ ID NO: 86 - DNA, 2493 bp, Artificial Sequence
  Beta30 fold single amino acid SNPs in gB NC_000898.1c63200-60708 Human herpesvirus 6B, strain Z29 genome gB U39 gene put CA back - 1730a-c E677A
atgagcaagatgagagtattattcctggctgtctttttgatgaatagtgttttaatgatatattgcgattcggatgattatatcagagcgggctataatcacaaatatccttttcggatttgttcgattgccaaaggcactgatttgatgcggttcgacagagatatttcgtgttcgccatataagtctaatgcaaagatgtcggagggttttttcatcatttacaaaacaaatatcgagacctacacttttccagtgagaacatataaaaacgagctgacgttcccaaccagttaccgtgatgtgggtgtggtttattttctggatcggacggtgatgggtttggccatgccggtgtacgaagcaaatttagttaattctcgtgcgcagtgttattcagccgtagcgataaaacgacccgatggtacggtgtttagtgcctatcatgaggataataataaaaacgaaactctagaattatttcctctgaatttcaagtctgttactaataaaagatttatcactacgaaagaaccctactttgcaaggggtcctttgtggctctattctacatcgacgtctctcaattgtattgtgacggaggctacggctaaggcgaaatatccgtttagttactttgctttgacgactggtgaaatcgtggaagggtctccgttcttcgacggttcaaacggtaaacattttgcagagccgttagaaaaattgacaatcttggaaaactatactatgatagaagatctaatgaatggtatgaatggggctactacgttagtaaggaagatcgcttttctggagaaaggggatactttgttttcttgggaaatcaaggaagagaatgaatcggtgtgtatgctaaagcactggactacggtgactcacgggcttcgagcggagacggatgagacttatcactttatttctaaggagttgacagccgctttcgtcgcctccaaggagtctttaaatcttaccgatcccaaacaaacgtgtattaagaatgaatttgagaagataattacagatgtctatatgtcagattataatgatgcatacagcatgaacggtagttatcaaatttttaagactacgggagatctgattttgatttggcagcctcttgtgcaaaaatctcttatggttcttgagcagggttcagtaaacttacgtaggaggcgagatttggtggatgtcaagtctagacatgatattctttatgtgcaattacagtacctctatgatactttgaaagattatatcaacgatgccttggggaatttggcagaatcttggtgcctcgatcaaaaacgaacgataacgatgttgcacgaacttagtaagatcagtccatcgagtatcgtgtctgaggtttacggtcgtccgatatctgcacagttgcatggtgatgtgttagctatctcgaaatgcatagaagttaatcaatcatccgttcagctttataagagtatgcgggtcgtcgatgcgaagggagtaaggagtgaaacgatgtgttataatcggcccttggtgacgtttagctttgtgaactccacgcctgaggttgtccttggtcagctagggttagataatgagattctgttgggtgatcataggacagaggaatgtgagatacctagtacaaagatatttctatctggaaatcatgcacacgtgtataccgattatacgcatacgaattcgacgcccatagcagacattgaggtattggatgcttttattagactaaagatcgaccctctcgaaaatgctgattttaaactacttgatttatattcgccggacgaattgagtagagcaaacgttttcgatttagagaatattcttcgtgaatataactcatataagagcgcactatatactatagaagctaaaattgctactaatacgccgtcgtatgtcaatgggattaattcttttttacaagggcttggggctataggcactggattgggctcggttataagtgttacggcaggagcacttggggatattgtgggtggagtggtgtcttttttaaaaaatccattcgggggtggtctcatgttgattttagcgatagtagttgtcgttataataattgtggttttcgttagacaaaaacatgtgcttagtaagcctattgacatgatgtttccttatgccaccaatccggtgactactgtgtccagtgttacggggaccactgtcgtcaagacgcctagtgttaaagatgctgacgggggcacatctgttgcggtttcggaaaaagaggagggtatggctgacgtcagtggacaaataagtggtgatgaatattcacaagaagatgctttaaaaatgctcaaggccataaagtctttagacgagtcctacagaagaaaaccttcgtcttctgagtctcatgcctcaaaacctagtttgatagacaggatcaggtatagaggttataagagtgtaaatgtagaagaagcgtga

SEQ ID NO: 87 - DNA, 2469 bp, Artificial Sequence
  Beta30 fold double amino acid SNPs in NC_001716.2c61093-58625 Human herpesvirus 7genome, glycoprotein gB U39 1693ta-gc 1718a-c Y665A E673A
atgaaaattctattcctgagtgtttttataacttttagcttacagctatctctacaaacagaagctgactttgtcatgactggacacaatcagcatttaccatttcgaatttgttcaattgccaccgggacagatttagtgcgttttgacagggaggtttcttgcgcgtcttacggctctaacattaaaactaccgaaggtattttgataatttacaaaacaaagattgaagcacacacgttttctgtcagaacttttaaaaaagaacttacatttcaaacaacatatcgcgatgttggcactgtgtatttcttagatcgaactgttacaactttgccaatgccaattgaagaagtgcacatggtaaacaccgaggcgcgttgtttgtcctctatctctgtaaaacgttcggaggaagaggagtatgttgcatatcataaagatgaatatgtgaataaaacgttggatttgattccgttaaattttaaatctgatactgtcagaagatatataactactaaagaaccatttttaagaaatggtcccctctggttctattcaacatctacatcgataaattgcatagttacagactgcattgcaaagactaaatatccttttgatttttttgctttatcaacaggggaaaccgtggaagggtcaccgttttataatggtattaattctaaaacatttaatgaaccaacggaaaaaattttgtttagaaataattatactatgctgaaaacgtttgatgatggatcaaaaggaaattttgtgacgttaactaaaatggcttttctggaaaagggaaatactattttttcatgggaagtgcagaatgaagagtcttccatttgtttgttaaagcattggatgacaatcccacacgctttacgggcagaaaatgctaacagttttcactttattgcgcaggaactaactgcttcttttgtcacaggaaaaagtaattatacgctttctgattcaaaatataattgtattaacagcaattatacttcaattttggatgagatttaccaaacgcaatacaacaattcgcatgacaaaaatggtagttatgaaatttttaaaactgagggagatttaattctgatttggcaaccgttaatacaacggaaattaacggttttggaaaatttttctaatgcttctagaaaaaggaggaagagggaattagagactaacaaagacatcgtatatgttcaactacaatacctatacgacactctgaaagattacattaacacagcactaggtaagcttgctgaagcctggtgtttagatcaaaaacgcactatcacagtgttgcacgagcttagtaaaataagtccttctggaatcatttcagcagtgtacggtaaacccatgtccgctaaattaattggtgatgtattagcagtctcaaagtgcattgaagtcaaccagacttctgttcagctacataaaagtatgagattgacaaaagattcaagttacgatgctctaagatgttattctcggccattgttaacatattcatttgcaaattcttcgaaggaaacttatttaggacaacttggtttagacaatgagatattactgggaaatcacagaacagaagaatgtgaacaatctaacacgaaaatttttttgtctggtaaattcgctcatatttttaaagactatacggccgttaattctagtttgataacagcaatagaagctttagatgcgtttgttgacttaaacatagatcctttagaaaatgcagattttacattattggaattgtatacaaaagatgaactgagcaaagcgaacgtttttgatttggaaaccattcttagagaatacaattcttacaaaagtgcgctacaccatatagaaacaaaaattgcaactgttactccaacatatataggagggattgacacatttttcaaaggtcttggcgctcttggtcttggtttgggggctgtgctgggtgtaacggctggtgctttgggagatgtggtgaatggagttttctcttttcttaaaaatccatttggtggagcactaactatcttactaactttgggagtgattggcttagttattttcttatttttaagacataaacgattagcacaaacaccgattgatattttatttccttatacatcaaaatcaacaaattcggtacttcaagcaacgcaatcagttcaagcgcaagtgaaagaacctttagattcatctccgccttatttaaaaactaacaaagacacagaaccgcaaggtgatgacataacacacactaatgaatattcacaagtggaagctttaaaaatgttgaaagctattaaattattagatgagtcatataaaaaagcggaaatcgctgaggcaaaaaaatcacagagaccaagcctacttgaaaggattcaatacaggggctatcagaaactttcaacagaagaactgtga

SEQ ID NO: 88 - DNA, 2469 bp, Artificial Sequence
  Beta30 fold single amino acid SNPs in NC_001716.2c61093-58625 Human herpesvirus 7genome, glycoprotein gB U39 ta-gc a-c E673A
atgaaaattctattcctgagtgtttttataacttttagcttacagctatctctacaaacagaagctgactttgtcatgactggacacaatcagcatttaccatttcgaatttgttcaattgccaccgggacagatttagtgcgttttgacagggaggtttcttgcgcgtcttacggctctaacattaaaactaccgaaggtattttgataatttacaaaacaaagattgaagcacacacgttttctgtcagaacttttaaaaaagaacttacatttcaaacaacatatcgcgatgttggcactgtgtatttcttagatcgaactgttacaactttgccaatgccaattgaagaagtgcacatggtaaacaccgaggcgcgttgtttgtcctctatctctgtaaaacgttcggaggaagaggagtatgttgcatatcataaagatgaatatgtgaataaaacgttggatttgattccgttaaattttaaatctgatactgtcagaagatatataactactaaagaaccatttttaagaaatggtcccctctggttctattcaacatctacatcgataaattgcatagttacagactgcattgcaaagactaaatatccttttgatttttttgctttatcaacaggggaaaccgtggaagggtcaccgttttataatggtattaattctaaaacatttaatgaaccaacggaaaaaattttgtttagaaataattatactatgctgaaaacgtttgatgatggatcaaaaggaaattttgtgacgttaactaaaatggcttttctggaaaagggaaatactattttttcatgggaagtgcagaatgaagagtcttccatttgtttgttaaagcattggatgacaatcccacacgctttacgggcagaaaatgctaacagttttcactttattgcgcaggaactaactgcttcttttgtcacaggaaaaagtaattatacgctttctgattcaaaatataattgtattaacagcaattatacttcaattttggatgagatttaccaaacgcaatacaacaattcgcatgacaaaaatggtagttatgaaatttttaaaactgagggagatttaattctgatttggcaaccgttaatacaacggaaattaacggttttggaaaatttttctaatgcttctagaaaaaggaggaagagggaattagagactaacaaagacatcgtatatgttcaactacaatacctatacgacactctgaaagattacattaacacagcactaggtaagcttgctgaagcctggtgtttagatcaaaaacgcactatcacagtgttgcacgagcttagtaaaataagtccttctggaatcatttcagcagtgtacggtaaacccatgtccgctaaattaattggtgatgtattagcagtctcaaagtgcattgaagtcaaccagacttctgttcagctacataaaagtatgagattgacaaaagattcaagttacgatgctctaagatgttattctcggccattgttaacatattcatttgcaaattcttcgaaggaaacttatttaggacaacttggtttagacaatgagatattactgggaaatcacagaacagaagaatgtgaacaatctaacacgaaaatttttttgtctggtaaattcgctcatatttttaaagactatacgtacgttaattctagtttgataacagcaatagaagctttagatgcgtttgttgacttaaacatagatcctttagaaaatgcagattttacattattggaattgtatacaaaagatgaactgagcaaagcgaacgtttttgatttggaaaccattcttagagaatacaattcttacaaaagtgcgctacaccatatagaaacaaaaattgcaactgttactccaacatatataggagggattgacacatttttcaaaggtcttggcgctcttggtcttggtttgggggctgtgctgggtgtaacggctggtgctttgggagatgtggtgaatggagttttctcttttcttaaaaatccatttggtggagcactaactatcttactaactttgggagtgattggcttagttattttcttatttttaagacataaacgattagcacaaacaccgattgatattttatttccttatacatcaaaatcaacaaattcggtacttcaagcaacgcaatcagttcaagcgcaagtgaaagaacctttagattcatctccgccttatttaaaaactaacaaagacacagaaccgcaaggtgatgacataacacacactaatgaatattcacaagtggaagctttaaaaatgttgaaagctattaaattattagatgagtcatataaaaaagcggaaatcgctgaggcaaaaaaatcacagagaccaagcctacttgaaaggattcaatacaggggctatcagaaactttcaacagaagaactgtga

SEQ ID NO: 89 - DNA, 2538 bp, Artificial Sequence
  Beta30 fold double amino acid SNPs in NC_009333.18665-11202 Human herpesvirus 8 strain GK18 genome glycoprotein gB gene ORF8 1825ta-gc 1840aa-gc Y608A N613A
atgactcccaggtctagattggccaccctggggactgtcatcctgttggtctgcttttgcgcaggcgcggcgcactcgaggggtgacacctttcagacgtccagttcccccacacccccaggatcttcctctaaggcccccaccaaacctggtgaggaagcatctggtcctaagagtgtggacttttaccagttcagagtgtgtagtgcatcgatcaccggggagctttttcggttcaacctggagcagacgtgcccagacaccaaagacaagtaccaccaagaaggaattttactggtgtacaaaaaaaacatagtgcctcatatctttaaggtgcggcgctataggaaaattgccacctctgtcacggtctacaggggcttgacagagtccgccatcaccaacaagtatgaactcccgagacccgtgccactctatgagataagccacatggacagcacctatcagtgctttagttccatgaaggtaaatgtcaacggggtagaaaacacatttactgacagagacgatgttaacaccacagtattcctccaaccagtagaggggcttacggataacattcaaaggtactttagccagccggtcatctacgcggaacccggctggtttcccggcatatacagagttaggaccaccgtcaattgcgagatagtggacatgatagccaggtctgctgaaccatacaattactttgtcacgtcactgggtgacacggtggaagtctcccctttttgctataacgaatcctcatgcagcacaacccccagcaacaaaaatggccttagcgtccaagtagttctcaaccacactgtggtcacgtactctgacagaggaaccagtcccactccccaaaacaggatctttgtggaaacgggagcgtacacgctttcgtgggcctccgagagcaagaccacggccgtgtgtccgctggcactgtggaaaaccttcccgcgctccatccagactacccacgaggacagcttccactttgtggccaacgagatcacggccaccttcacggctcctctaacgccagtggccaactttaccgacacgtactcttgtctgacctcggatatcaacaccacgctaaacgccagcaaggccaaactggcgagcactcacgtccctaacgggacggtccagtacttccacacaacaggcggactctatttggtctggcagcccatgtccgcgattaacctgactcacgctcagggcgacagcgggaaccccacgtcatcgccgcccccctccgcatcccccatgaccacctctgccagccgcagaaagagacggtcagccagtaccgctgctgccggcggcggggggtccacggacaacctgtcttacacgcagctgcagtttgcctacgacaaactgcgggatggcattaatcaggtgttagaagaactctccagggcatggtgtcgcgagcaggtcagggacaacctaatgtggtacgagctcagtaaaatcaaccccaccagcgttatgacagccatctacggtcgacctgtatccgccaagttcgtaggagacgccatttccgtgaccgagtgcattaacgtggaccagagctccgtaaacatccacaagagcctcagaaccaatagtaaggacgtgtgttacgcgcgccccctggtgacgtttaagtttttgaacagttccaacctattcaccggccagctgggcgcgcgcaatgagataatactgaccaacaaccaggtggaaacctgcaaagacacctgcgaacactacttcatcacccgcaacgagactctggtgtataaggactacgcggccctgcgtactatagccaccactgacatatccaccctgaacacttttatcgccctgaatctatcctttattcaaaacatagacttcaaggccatcgagctgtacagcagtgcagagaaacgactcgcgagtagcgtgtttgacctggagacgatgttcagggagtacaactactacacacatcgtctcgcgggtttgcgcgaggatctggacaacaccatagatatgaacaaggagcgcttcgtaagggacttgtcggagatagtggcggacctgggtggcatcggaaaaacggtggtgaacgtggccagcagcgtggtcactctatgtggctcattggttaccggattcataaattttattaaacaccccctaggtggcatgctgatgatcattatcgttatagcaatcatcctgatcatttttatgctcagtcgccgcaccaataccatagcccaggcgccggtgaagatgatctaccccgacgtagatcgcagggcacctcctagcggcggagccccaacacgggaggaaatcaaaaacatcctgctgggaatgcaccagctacaacaagaggagaggcagaaggcggatgatctgaaaaaaagtacaccctcggtgtttcagcgtaccgcaaacggccttcgtcagcgtctgagaggatataaacctctgactcaatcgctagacatcagtccggaaacgggggagtga

SEQ ID NO: 90 - DNA, 2538 bp, Artificial Sequence
  Beta30 fold single amino acid SNPs in NC_009333.18665-11202 Human herpesvirus 8 strain GK18 genome gB gene ORF8 1840aa-gc N613A
atgactcccaggtctagattggccaccctggggactgtcatcctgttggtctgcttttgcgcaggcgcggcgcactcgaggggtgacacctttcagacgtccagttcccccacacccccaggatcttcctctaaggcccccaccaaacctggtgaggaagcatctggtcctaagagtgtggacttttaccagttcagagtgtgtagtgcatcgatcaccggggagctttttcggttcaacctggagcagacgtgcccagacaccaaagacaagtaccaccaagaaggaattttactggtgtacaaaaaaaacatagtgcctcatatctttaaggtgcggcgctataggaaaattgccacctctgtcacggtctacaggggcttgacagagtccgccatcaccaacaagtatgaactcccgagacccgtgccactctatgagataagccacatggacagcacctatcagtgctttagttccatgaaggtaaatgtcaacggggtagaaaacacatttactgacagagacgatgttaacaccacagtattcctccaaccagtagaggggcttacggataacattcaaaggtactttagccagccggtcatctacgcggaacccggctggtttcccggcatatacagagttaggaccaccgtcaattgcgagatagtggacatgatagccaggtctgctgaaccatacaattactttgtcacgtcactgggtgacacggtggaagtctcccctttttgctataacgaatcctcatgcagcacaacccccagcaacaaaaatggccttagcgtccaagtagttctcaaccacactgtggtcacgtactctgacagaggaaccagtcccactccccaaaacaggatctttgtggaaacgggagcgtacacgctttcgtgggcctccgagagcaagaccacggccgtgtgtccgctggcactgtggaaaaccttcccgcgctccatccagactacccacgaggacagcttccactttgtggccaacgagatcacggccaccttcacggctcctctaacgccagtggccaactttaccgacacgtactcttgtctgacctcggatatcaacaccacgctaaacgccagcaaggccaaactggcgagcactcacgtccctaacgggacggtccagtacttccacacaacaggcggactctatttggtctggcagcccatgtccgcgattaacctgactcacgctcagggcgacagcgggaaccccacgtcatcgccgcccccctccgcatcccccatgaccacctctgccagccgcagaaagagacggtcagccagtaccgctgctgccggcggcggggggtccacggacaacctgtcttacacgcagctgcagtttgcctacgacaaactgcgggatggcattaatcaggtgttagaagaactctccagggcatggtgtcgcgagcaggtcagggacaacctaatgtggtacgagctcagtaaaatcaaccccaccagcgttatgacagccatctacggtcgacctgtatccgccaagttcgtaggagacgccatttccgtgaccgagtgcattaacgtggaccagagctccgtaaacatccacaagagcctcagaaccaatagtaaggacgtgtgttacgcgcgccccctggtgacgtttaagtttttgaacagttccaacctattcaccggccagctgggcgcgcgcaatgagataatactgaccaacaaccaggtggaaacctgcaaagacacctgcgaacactacttcatcacccgcaacgagactctggtgtataaggactacgcgtacctgcgtactatagccaccactgacatatccaccctgaacacttttatcgccctgaatctatcctttattcaaaacatagacttcaaggccatcgagctgtacagcagtgcagagaaacgactcgcgagtagcgtgtttgacctggagacgatgttcagggagtacaactactacacacatcgtctcgcgggtttgcgcgaggatctggacaacaccatagatatgaacaaggagcgcttcgtaagggacttgtcggagatagtggcggacctgggtggcatcggaaaaacggtggtgaacgtggccagcagcgtggtcactctatgtggctcattggttaccggattcataaattttattaaacaccccctaggtggcatgctgatgatcattatcgttatagcaatcatcctgatcatttttatgctcagtcgccgcaccaataccatagcccaggcgccggtgaagatgatctaccccgacgtagatcgcagggcacctcctagcggcggagccccaacacgggaggaaatcaaaaacatcctgctgggaatgcaccagctacaacaagaggagaggcagaaggcggatgatctgaaaaaaagtacaccctcggtgtttcagcgtaccgcaaacggccttcgtcagcgtctgagaggatataaacctctgactcaatcgctagacatcagtccggaaacgggggagtga

SEQ ID NO: 91 - DNA, 2715 bp, Artificial Sequence
  DomainI SNP A799G gB1 HSV1 strain 17
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacggggcgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 92 - DNA, 2796 bp, Artificial Sequence
  DomainI SNP A817G gB3 VZV strain Dumas
atgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcgcgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttcaaaactctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgaaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 93 - DNA, 2574 bp, Artificial Sequence
  DomainI SNP A604G gB4-1 EBV type 1 strain B95-8/Raji
atgactcggcgtagggtgctaagcgtggtcgtgctgctagccgccctggcgtgccgtctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagagctaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacacgctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgcccccagccccatccgcggcccgcgggagcacccccgccgccgttctgaggcgtcggaggcgggatgcggggaacgccaccacaccggtgccccccacggcccccgggaagtccctgggcaccctcaacaatcccgccaccgtccagatccaatttgcctacgactccctgcgccgccagatcaaccgcatgctgggagaccttgcgcgggcctggtgcctggagcagaagaggcagaacatggtgctgagagaactaaccaagattaatccaaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccactttaaaaccatcgagctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagttttaa

SEQ ID NO: 94 - DNA, 2574 bp, Artificial Sequence
  DomainI SNP A604G gB4-2 EBV type 2 strain AG876
atgactcggcgtagggtgctaagcgtggtcgtgctgctagccgccctggcgtgccgcctcggtgcgcagaccccagagcagcccgcaccccccgccaccacggtgcagcctaccgccacgcgtcagcaaaccagctttcctttccgagtctgcgagctctccagccacggcgacctgttccgcttctcctcggacatccagtgtccctcgtttggcacgcgggagaatcacacggagggcctgttgatggtgtttaaagacaacattattccctactcgtttaaggtccgctcctacaccaagatagtgaccaacattctcatctacaatggctggtacgcggactccgtgaccaaccggcacgaggagaagttctccgttgacagctacgaaactgaccagatggataccatctaccagtgctacaacgcggtcaagatgacaaaagatgggctgacgcgcgtgtatgtagaccgcgacggagttaacatcaccgtcaacctaaagcccaccgggggcctggccaacggggtgcgccgctacgccagccagacggagctctatgacgcccccgggtggttgatatggacttacagaacaagagctaccgtcaactgcctgataactgacatgatggccaagtccaacagccccttcgacttctttgtgaccaccaccgggcagactgtggaaatgtcccctttctatgacgggaaaaataaggaaaccttccatgagcgggcagactccttccacgtgagaactaactacaagatagtggactacgacaaccgagggacgaacccgcaaggcgaacgccgagccttcctggacaagggcacttacaccctatcttggaagctcgagaacaggacagcctactgcccgcttcaacactggcaaacctttgactcgaccatcgccacagaaacagggaagtcaatacattttgtgactgacgagggcacctctagcttcgtgaccaacacaaccgtgggcatagagctcccggacgccttcaagtgcatcgaagagcaggtgaacaagaccatgcatgagaagtacgaggccgtccaggatcgttacacgaagggccaggaagccattacatattttataacgagcggaggattgttattagcttggctacctctgaccccgcgctcgttggccaccgtcaagaacctgacggagcttaccactccgacttcctcaccccccagcagtccatcgccccccgccccacccgcggcccgcgggagcacctccgccgccgttctgaggcgccggaggcgggatgcggggaatgccaccacaccggtgccccccgcggcccccgggaagtccctgggcaccctcaacaatcccgccaccgtccagatccaatttgcctacgattccctgcgccgccagatcaaccgcatgctgggagacctcgcgcgggcctggtgcctggagcagaagaggcagaacatggtgctgagagaactaaccaagattaatcccaccaccgtcatgtccagcatctacggtaaggcggtggcggccaagcgcctgggggatgtcatctcagtctcccagtgcgtgcccgttaaccaggccaccgtcaccctgcgcaagagcatgagggtccctggctccgagaccatgtgctactcgcgccccctggtgtccttcagctttatcaacgacaccaagacctacgagggacagctgggcaccgacaacgagatcttcctcacaaaaaagatgacggaggtgtgccaggcgaccagccagtactacttccagtccggcaacgagatccacgtctacaacgactaccaccactttaaaaccatcgagctggacggcattgccaccctgcagaccttcatctcactaaacacctccctcatcgagaacattgactttgcctccctggagctgtactcacgggacgaacagcgtgcctccaacgtctttgacctggagggcatcttccgggagtacaacttccaggcgcaaaacatcgccggcctgcggaaggatttggacaatgcagtgtcaaacggaagaaatcaattcgtggacggcctgggggaacttatggacagtctgggtagcgtgggtcagtccatcaccaacctagtcagcacggtggggggtttgtttagcagcctggtctctggtttcatctccttcttcaaaaaccccttcggcggcatgctcattctggtcctggtggcgggggtggtgatcctggttatttccctcacgaggcgcacgcgccagatgtcgcagcagccggtgcagatgctctaccccgggatcgacgagctcgctcagcaacatgcctctggtgagggtccaggcattaatcccattagtaagacagaattacaagccatcatgttagcgctgcatgagcaaaaccaggagcaaaagagagcagctcagagggcggccggaccctcagtggccagcagagcattgcaggcagccagggaccgttttccaggcctacgcagaagacgctatcacgatccagagaccgccgccgcactgcttggggaggcagagactgagttttaa

SEQ ID NO: 95 - DNA, 2724 bp, Artificial Sequence
  DomainI SNP A733G gB5 HCMV strain Merlin
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcactctcgatccggttcagtctctcaacgcgtaacttcttcccaaacggtcagccatggtgttaacgagaccatctacaacactaccctcaagtacggagatgtggtgggggtcaataccaccaagtacccctatcgcgtgtgttctatggcccagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagccacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgaggcctgtaatctgaattgtatggtgaccatcactactgcgcgctccaaatatccttatcattttttcgccacttccacgggtgacgtggttgacatttctcctttctacaacggaaccaatcgcaatgccagctactttggagaaaacgccgacaagtttttcatttttccgaactacactatcgtctccgactttggaagaccgaattctgcgttagagacccacaggttggtggcttttcttgaacgtgcggactcggtgatctcctgggatatacaggacgaaaagaatgtcacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgcgtacgtgatgaggctataaataagttacagcagattttcaatacttcatacaatcaaacatatgaaaaatatggaaacgtgtccgtctttgaaaccactggtggtttggtagtgttctggcaaggtatcaagcaaaaatctctggtggaactcgaacgtttggccaaccgctccagtctgaatcttactcataatagaaccaaaagaagtacagatggcaacaatgcaactcatttatccaacatggaatcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaactcagcaagatcaacccgtcagccattctctcggccatttacaacaaaccgattgccgcgcgtttcatgggtgatgtcttgggcctggccagctgcgtgaccatcaaccaaaccagcgtcaaggtgctgcgtgatatgaacgtgaaggagtcgccaggacgctgctactcacgacccgtggtcatctttaatttcgccaacagctcgtacgtgcagtacggtcaactgggcgaggacaacgaaatcctgttgggcaaccaccgcactgaggaatgtcagcttcccagcctcaagatcttcatcgccgggaactcggcctacgagtacgtggactacctcttcaaacgcatgattgacctcagcagtatctccaccgtcgacagcatgatcgccctggatatcgacccgctggaaaataccgacttcagggtactggaactttactcgcagaaagagctgcgttccagcaacgtttttgacctcgaagagatcatgcgcgaattcaactcgtacaagcagcgggtaaagtacgtggaggacaaggtagtcgacccgctaccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagcgttcaccatcatcctcgtggccatagctgtagtcattatcacttatttgatctatactcgacagcggcgtttgtgcacgcagccgctgcagaacctctttccctatctggtgtccgccgacgggaccaccgtgacgtcgggcagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcagaacggtacagattctttggacggacggactggcacgcaggacaagggacagaagcccaacctactagaccgactgcgacatcgcaaaaacggctaccgacacttgaaagactctgacgaagaagagaacgtctga

SEQ ID NO: 96 - DNA, 2493 bp, Artificial Sequence
  DomainI SNP A577G gB6A HHV6A strain U1102
atgagcaagatggcagtattattcctggctgtctttttgatgaatagtgttttaatgatatattgtgatccggatcattatatcagagcgggctataatcacaaatatccttttcggatttgttcgattgccaaaggcacggatttaatgcggttcgacagagatatttcgtgctcgccgtataagtctaatgcaaagatgtcggagggttttttcatcatttacaaaacaaatatcgagacctacacttttccagtgagaacgtataaaaaagagttgacgttccaaagtagttaccgtgatgtgggtgtggtttattttctggatcggacggtgatgggtttggccatgccggtgtacgaagcaaatttagttaattctcatgcgcagtgttattccgccgtagcgatgaaacgacccgatggtacggtgtttagtgcctttcatgaggataataataaaaacaatactctaaatttatttcctctgaatttcaaatctataactaataaaagatttatcactacgaaagaaccctactttgcaaggggtcctttgtggctatattctacatcagcgtctctcaattgtattgtgacggaggctacggctaaggcgaaatacccgtttagttactttgctttgacgactggcgaaatcgtggaagggtctccgttcttcaacggttcaaacggtaaacattttgcagagccgttagaaaaattgacaatcttggaaaactatactatgatagaagatctaatgaatggtatgaatggggccactacgttagtgaggaaaatcgcttttctcgagaaagcggatactttgttttcttgggaaatcaaggaagagaatgaatcggtgtgtatgctaaagcactggaccacggtgactcacgggcttcgagcggagacgaatgagacctatcactttatctctaaggagttgacagccgctttcgtcgcccccaaggagtccttaaatcttaccgatccgaaacaaacgtgtattaagaatgaatttgaaaaaataattaatgaagtctatatgtcagattataatgatacatatagcatgaatggtagttatcaaatttttaagactacgggagatttgattttgatttggcaacctcttgtgcaaaaatctcttatgtttcttgagcagggttcggaaaaaatacgtaggaggcgagatgtgggggatgttaagtctagacatgatattctttatgtgcaattacagtatctttatgatactttgaaagattatatcaatgatgcattggggaatttggcagaatcttggtgtctcgatcaaaagcgaacgataacgatgttgcacgaacttagtaagattagtccgtcgagtatcgtgtctgaggtttacggtcgtccgatatctgcacagttgcatggtgacgtgttagctatctcgaaatgcatagaggttaatcagtcatccgttcagcttcataagagtatgcgggtcgtcgatgcaaagggagtaaggagtgaaacgatgtgttataatcggcccttggtgacgtttagttttgtgaactcgacgcctgaggttgtccctggccagctagggttagataatgaaattctgttgggtgatcataggacagaggaatgtgaaatacctagtacaaagatctttttatctggaaatcatgcgcacgtgtataccgattatacgcatacgaattcgacgcccatagaagacattgaggtattggatgcttttattagactaaaaatcgatcctcttgaaaatgccgattttaaagtactcgatttatattcgccggacgaattgagtagagcaaacgttttcgatctagagaatattcttcgtgaatataactcatataagagcgcactatatactatagaagctaaaattgctacgaatacaccttcgtatgttaatgggattaattcttttttacaagggcttggggctataggcactggattgggctcggttataagtgttacggcaggagcgcttggggatattgtgggtggagtggtgtcttttctaaaaaatccattcgggggtggtctcatgttgattttagcgatagtagttgttgtgataataattgtggttttcgttagacaaagacatgtgcttagtaagcctattgacatgatgtttccttatgccaccaatccggtgactactgtgtccagtgttacggggaccactgtcgtcaagacacctagtgttaaggatgttgacgggggcacatctgttgcggtttcggaaaaggaggagggtatggctgacgtcagtgggcaagtaagtgacgatgaatattcacaagaagatgctttaaaaatgctcaaggccataaagtccttagacgagtcctacagaagaaaaccttcgtcttctgagtctcatgcctcaaaacctagtttgatagacaggatcaggtatagaggttataagagtgtaaatgtagaagaagcgtga

SEQ ID NO: 97 - DNA, 2493 bp, Artificial Sequence
  DomainI SNP A577G gB6B HHV6B strain Z29
atgagcaagatgagagtattattcctggctgtctttttgatgaatagtgttttaatgatatattgcgattcggatgattatatcagagcgggctataatcacaaatatccttttcggatttgttcgattgccaaaggcactgatttgatgcggttcgacagagatatttcgtgttcgccatataagtctaatgcaaagatgtcggagggttttttcatcatttacaaaacaaatatcgagacctacacttttccagtgagaacatataaaaacgagctgacgttcccaaccagttaccgtgatgtgggtgtggtttattttctggatcggacggtgatgggtttggccatgccggtgtacgaagcaaatttagttaattctcgtgcgcagtgttattcagccgtagcgataaaacgacccgatggtacggtgtttagtgcctatcatgaggataataataaaaacgaaactctagaattatttcctctgaatttcaagtctgttactaataaaagatttatcactacgaaagaaccctactttgcaaggggtcctttgtggctctattctacatcggcgtctctcaattgtattgtgacggaggctacggctaaggcgaaatatccgtttagttactttgctttgacgactggtgaaatcgtggaagggtctccgttcttcgacggttcaaacggtaaacattttgcagagccgttagaaaaattgacaatcttggaaaactatactatgatagaagatctaatgaatggtatgaatggggctactacgttagtaaggaagatcgcttttctggagaaaggggatactttgttttcttgggaaatcaaggaagagaatgaatcggtgtgtatgctaaagcactggactacggtgactcacgggcttcgagcggagacggatgagacttatcactttatttctaaggagttgacagccgctttcgtcgcctccaaggagtctttaaatcttaccgatcccaaacaaacgtgtattaagaatgaatttgagaagataattacagatgtctatatgtcagattataatgatgcatacagcatgaacggtagttatcaaatttttaagactacgggagatctgattttgatttggcagcctcttgtgcaaaaatctcttatggttcttgagcagggttcagtaaacttacgtaggaggcgagatttggtggatgtcaagtctagacatgatattctttatgtgcaattacagtacctctatgatactttgaaagattatatcaacgatgccttggggaatttggcagaatcttggtgcctcgatcaaaaacgaacgataacgatgttgcacgaacttagtaagatcagtccatcgagtatcgtgtctgaggtttacggtcgtccgatatctgcacagttgcatggtgatgtgttagctatctcgaaatgcatagaagttaatcaatcatccgttcagctttataagagtatgcgggtcgtcgatgcgaagggagtaaggagtgaaacgatgtgttataatcggcccttggtgacgtttagctttgtgaactccacgcctgaggttgtccttggtcagctagggttagataatgagattctgttgggtgatcataggacagaggaatgtgagatacctagtacaaagatatttctatctggaaatcatgcacacgtgtataccgattatacgcatacgaattcgacgcccatagaagacattgaggtattggatgcttttattagactaaagatcgaccctctcgaaaatgctgattttaaactacttgatttatattcgccggacgaattgagtagagcaaacgttttcgatttagagaatattcttcgtgaatataactcatataagagcgcactatatactatagaagctaaaattgctactaatacgccgtcgtatgtcaatgggattaattcttttttacaagggcttggggctataggcactggattgggctcggttataagtgttacggcaggagcacttggggatattgtgggtggagtggtgtcttttttaaaaaatccattcgggggtggtctcatgttgattttagcgatagtagttgtcgttataataattgtggttttcgttagacaaaaacatgtgcttagtaagcctattgacatgatgtttccttatgccaccaatccggtgactactgtgtccagtgttacggggaccactgtcgtcaagacgcctagtgttaaagatgctgacgggggcacatctgttgcggtttcggaaaaagaggagggtatggctgacgtcagtggacaaataagtggtgatgaatattcacaagaagatgctttaaaaatgctcaaggccataaagtctttagacgagtcctacagaagaaaaccttcgtcttctgagtctcatgcctcaaaacctagtttgatagacaggatcaggtatagaggttataagagtgtaaatgtagaagaagcgtga

SEQ ID NO: 98 - DNA, 2469 bp, Artificial Sequence
  DomainI SNP A568G gB7 HHV7 strain RK
atgaaaattctattcctgagtgtttttataacttttagcttacagctatctctacaaacagaagctgactttgtcatgactggacacaatcagcatttaccatttcgaatttgttcaattgccaccgggacagatttagtgcgttttgacagggaggtttcttgcgcgtcttacggctctaacattaaaactaccgaaggtattttgataatttacaaaacaaagattgaagcacacacgttttctgtcagaacttttaaaaaagaacttacatttcaaacaacatatcgcgatgttggcactgtgtatttcttagatcgaactgttacaactttgccaatgccaattgaagaagtgcacatggtaaacaccgaggcgcgttgtttgtcctctatctctgtaaaacgttcggaggaagaggagtatgttgcatatcataaagatgaatatgtgaataaaacgttggatttgattccgttaaattttaaatctgatactgtcagaagatatataactactaaagaaccatttttaagaaatggtcccctctggttctattcaacatctgcatcgataaattgcatagttacagactgcattgcaaagactaaatatccttttgatttttttgctttatcaacaggggaaaccgtggaagggtcaccgttttataatggtattaattctaaaacatttaatgaaccaacggaaaaaattttgtttagaaataattatactatgctgaaaacgtttgatgatggatcaaaaggaaattttgtgacgttaactaaaatggcttttctggaaaagggaaatactattttttcatgggaagtgcagaatgaagagtcttccatttgtttgttaaagcattggatgacaatcccacacgctttacgggcagaaaatgctaacagttttcactttattgcgcaggaactaactgcttcttttgtcacaggaaaaagtaattatacgctttctgattcaaaatataattgtattaacagcaattatacttcaattttggatgagatttaccaaacgcaatacaacaattcgcatgacaaaaatggtagttatgaaatttttaaaactgagggagatttaattctgatttggcaaccgttaatacaacggaaattaacggttttggaaaatttttctaatgcttctagaaaaaggaggaagagggaattagagactaacaaagacatcgtatatgttcaactacaatacctatacgacactctgaaagattacattaacacagcactaggtaagcttgctgaagcctggtgtttagatcaaaaacgcactatcacagtgttgcacgagcttagtaaaataagtccttctggaatcatttcagcagtgtacggtaaacccatgtccgctaaattaattggtgatgtattagcagtctcaaagtgcattgaagtcaaccagacttctgttcagctacataaaagtatgagattgacaaaagattcaagttacgatgctctaagatgttattctcggccattgttaacatattcatttgcaaattcttcgaaggaaacttatttaggacaacttggtttagacaatgagatattactgggaaatcacagaacagaagaatgtgaacaatctaacacgaaaatttttttgtctggtaaattcgctcatatttttaaagactatacgtacgttaattctagtttgataacagaaatagaagctttagatgcgtttgttgacttaaacatagatcctttagaaaatgcagattttacattattggaattgtatacaaaagatgaactgagcaaagcgaacgtttttgatttggaaaccattcttagagaatacaattcttacaaaagtgcgctacaccatatagaaacaaaaattgcaactgttactccaacatatataggagggattgacacatttttcaaaggtcttggcgctcttggtcttggtttgggggctgtgctgggtgtaacggctggtgctttgggagatgtggtgaatggagttttctcttttcttaaaaatccatttggtggagcactaactatcttactaactttgggagtgattggcttagttattttcttatttttaagacataaacgattagcacaaacaccgattgatattttatttccttatacatcaaaatcaacaaattcggtacttcaagcaacgcaatcagttcaagcgcaagtgaaagaacctttagattcatctccgccttatttaaaaactaacaaagacacagaaccgcaaggtgatgacataacacacactaatgaatattcacaagtggaagctttaaaaatgttgaaagctattaaattattagatgagtcatataaaaaagcggaaatcgctgaggcaaaaaaatcacagagaccaagcctacttgaaaggattcaatacaggggctatcagaaactttcaacagaagaactgtga

SEQ ID NO: 99 - DNA, 2538 bp, Artificial Sequence
  DomainI SNP A652G gB8 HHV8 strain GK18
atgactcccaggtctagattggccaccctggggactgtcatcctgttggtctgcttttgcgcaggcgcggcgcactcgaggggtgacacctttcagacgtccagttcccccacacccccaggatcttcctctaaggcccccaccaaacctggtgaggaagcatctggtcctaagagtgtggacttttaccagttcagagtgtgtagtgcatcgatcaccggggagctttttcggttcaacctggagcagacgtgcccagacaccaaagacaagtaccaccaagaaggaattttactggtgtacaaaaaaaacatagtgcctcatatctttaaggtgcggcgctataggaaaattgccacctctgtcacggtctacaggggcttgacagagtccgccatcaccaacaagtatgaactcccgagacccgtgccactctatgagataagccacatggacagcacctatcagtgctttagttccatgaaggtaaatgtcaacggggtagaaaacacatttactgacagagacgatgttaacaccacagtattcctccaaccagtagaggggcttacggataacattcaaaggtactttagccagccggtcatctacgcggaacccggctggtttcccggcatatacagagttagggccaccgtcaattgcgagatagtggacatgatagccaggtctgctgaaccatacaattactttgtcacgtcactgggtgacacggtggaagtctcccctttttgctataacgaatcctcatgcagcacaacccccagcaacaaaaatggccttagcgtccaagtagttctcaaccacactgtggtcacgtactctgacagaggaaccagtcccactccccaaaacaggatctttgtggaaacgggagcgtacacgctttcgtgggcctccgagagcaagaccacggccgtgtgtccgctggcactgtggaaaaccttcccgcgctccatccagactacccacgaggacagcttccactttgtggccaacgagatcacggccaccttcacggctcctctaacgccagtggccaactttaccgacacgtactcttgtctgacctcggatatcaacaccacgctaaacgccagcaaggccaaactggcgagcactcacgtccctaacgggacggtccagtacttccacacaacaggcggactctatttggtctggcagcccatgtccgcgattaacctgactcacgctcagggcgacagcgggaaccccacgtcatcgccgcccccctccgcatcccccatgaccacctctgccagccgcagaaagagacggtcagccagtaccgctgctgccggcggcggggggtccacggacaacctgtcttacacgcagctgcagtttgcctacgacaaactgcgggatggcattaatcaggtgttagaagaactctccagggcatggtgtcgcgagcaggtcagggacaacctaatgtggtacgagctcagtaaaatcaaccccaccagcgttatgacagccatctacggtcgacctgtatccgccaagttcgtaggagacgccatttccgtgaccgagtgcattaacgtggaccagagctccgtaaacatccacaagagcctcagaaccaatagtaaggacgtgtgttacgcgcgccccctggtgacgtttaagtttttgaacagttccaacctattcaccggccagctgggcgcgcgcaatgagataatactgaccaacaaccaggtggaaacctgcaaagacacctgcgaacactacttcatcacccgcaacgagactctggtgtataaggactacgcgtacctgcgtactataaacaccactgacatatccaccctgaacacttttatcgccctgaatctatcctttattcaaaacatagacttcaaggccatcgagctgtacagcagtgcagagaaacgactcgcgagtagcgtgtttgacctggagacgatgttcagggagtacaactactacacacatcgtctcgcgggtttgcgcgaggatctggacaacaccatagatatgaacaaggagcgcttcgtaagggacttgtcggagatagtggcggacctgggtggcatcggaaaaacggtggtgaacgtggccagcagcgtggtcactctatgtggctcattggttaccggattcataaattttattaaacaccccctaggtggcatgctgatgatcattatcgttatagcaatcatcctgatcatttttatgctcagtcgccgcaccaataccatagcccaggcgccggtgaagatgatctaccccgacgtagatcgcagggcacctcctagcggcggagccccaacacgggaggaaatcaaaaacatcctgctgggaatgcaccagctacaacaagaggagaggcagaaggcggatgatctgaaaaaaagtacaccctcggtgtttcagcgtaccgcaaacggccttcgtcagcgtctgagaggatataaacctctgactcaatcgctagacatcagtccggaaacgggggagtga

SEQ ID NO: 100 - DNA, 675 bp, Human herpesvirus 1 strain 17
  NC_001806.29338-10012 Human herpesvirus 1 strain 17, glycoprotein gL-UL1
atggggattttgggttgggtcgggcttattgccgttggggttttgtgtgtgcgggggggcttgccttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttttggaggacttgagttaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcagccgaagcccctcaccacgccgccgcccatcatcgccacgtcggaccccaccccgcgacgggacgccgccacaaaaagcagacgccgacgaccccactcccggcgcctctaa

SEQ ID NO: 101 - DNA, 2715 bp, Human herpesvirus 2 strain HG52
  NC_001798.2c56152-53438 Human herpesvirus2 strain HG52,glycoprotein B-UL27
atgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccacgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 102 - DNA, 2517 bp, Human herpesvirus 2 strain HG52
  NC_001798.2c46570-44054 Human herpesvirus2 strain HG52,glycoprotein H-UL22
atgggccccggtctgtgggtggtgatgggggtcctggtgggcgttgccgggggccatgacacgtactggacggagcaaatcgacccgtggtttttgcacggtctggggttggcccgcacgtactggcgcgacacaaacaccgggcgtctgtggttgcccaacacccccgacgccagcgacccccagcgcggacgcttggcgcccccgggcgaactcaacctgactacggcatccgtgcccatgcttcggtggtacgccgagcgcttttgtttcgtgttggtcaccacggccgagtttcctcgggaccccgggcagctgctttacatcccaaagacctatctgctcggccggcctcggaacgcgagcctgcccgagctccccgaggcggggcccacgtcccgtccccccgccgaggtgacccagctcaagggactgtcgcacaaccccggcgcctccgcgctgttgcggtcccgggcctgggtaacattcgcggccgcgccggaccgcgaggggcttacgttcccgcggggagacgacggggcgaccgagaggcacccggacggccgacgcaacgcgccgcccccggggccgcccgcggggacaccgaggcatccgacgacgaacctgagcatcgcgcatctgcacaacgcatccgtgacctggctggccgccaggggcctgctacggactccgggtcggtacgtgtacctctccccgtcggcctcgacgtggcccgtgggcgtctggacgacgggcgggctggcgttcgggtgcgacgccgcgctcgtgcgcgcgcgatacgggaagggcttcatggggctcgtgatatcgatgcgggacagccctccggccgagatcatagtggtgcctgcggacaagaccctcgctcgggtcggaaatccgaccgacgaaaacgcccccgcggtgctccccgggcctccggccggccccaggtatcgcgtctttgtcctgggggccccgacgcccgccgacaacggctcggcgctggacgccctccggcgggtggccggctaccccgaggagagcacgaactacgcccagtatatgtcgcgggcctatgcggagtttttgggggaggacccgggctccggcacggacgcgcgtccgtccctgttctggcgcctcgcggggctgctcgcctcgtcggggtttgcgttcgtcaacgcggcccacgcccacgacgcgattcgcctctccgacctgctgggctttttggcccactcgcgcgtgctggccggcctggccgcccggggagcagcgggctgcgcggccgactcggtgttcctgaacgtgtccgtgttggacccggcggcccgcctgcggctggaggcgcgcctcgggcatctggtggccgcgatcctcgagcgagagcagagcctggcggcgcacgcgctgggctatcagctggcgttcgtgttggacagccccgcggcctatggcgcggtggccccgagcgcggcccgcctgatcgacgccctgtacgccgagtttctcggcggccgcgcgctaaccgccccgatggtccgccgagcgctgttttacgccacggccgtcctccgggcgccgttcctggcgggcgcgccctcggccgagcagcgggaacgcgcccgccggggcctcctcataaccacggccctgtgtacgtccgacgtcgccgcggcgacccacgccgatctccgggccgcgctagccaggaccgaccaccagaaaaacctcttctggctcccggaccacttttccccatgcgcagcttccctgcgcttcgatctcgccgagggcgggttcatcctggacgcgctggccatggccacccgatccgacatcccggcggacgtcatggcacaacagacccgcggcgtggcctccgttctcacgcgctgggcgcactacaacgccctgatccgcgccttcgtcccggaggccacccaccagtgtagcggcccgtcgcacaacgcggagccccggatcctcgtgcccatcacccacaacgccagctacgtcgtcacccacacccccttgccccgcgggatcggatacaagcttacgggcgttgacgtccgccgcccgctgtttatcacctatctcaccgccacctgcgaagggcacgcgcgggagattgagccgaagcggctggtgcgcaccgaaaaccggcgcgacctcggcctcgtgggggccgtgtttctgcgctacaccccggccggggaggtcatgtcggtgctgctggtggacacggatgccacccaacagcagctggcccaggggccggtggcgggcaccccgaacgtgttttccagcgacgtgccgtccgtggccctgttgttgttccccaacggaactgtgattcatctgctggcctttgacacgctgcccatcgccaccatcgcccccgggtttctggccgcgtccgcgctgggggtcgttatgattaccgcggccctggcgggcatccttagggtggtccgaacgtgcgtcccatttttgtggagacgcgaataa

SEQ ID NO: 103 - DNA, 675 bp, Human herpesvirus 2 strain HG52; identical to SEQ ID NO: 2
  NC_001798.29463-10137 Human herpesvirus 2 strain HG52, glycoprotein L-UL1

SEQ ID NO: 104 - DNA, 1065 bp, Human herpesvirus 3
  NC_001348.1114496-115560 Human herpesvirus 3 genome, glycoprotein gE
atgtttttaatccaatgtttgatatcggccgttatattttacatacaagtgaccaacgctttgatcttcaagggcgaccacgtgagcttgcaagttaacagcagtctcacgtctatccttattcccatgcaaaatgataattatacagagataaaaggacagcttgtctttattggagagcaactacctaccgggacaaactatagcggaacactggaactgttatacgcggatacggtggcgttttgtttccggtcagtacaagtaataagatacgacggatgtccccggattagaacgagcgcttttatttcgtgtaggtacaaacattcgtggcattatggtaactcaacggatcggatatcaacagagccggatgctggtgtaatgttgaaaattaccaaaccgggaataaatgatgctggtgtgtatgtacttcttgttcggttagaccatagcagatccaccgatggtttcattcttggtgtaaatgtatatacagcgggctcgcatcacaacattcacggggttatctacacttctccgtctctacagaatggatattctacaagagccctttttcaacaagctcgtttgtgtgatttacccgcgacacccaaagggtccggtacctccctgtttcaacatatgcttgatcttcgtgccggtaaatcgttagaggataacccttggttacatgaggacgttgttacgacagaaactaagtccgttgttaaggaggggatagaaaatcacgtatatccaacggatatgtccacgttacccgaaaagtcccttaatgatcctccagaaaatctacttataattattcctatagtagcgtctgtcatgatcctcaccgccatggttattgttattgtaataagcgttaagcgacgtagaattaaaaaacatccaatttatcgcccaaatacaaaaacaagaaggggcatacaaaatgcgacaccagaatccgatgtgatgttggaggccgccattgcacaactagcaacgattcgcgaagaatcccccccacattccgttgtaaacccgtttgttaaatag

SEQ ID NO: 105 - DNA, 1872 bp, Human herpesvirus 3
  NC_001348.1115808-117679 Human herpesvirus 3 genome, glycoprotein gI
atggggacagttaataaacctgtggtgggggtattgatggggttcggaattatcacgggaacgttgcgtataacgaatccggtcagagcatccgtcttgcgatacgatgattttcacaccgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtga

SEQ ID NO: 106 - DNA, 2796 bp, Human herpesvirus 3
  NC_001348.156819-59614 Human herpesvirus 3 genome, glycoprotein gB
atgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttcaaaactctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgaaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 107 - DNA, 2526 bp, Human herpesvirus 3
  NC_001348.166074-68599 Human herpesvirus 3 genome, glycoprotein gH orf37
atgtttgcgctagttttagcggtggtaattcttcctctttggaccacggctaataaatcttacgtaacaccaacccctgcgactcgctctatcggacatatgtctgctcttctacgagaatattccgaccgtaatatgtctctgaaattagaagccttttatcctactggtttcgatgaagaactcattaaatcacttcactggggaaatgatagaaaacacgttttcttggttattgttaaggttaaccctacaacacacgaaggagacgtcgggctggttatatttccaaaatacttgttatcgccataccatttcaaagcagaacatcgagcaccgtttcctgctggacgttttggatttcttagtcaccctgtgacacccgacgtgagcttctttgacagttcgtttgcgccgtatttaactacgcaacatcttgttgcgtttactacgttcccaccaaacccccttgtatggcatttggaaagagctgagaccgcagcaactgcagaaaggccgtttggggtaagtcttttacccgctcgcccaacagtccccaagaatactattctggaacataaagcgcattttgctacatgggatgcccttgcccgacatacttttttttctgccgaagcaattatcaccaactcaacgttgagaatacacgttcccctttttgggtcggtatggccaattcgatactgggccaccggttcggtgcttctcacaagcgactcgggtcgtgtggaagtaaatattggtgtaggatttatgagctcgctcatttctttatcctctggaccaccgatagaattaattgttgtaccacatacagtaaaactgaacgcggttacaagcgacaccacatggttccagctaaatccaccgggtccggatccggggccatcttatcgagtttatttacttggacgtgggttggatatgaatttttcaaagcatgctacggtcgatatatgcgcatatcccgaagagagtttggattaccgctatcatttatccatggcccacacggaggctctgcggatgacaacgaaggcggatcaacatgacataaacgaggaaagctattaccatatcgccgcaagaatagccacatcaatttttgcgttgtcggaaatgggccgtaccacagaatattttctgttagatgagatcgtagatgttcagtatcaattaaaattccttaattacattttaatgcggataggagcaggagctcatcccaacactatatccggaacctcggatctgatctttgccgatccatcgcagcttcatgacgaactttcacttctttttggtcaggtaaaacccgcaaatgtcgattattttatttcatatgatgaagcccgtgatcaactaaagaccgcatacgcgctttcccgtggtcaagaccatgtgaatgcactttctctcgccaggcgtgttataatgagcatatacaaggggctgcttgtgaagcaaaatttaaatgctacagagaggcaggctttattttttgcctcaatgattttattaaatttccgcgaaggactagaaaattcatctcgggtattagacggtcgcacaactttgcttttaatgacatccatgtgtacggcagctcacgccacgcaagcagcacttaacatacaagaaggcctggcatacttaaatccttcaaaacacatgtttacaataccaaacgtatacagtccttgtatgggttcccttcgtacagacctcacggaagagattcatgttatgaatctcctgtcggcaataccaacacgcccaggacttaacgaggtattgcatacccaactagacgaatctgaaatattcgacgcggcatttaaaaccatgatgatttttaccacatggactgccaaagatttgcatatactccacacccatgtaccagaagtatttacgtgtcaagatgcagccgcgcgtaacggagaatatgtgctcattcttccagctgtccagggacacagttatgtgattacacgaaacaaacctcaaaggggtttggtatattccctggcagatgtggatgtatataaccccatatccgttgtttatttaagcagggatacttgcgtgtctgaacatggtgtcatagagacggtcgcactgccccatccggacaatttaaaagaatgtttgtattgcggaagtgtttttcttaggtatctaaccacgggggcgattatggatataattattattgacagcaaagatacagaacgacaactagccgctatgggaaactccacaattccacccttcaatccagacatgcacggggatgactctaaggctgtgttgttgtttccaaacggaactgtggtaacgcttctaggattcgaacgacgacaagccatacgaatgtcgggacaataccttggggcctctttaggaggggcgtttctggcggtagtggggtttggtattatcggatggatgttatgtggaaattcccgccttcgagaatataataaaatacctctgacataa

SEQ ID NO: 108 - DNA, 480 bp, Human herpesvirus 3
  NC_001348.1c101649-101170 Human herpesvirus 3genome, glycoprotein gL, ORF60
atggcatcacataaatggttactgcagatagtttttttaaaaactatcacaatcgcgtattgtcttcatctccaagacgacactccgttgttttttggagccaaaccgctatcggatgtgagtttgattataacggaaccgtgcgtgtcatcggtatatgaggcgtgggactatgcggcacccccggtatcaaacctcagcgaggcgctatcgggaatcgtggttaagacaaaatgtccagtaccggaagttatactttggtttaaagacaaacaaatggcgtactggacaaatccatacgtcaccttaaaggggctggcacaatctgttggtgaagaacataaaagcggggacatacgcgatgctttgttggatgccctttccggtgtatgggtagactctactccatcttccacaaatatcccggaaaatggatgtgtctggggagccgaccgtttgttccaacgcgtatgccaatga

SEQ ID NO: 109 - RNA, 13 nt, Artificial Sequence
  Kozak consensus sequence
gccgccrccaugg

SEQ ID NO: 110 - DNA, 10 bp, Human herpesvirus 2 strain HG52
  HSV2 strain HG52 gH Kozak sequence
acgaccatgg

SEQ ID NO: 111 - protein, 31 aa, Human herpesvirus 6A
  HHV6A-mutant
YFARGPLWLYSTSASLNCIVTEATAKAKYPF

SEQ ID NO: 112 - protein, 31 aa, Human herpesvirus 6A
  HHV6A-wt
YFARGPLWLYSTSTSLNCIVTEATAKAKYPF

SEQ ID NO: 113 - protein, 31 aa, Human herpesvirus 6B; identical to SEQ ID NO: 112
  HHV6B

SEQ ID NO: 114 - protein, 31 aa, Human herpesvirus 5
  HMCV
WHSRGSTWLYRETCNLNCMVTITTARSKYPY

SEQ ID NO: 115 - protein, 32 aa, Human herpesvirus 4
  EBV1/2
YDAPGWLIWTYRTRTTVNCLITDMMAKSNSPF

SEQ ID NO: 116 - protein, 32 aa, Human herpesvirus 8
  KSHV
YAEPGWFPGIYRVRTTVNCEIVDMIARSAEPY

SEQ ID NO: 117 - protein, 31 aa, Human herpesvirus 3
  VZV
YMVAGTPGTYRTGTSVNCIIEEVEARSIFPY

SEQ ID NO: 118 - protein, 31 aa, Human herpesvirus 1
  HSV1
YNPSRVEAFHRYGTTVNCIVEEVDARSVYPY

SEQ ID NO: 119 - protein, 31 aa, Human herpesvirus 3; identical to SEQ ID NO: 118
  HSV2

SEQ ID NO: 120 - protein, 31 aa, Artificial Sequence
  HSV2mutant
YNPSRVEAFHRYGATVNCIVEEVDARSVYPY

SEQ ID NO: 121 - protein, 60 aa, Human herpesvirus 2
  HSV2 52Q-P
MGRLTSGVGTAALLVVAVGLRVVCAKYALADPSLKMADPNRFRGKNLPVLDQLTDPPGVK

SEQ ID NO: 122 - protein, 60 aa, Human herpesvirus 1
  HSV1
MGGAAARLGAVILFVVIVGLHGVRGKYALVDASLKMADPNRFRGKDLPVLDQLTDPPGVR

SEQ ID NO: 123 - protein, 23 aa, Human herpesvirus 3
  VSV
KRYFLFGHHYVYYEDYRYVREIA

SEQ ID NO: 124 - protein, 23 aa, Human herpesvirus 1
  HSV1
RRYFTFGGGYVYFEEYAYSHQLS

SEQ ID NO: 125 - protein, 23 aa, Human herpesvirus 2
  HSV2
RRYFIFGGGYVYFEEYAYSHQLS

SEQ ID NO: 126 - protein, 23 aa, Human gammaherpesvirus 4
  EBV2
QYYFQSGNEIHVYNDYHHFKTIE

SEQ ID NO: 127 - protein, 23 aa, Human herpesvirus 8
  KSHV
EHYFITRNETLVYKDYAYLRTIN

SEQ ID NO: 128 - protein, 23 aa, Human herpesvirus 5
  HCMV
LKIFIAGNSAYEYVDYLFKRMID

SEQ ID NO: 129 - protein, 25 aa, Human herpesvirus 6A
  HHV6A
TKIFLSGNHAHVYTDYTHTNSTPIE

SEQ ID NO: 130 - protein, 25 aa, Human herpesvirus 6B
TKIFLSGNHAHVYTDYTHTNSTPIE

SEQ ID NO: 131 - protein, 26 aa, Human herpesvirus 7
  HHV7
TKIFLSGKFAHIFKDYTYVNSSLITE

SEQ ID NO: 132 - DNA, 2715 bp, Artificial Sequence
  HSV1 strain 17 domain III SNP His516Pro 1547A-C
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgccaggctgcagtttacgtacaaccacatacagcgccctgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 133 - DNA, 2715 bp, Artificial Sequence
  HSV2 strain HG52 gB2 Domain III SNP encoding His513-Pro SNP 1538 A-C
atgcgcggggggggcttgatttgcgcgctggtcgtgggggcgctggtggccgcggtggcgtcggcggccccggcggccccggcggccccccgcgcctcgggcggcgtggccgcgaccgtcgcggcgaacgggggtcccgcctcccggccgccccccgtcccgagccccgcgaccaccaaggcccggaagcggaaaaccaaaaagccgcccaagcggcccgaggcgaccccgccccccgacgccaacgcgaccgtcgccgccggccacgccacgctgcgcgcgcacctgcgggaaatcaaggtcgagaacgccgatgcccagttttacgtgtgcccgcccccgacgggcgccacggtggtgcagtttgagcagccgcgccgctgcccgacgcgcccggaggggcagaactacacggagggcatcgcggtggtcttcaaggagaacatcgccccgtacaaattcaaggccaccatgtactacaaagacgtgaccgtgtcgcaggtgtggttcggccaccgctactcccagtttatggggatattcgaggaccgcgcccccgttcccttcgaggaggtgatcgacaagattaacgccaagggggtctgccgctccacggccaagtacgtgcggaacaacatggagaccaccgcgtttcaccgggacgaccacgagaccgacatggagctcaagccggcgaaggtcgccacgcgcacgagccgggggtggcacaccaccgacctcaagtacaacccctcgcgggtggaggcgttccatcggtacggcacgacggtcaactgcatcgtcgaggaggtggacgcgcggtcggtgtacccgtacgatgagtttgtgctggcgacgggcgactttgtgtacatgtccccgttttacggctaccgggaggggtcgcacaccgagcacaccagctacgccgccgaccgcttcaagcaggtcgacggcttctacgcgcgcgacctcaccacgaaggcccgggccacgtcgccgacgacccgcaacttgctgacgacccccaagtttaccgtggcctgggactgggtgccgaagcgaccggcggtctgcaccatgaccaagtggcaggaggtggacgagatgctccgcgccgagtacggcggctccttccgcttctcctccgacgccatctcgaccaccttcaccaccaacctgaccgagtactcgctctcgcgcgtcgacctgggcgactgcatcggccgggatgcccgcgaggccatcgaccgcatgtttgcgcgcaagtacaacgccacgcacatcaaggtgggccagccgcagtactacctggccacggggggcttcctcatcgcgtaccagcccctcctcagcaacacgctcgccgagctgtacgtgcgggagtacatgcgggagcaggaccgcaagccccggaatgccacgcccgcgccactgcgggaggcgcccagcgccaacgcgtccgtggagcgcatcaagaccacctcctcgatcgagttcgcccggctgcagtttacgtataaccacatacagcgccccgtgaacgacatgctggggcgcatcgccgtcgcgtggtgcgagctgcagaaccacgagctgactctctggaacgaggcccgcaagctcaaccccaacgccatcgcctccgccaccgtcggccggcgggtgagcgcgcgcatgctcggagacgtcatggccgtctccacgtgcgtgcccgtcgccccggacaacgtgatcgtgcagaactcgatgcgcgtcagctcgcggccggggacgtgctacagccgccccctggtcagctttcggtacgaagaccagggcccgctgatcgaggggcagctgggcgagaacaacgagctgcgcctcacccgcgacgcgctcgagccgtgcaccgtgggccaccggcgctacttcatcttcggcgggggctacgtgtacttcgaggagtacgcgtactctcaccagctgagtcgcgccgacgtcaccaccgtcagcaccttcatcgacctgaacatcaccatgctggaggaccacgagtttgtgcccctggaggtctacacgcgccacgagatcaaggacagcggcctgctggactacacggaggtccagcgccgcaaccagctgcacgacctgcgctttgccgacatcgacacggtcatccgcgccgacgccaacgccgccatgttcgcggggctgtgcgcgttcttcgaggggatgggggacttggggcgcgcggtcggcaaggtagtcatgggagtagtggggggcgtggtgtcggccgtctcgggcgtgtcctcctttatgtccaaccccttcggggcgcttgccgtggggctgctggtcctggccggcctggtcgcggccttcttcgccttccgctacgtcctgcaactgcaacgcaatcccatgaaggccctgtatccgctcaccaccaaggaactcaagacttccgaccccgggggcgtgggcggggagggggaggaaggcgcggaggggggcgggtttgacgaggccaagttggccgaggcccgagaaatgatccgatatatggctttggtgtcggccatggagcgcacggaacacaaggccagaaagaagggcacgagcgccctgctcagctccaaggtcaccaacatggttctgcgcaagcgcaacaaagccaggtactctccgctccacaacgaggacgaggccggagacgaagacgagctctaa

SEQ ID NO: 134 - DNA, 2796 bp, Artificial Sequence
  VZV gB3 strain Dumas SNP encoding His526Pro 1580A-C
atgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccaactcggacatgtccggattatcaccttggtaaaaactttacagagggtattgctgttgtttataaagaaaacattgcagcgtacaagtttaaggcgacggtatattacaaagatgttatcgttagcacggcgtgggccggaagttcttatacgcaaattactaatagatatgcggatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcctgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcattatacttcaaaactctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgaaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccactcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaa

SEQ ID NO: 135 - DNA, 207 bp, Human herpesvirus 6A
  Coding region of iciU83A-N cDNA of human chromosomally integrated iciHHV-6A with Kozak site
gtcgaaatgtccattcggctttttattggtttcttttatacggcatatattggtatggctatcggatttatatgtagttcccccgatgcggagctgttttccgaaaaatcacgtatttcgtcttctgtcttgttaggatgtttgttgtgttgcatggattggtccgctgccgtacccgtctggtttggagcagggctcgatgtgtga

SEQ ID NO: 136 - protein, 66 aa, Human herpesvirus 6A
  iciU83A-N polypeptide encoded by human chromosomally integrated iciHHV-6A
MSIRLFIGFFYTAYIGMAIGFICSSPDAELFSEKSRISSSVLLGCLLCCMDWSAAVPVWFGAGLDV